(19) United States
(12) Patent Application Publication
(10) Pub. No.: US 2026/0035648 A1
(43) Pub. Date: Feb. 5, 2026

(54) CELL CULTURE CONTAINERS AND FLUID HANDLING FOR SCALABLE CELL MANUFACTURING

(71) Applicant: Cellino Biotech, Inc., Cambridge, MA (US)

(72) Inventors: Matthias Wagner, Cambridge, MA (US); Ed Tekeian, Cambridge, MA (US); Lydia Skrabonja, Cambridge, MA (US); Christopher Harrison, Cambridge, MA (US); Kien Tran, Cambridge, MA (US); Catherine Pilsmaker, Arlington, MA (US); Ozge Whiting, Cambridge, MA (US); Blair Morad, Cambridge, MA (US); Shuhang Wang, Cambridge, MA (US); Matthew Sullivan, Westwood, MA (US); Scott Luro, Somerville, MA (US); Maya Berlin-Udi, Acton, MA (US); Jose Valdez, Cambridge, MA (US); Abhijit Kulkarni, Cambridge, MA (US); Alexander Stange, Somerville, MA (US); Joshua Blouwolff, Cambridge, MA (US); Jean Carlos Serrano, Cambridge, MA (US); Daeyoung Kim, Cambridge, MA (US); Erik Snow, Cambridge, MA (US); Ethan Goldman, Cambridge, MA (US)

(21) Appl. No.: 19/285,250

(22) Filed: Jul. 30, 2025

Related U.S. Application Data
(60) Provisional application No. 63/677,268, filed on Jul. 30, 2024.
(60) Provisional application No. 63/677,269, filed on Jul. 30, 2024.
(60) Provisional application No. 63/677,281, filed on Jul. 30, 2024.
(60) Provisional application No. 63/689,112, filed on Aug. 30, 2024.
(60) Provisional application No. 63/735,119, filed on Dec. 17, 2024.

(51) Int. Cl. C12M 1/00; C12M 1/36

(52) U.S. Cl. CPC C12M 29/04; C12M 23/22; C12M 29/12; C12M 41/48

(57) ABSTRACT

Systems and methods for cell culturing are disclosed herein. A fluidic exchange system for cell culturing comprises a cell culture cassette, comprising: a cell culture chamber having a cell culture and fluid media; and one or more pluggable ports fluidically connected to the cell culture chamber; a fluid media cassette, comprising: one or more liquid storage compartments; and one or more pluggable ports fluidically connected to the one or more liquid storage compartments; and a cassette coupler configured to fluidically couple the cell culture cassette and the fluid media cassette.

### RELATED APPLICATIONS

This application claims the benefit of priority to U.S. Provisional Application No. 63/677,268, filed Jul. 30, 2024, U.S. Provisional Application No. 63/677,269, filed Jul. 30, 2024, U.S. Provisional Application No. 63/677,281, filed Jul. 30, 2024, U.S. Provisional Application No. 63/689,112, filed Aug. 30, 2024, U.S. Provisional Application No. 63/735,119, filed Dec. 17, 2024, each of which is incorporated herein by reference in its entirety.

### BACKGROUND

Autologous induced pluripotent stem cell (iPSC)-derived regenerative cell therapies that utilize the patient's own cells offer a promising avenue for addressing a variety of medical conditions. They minimize immune-related complications, and are exceptionally well-suited to meet the needs of an aging and increasingly diverse patient population.

Despite significant medical advantages to patients, the current artisanal cell manufacturing methods of generating autologous iPSC-derived therapies face substantial challenges in terms of scalability. These issues pertain to cost-effectiveness and volume production, rendering autologous cell therapies economically unfeasible. Currently, iPSCs are manufactured in high-grade clean rooms by scientists over 10-12 weeks. Somatic cells, like skin fibroblasts or blood cells, are isolated and then cultured in dishes. Scientists introduce reprogramming factors, such as Oct4, Sox2, Klf4, and c-Myc, to these cells, often using Sendai viral vectors. Once pluripotent colonies form, researchers carefully pick and transfer these colonies under a microscope to new culture dishes for an extensive stabilization and vector clearing phase, followed by expansion and harvest. Resulting iPSCs are subjected to quality control (QC) release testing and cryopreserved for long-term storage.

This manual process is time-consuming and expensive, and in its current form cell therapies would only be available to those who are able to pay the significant cost involved. In addition, many cell therapy providers would have a difficult time even getting regulatory approval because of scaling issues in the late clinical trial stages. In addition, manual processes may be subject to many delays due to quality control issues, staffing issues, and other problems. Thus, there is a need in the art for scalable, automated or semi-automated solutions to enable wide-scale and efficient adoption of regenerative cell therapies, and these solutions must address the myriad technical challenges involved in automating a multi-month process performed by skilled technicians.

### BRIEF SUMMARY

In an exemplary embodiment, a fluidic exchange system for cell culturing, comprising a cell culture cassette, comprising: a cell culture chamber having a cell culture and fluid media; and one or more pluggable ports fluidically connected to the cell culture chamber; a fluid media cassette, comprising: one or more liquid storage compartments; and one or more pluggable ports fluidically connected to the one or more liquid storage compartments; and a cassette coupler configured to fluidically couple the cell culture cassette and the fluid media cassette.

In another exemplary embodiment, the cassette coupler aseptically connects with the one or more pluggable ports of the cell culture cassette and aseptically connects with the one or more pluggable ports of the fluid media cassette.

In a further exemplary embodiment, a first liquid storage compartment of the fluid media cassette stores fresh fluid media to replenish the fluid media in the cell culture chamber of the cell culture cassette.

In yet another exemplary embodiment, a second liquid storage compartment of the fluid media cassette receives used fluid media from the cell culture cassette.

In an exemplary embodiment, the fluidic exchange system further comprises a liquid transfer handler to initiate fluid flows between the cell culture cassette and the fluid media cassette.

In another exemplary embodiment, the liquid transfer handler circulates media in at least one of continuous flow mode and stopped-flow mode.

In a further exemplary embodiment, the fluid media cassette further comprises at least one bypass valve between a first liquid storage compartment and a second liquid storage compartment to enable fluid mixing operations.

In yet another exemplary embodiment, the fluid media cassette further comprises at least one pinch valve between a first liquid storage compartment and a first pluggable port.

In an exemplary embodiment, the fluid media cassette further comprises a semiporous membrane configured to enable filtration flows between a first liquid storage compartment and a second liquid storage compartment.

In another exemplary embodiment, the cell culture is adhered to a semi-transparent surface of the cell culture chamber.

In a further exemplary embodiment, the semi-transparent surface comprises an optical film for optical imaging of the cell culture and optical cell culture management.

In yet another exemplary embodiment, the fluidic exchange system further comprises a robotic system configured to move at least one of the cell culture cassette and the fluid media cassette to the cassette coupler.

In an exemplary embodiment, the cell culture cassette provides a closed, sterile cell culture environment.

In another exemplary embodiment, the fluid exchange system simultaneously replenishes the fluid media in the cell culture cassette with fresh fluid media in the fluid media cassette and removes waste media from the cell culture cassette to the fluid media cassette.

In a further exemplary embodiment, the one or more pluggable ports of the cell culture cassette and the one or more pluggable ports of the fluid media cassette are self-sealing.

In yet another exemplary embodiment, the cell culture cassette further comprises a gas-permeable membrane allowing gas exchange while maintaining sterility of the cell culture chamber.

In an exemplary embodiment, the cassette coupler comprises a sterilant port to introduce a sterilant into the sealed interior space.

In another exemplary embodiment, the liquid transfer handler comprises a peristaltic pump controlling fluid flow between the cell culture cassette and the fluid supply cartridge.

In a further exemplary embodiment, the cell culture chamber comprises a temperature control material for maintaining the cell culture cassette at a predetermined temperature during fluid exchange operations.

In yet another exemplary embodiment, the cell culture cassette comprises optical fiducial markers for automated alignment and imaging of the cell culture chamber.

### BRIEF DESCRIPTION OF THE DRAWINGS

FIG. 1 is a block diagram illustrating an autonomous cell manufacturing platform in accordance with various implementations.

FIGS. 2A-B are diagrams illustrating an example cell culture cassette for use in a cell culture system in accordance with various implementations.

FIGS. 3A-B are diagrams illustrating another example cell culture cassette for use in a cell culture system in accordance with various implementations.

FIGS. 4A-B are diagrams illustrating an example cell culture cassette and fluidic cartridge for use in a cell culture system in accordance with various implementations.

FIG. 5 is a diagram illustrating a pluggable fluidic cartridge in accordance with various implementations.

FIG. 6 is a diagram illustrating steps for manufacturing a fluidic cassette in accordance with various implementations.

FIGS. 7A-C are diagrams illustrating a cassette for use in a cell culture system in accordance with various implementations.

FIG. 8 is a diagram illustrating a fluidic cassette for use in a cell culture system in accordance with various implementations.

FIG. 9 is a diagram illustrating a closed cell culture cassette for use in a cell culture system in accordance with various implementations.

FIGS. 10A-D are diagrams illustrating a method of fluid washing in a closed fluidic cassette in accordance with various implementations.

FIGS. 11A-C are diagrams illustrating another method of fluid washing in a closed fluidic cassette in accordance with various implementations.

FIGS. 12A-C are diagrams illustrating a method for liquid replacement in a closed cassette in accordance with various implementations.

FIG. 13 is a diagram illustrating a cell culture chamber in accordance with various implementations.

FIG. 14 is a diagram illustrating a fluidic cell culture chamber in accordance with various implementations.

FIG. 15 is a diagram illustrating a closed cell culture container and a pump system in accordance with various implementations.

FIG. 16 illustrates a method of applying adhesion reduction to a cell culture in accordance with various implementations.

FIG. 17 is a diagram illustrating a fluidic cell culture cassette in accordance with various implementations.

FIGS. 18A-D are diagrams illustrating various cell culture containers and frames in accordance with various implementations.

FIG. 19 is a diagram illustrating applying rotational motion on a closed cell culture container in accordance with various implementations.

FIG. 20 is a diagram illustrating a modular bioprocessing system that uses a plurality of cassettes in accordance with various implementations.

FIG. 21 is a diagram illustrating a cell culture container in accordance with various implementations.

FIGS. 22A-C are diagrams illustrating a robot-mounted fluid management system for a cell culture system in accordance with various implementations.

FIG. 23 is a diagram illustrating a cell culture system in accordance with various implementations.

FIG. 24 is a diagram illustrating a modular bioprocessing system in accordance with various implementations.

FIGS. 25A-D are diagrams illustrating a reusable magnetically-actuated aseptic connector in accordance with various implementations.

FIGS. 26A-D are diagrams illustrating an example multi-channel aseptic connector in accordance with various implementations.

FIGS. 27A-C are diagrams illustrating reusable aseptic connectors that are compatible with high-temperature sterilization in accordance with various implementations.

FIG. 28 is a diagram illustrating a fluidic system for a cell culture system in accordance with various implementations.

FIG. 29 illustrates a diagram of an aseptic connector in accordance with various implementations.

FIGS. 30A-D are diagrams illustrating a method of inserting connectors into a sealed block in accordance with various implementations.

FIGS. 31A-D are diagrams illustrating operation of a reusable aseptic connection system in accordance with various implementations.

FIGS. 32A-B are diagrams illustrating an aseptic connector system in accordance with various implementations.

FIG. 33 is a diagram illustrating a fluidic management system for use in a cell culture system in accordance with various implementations.

FIGS. 34A-B are diagrams illustrating an aseptic coupler for use in a cell culture system in accordance with various implementations.

FIGS. 35A-H are diagrams illustrating a method for establishing an aseptic connection using an aseptic connector in accordance with various implementations.

FIG. 36 is a diagram illustrating a fluidic connection system in accordance with various implementations.

FIG. 37 is a diagram illustrating a bubble removal system in accordance with various implementations.

FIGS. 38A-B are diagrams illustrating a system and method for removing bubbles in fluid connectors in accordance with various implementations.

FIG. 39A is an image of an automation-compatible closed fluidic cell growth cassette system with normally-closed pluggable ports in accordance with various implementations.

FIG. 39B is a diagram of a normally-closed port used in the cassette shown in FIG. 1A in accordance with various implementations.

FIG. 39C is an image of an example implementation of automation-compatible pipette tips that are compatible with the cassette shown in FIG. 1A in accordance with various implementations.

FIG. 40 is a diagram of an operating environment enabled by pluggable fluidic cassettes in accordance with various implementations.

FIG. 41 is a diagram illustrating another operating environment for performing batched bioprocesses in accordance with various implementations.

FIG. 42 is a diagram illustrating another operating environment for pluggable cassette-based systems in accordance with various implementations.

FIG. 43 is a diagram illustrating another operating environment utilizing shared equipment by multiple cell batches in accordance with various implementations.

FIG. 44 is a diagram illustrating another operating environment supporting aseptic normally-closed pluggable cassette-based systems in accordance with various implementations.

FIGS. 45A-F are diagrams illustrating utilization of a multi-part fluidic cell culture chamber in accordance with various implementations.

FIG. 46 is a diagram of another multi-part fluidic cell culture chamber in accordance with various implementations.

These and other features of the present implementations will be understood better by reading the following detailed description, taken together with the figures herein described. The accompanying drawings are not intended to be drawn to scale. For purposes of clarity, not every component may be labeled in every drawing.

### DETAILED DESCRIPTION

The systems and methods disclosed herein include an autonomous cell manufacturing platform for efficient and scalable production of cells (e.g., iPSCs, differentiated cells) for use in cell therapies. The cell manufacturing platform utilizes optical bioprocesses such as optical imaging and optical-based cell culture management to continuously monitor and control cell culture processes without the need for constant human intervention. Cells are cultured, monitored, and managed in compact closed systems, such as closed cassettes, to provide a mobile, sterile cell culture environment while allowing for high multi-patient throughput in the overall system. The manufacturing platform also includes incubation spaces for cells to expand or grow over long periods of time, a fluid management system to inject and remove fluid media from the closed cassettes, and robotic elements to move the closed cassettes around the platform. The manufacturing platform utilizes artificial intelligence (AI) to analyze cell culture images and make determinations about cell interventions (e.g., cell removal, cell harvesting, media changes).

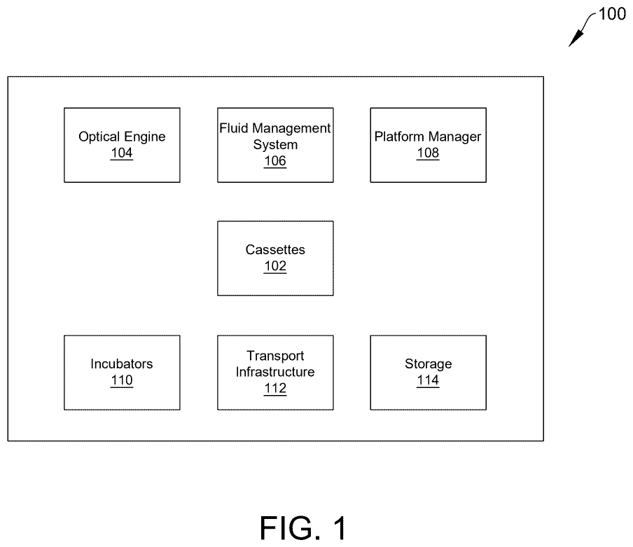

FIG. 1 is a block diagram illustrating an autonomous cell manufacturing platform 100 in accordance with various implementations. The platform 100 enables scalable, autonomous, and efficient production of cells for cell therapies. The platform 100 may provide a controlled, sterile environment for the manufacturing of cells for cell therapies. For example, the platform 100 may be a sealed enclosure with minimal interfaces to external environments and may be sterilized between uses. The platform 100 may be configured to adhere to various regulatory requirements for the production of cell and drug products administrable to patients.

Platform 100 includes a plurality of cassettes 102. Each cassette 102 may support a cell culture. The cell culture may start as source cells that undergo a cell culture process to produce an output cell product. For example, the source cells may be somatic cells that undergo reprogramming and expansion into output iPSC cells. In another example, the source cells may be iPSC cells that undergo differentiation and expansion into differentiated cells for use in cell therapies. Example cell culture processes that may be performed by the platform 100 may include, but are not limited to, cell expansion, cell reprogramming, cell differentiation, cell rejuvenation, cell regeneration, cell gene editing, cell transdifferentiation, cell purification, and cell clonalization, Each cassette 102 may be a closed, sterile system that prevents contamination of the cell samples and allows for multi-sample and/or multi-patient processing using shared infrastructure.

Each cassette 102 may include one or more cell culture chambers, which may be closed fluidic chambers for growing cells (e.g., adherent cells). The cell culture chambers may include at least one transparent surface that includes an optical film upon which the cells adhere. The optical film may be flat and permanently attached to the first surface of the cell culture chamber. The optical film may be a multi-layered composition that includes various layers to enable selective light absorption, promote cell adherence, and prevent leaching of materials into the cell culture chamber. The optical film may enable optical-based label-free imaging and optical-based cell manipulation and removal techniques (e.g., using a laser). For example, the optical film may be configured to transmit wavelengths within certain wavelength ranges for imaging applications and at least partially absorb wavelengths in other wavelength ranges for optical removal applications. The cassettes 102 may be in a format that allows for observation of the cell culture at regular intervals. The cassettes 102 may also include various other components to enable autonomous optical processes, transport, and fluid exchange. For example, the cassettes 102 may include fiducials used to align imaging equipment, ports and tubing to enable fluid exchanges, and handles or other mechanical features to enable gripping, rotation, transport, or other physical manipulations of the cassette 102. In some implementations, the cassette 102 may have different designs and configurations for different purposes (e.g., expansion cassette, growth/maintenance cassette, differentiation cassette, harvesting cassette).

The platform 100 also includes an optical engine 104, which is configured to provide optical imaging and cell intervention functionality on the platform 100. Optical-based processes allow cell cultures to be monitored and managed without mechanical means, and thus not breaking the closed, sterile environment of the cassettes 102. The optical engine 104 may be located in a particular location within the platform 100, and cassettes 102 may be moved to the optical engine 104 from a storage location by robotic means for performing imaging and cell management functions. The optical engine 104 may be configured to provide label-free imaging suitable for long-term cell culture observation, although some implementations may include fluorescent imaging capability for immunofluorescent or other labeled images. The optical engine 104 may be configured to collect time-series images of cell cultures in the cassettes, which may be used by machine learning models to analyze cell growth, make predictions on future cell growth, and make determinations about interventions to perform on the cell culture.

The optical engine 104 may also be configured to function as a cell removal tool, or to perform other methods of optical-based manipulation of the cell culture (e.g., cell poration, removal of ECM) in the cassettes 102. The optical engine 104 may be configured to target and remove cells at a regional, cluster-specific, and/or cell-specific level. Removal, in this context, may include selective destruction and/or removal of cells or cell regions, and non-destructive operations on cells (including intracellular delivery of compounds into cells or extraction of compounds from cells). The optical engine 104 may also be used to perform cell operations on a cell culture, such as splitting cell colonies into multiple sub-colonies, translating cell colonies across a cell culture surface, reducing confluence, surface area, or density of cell colonies by selective removal, and clonalization of cell colonies by repeated culling of portions of the cell colonies. In some implementations, the platform 100 may include more than one optical engine 104 that is shared by the cassettes 102.

One example implementation of the optical engine 104 is a laser-based system. The optical engine 104 may emit light within a first wavelength range for imaging cells in the cassettes 102. The optical engine 104 may also emit laser pulses within a second wavelength range that are designed to remove cells from the cell growth surface. Removal may be effectuated by, for example, heat transfer of energy from the laser pulses to the cells to dislodge/kill them, or conversion of optical energy into mechanical energy via the formation of microbubbles that kill and/or dislodge the cells.

The platform 100 also includes a fluid management system 106 that is configured to handle the injection and removal of fluids from the cassettes 102. Fluid media include nutrients necessary for cells to grow, and cells expel waste into the fluid media. Thus, the fluid media must be periodically refreshed for cells to grow and be maintained in a healthy state. The fluid management system 106 may be located in a particular location within the platform 100, and cassettes 102 may be moved to the fluid management system 106 from a storage location by robotic means for performing fluidic exchange functions. The fluid management system 106 may include a receptacle for holding cassettes 102 in place during the fluid exchange. In some implementations, the receptacle may be configured to rotate, translate, or shake/vibrate the cassettes to achieve various fluid manipulation functions. The fluid management system 106 may also include tubing, pipetting, ports, and connectors to connect the cassettes with fluid and waste reservoirs. In some implementations, the fluid management system 106 is configured to aseptically connect to the cassettes to prevent contamination of the cell culture during fluid exchanges. In some implementations, the platform 100 may include more than one fluid management system 106 that is shared by the cassettes 102.

The platform 100 also includes a platform manager 108 configured to monitor and control the other components of the platform 100. The platform manager 108 may be, for example, a combination of on-premises and cloud computing resources that provide data collection, data analysis, and control functions. The platform manager 108 may be configured to gather data from a range of sources, organize the data in a manner that allows it to make predictions of success/quality/functionality of the cell culture, and in many cases do so on a cell-by-cell, cluster-by-cluster, or region-by-region basis. The platform manager 108 may utilize various artificial intelligence and machine learning models to monitor, analyze, and manage cell culture processes. The platform manager 108 may control the optical engine 104 according to cell management algorithms (for example, to maintain a certain cell density, to maintain certain exclusion areas within the cell culture container), in a timed manner (for example, delivering gene-activating or gene-editing compounds to cells at a specific interval), and/or as a result of predictions made by the platform manager 108 (for example, removal of cells predicted not to yield the desired phenotype or optimal level of function).

The platform 100 may also include one or more incubators 110. The incubators 110 may serve as storage locations for the cassettes 102 during cell growth, expansion, or maintenance, when the cassettes 102 are not being transported to the optical engine 104 or the fluid management system 106. The incubators 110 may be maintained at certain temperatures conducive for cell growth. The platform 100 may also include transport infrastructure 112 for moving cassettes 102 within the platform (e.g., from the incubators 110 to the optical engine 104 and back). The transport infrastructure 112 may include, for example, robotic arms that can grasp and move the cassettes, and/or rails that can transport the cassettes 102 from one location to another. The platform 100 may also include storage space 114, which may be used to store consumables within the platform. Such consumables may include, for example, fluids, pipettes, connectors, and other one-time use components. Such storage spaces may be temperature regulated, for example at 4° C. for the purpose of storing reagents or media. The platform 100 may include other components not illustrated in FIG. 1, such as interior/exterior interfaces for manual intervention (e.g., sterile glove ports) or for moving objects in and out of the platform 100 (e.g., load locks). The platform 100 may be built in an isolator model, in which the interior is aseptically separated from the external environment, or in the form of a biosafety cabinet, in which the interior is accessible but protected via airflows designed to prevent external contamination from entering. In some implementations, the platform 100 may include more than one optical engine 104 and/or fluid management system 106.

### Centrifugation-Compatible Fluidic Cassette

The exchange of fluids (e.g., cell media) while retaining cells in suspension, as well as adjusting the concentration of cell products for subsequent re-suspension, seeding, cryopreservation, etc., are typically done using centrifuges in tubes or similar consumables. In closed aseptic cell processing systems, centrifugation may be integrated into the aseptic tubing set or cartridge. However, the resulting setup is significantly complex and bulky, suited mostly to larger-volume suspension cell processing. Additionally, these subsystems may not be compatible with all-liquid designs and compact fluidic cell culture cassettes. Thus, there is a need in the art for methods to integrate centrifugation abilities into smaller profile cell culture containers.

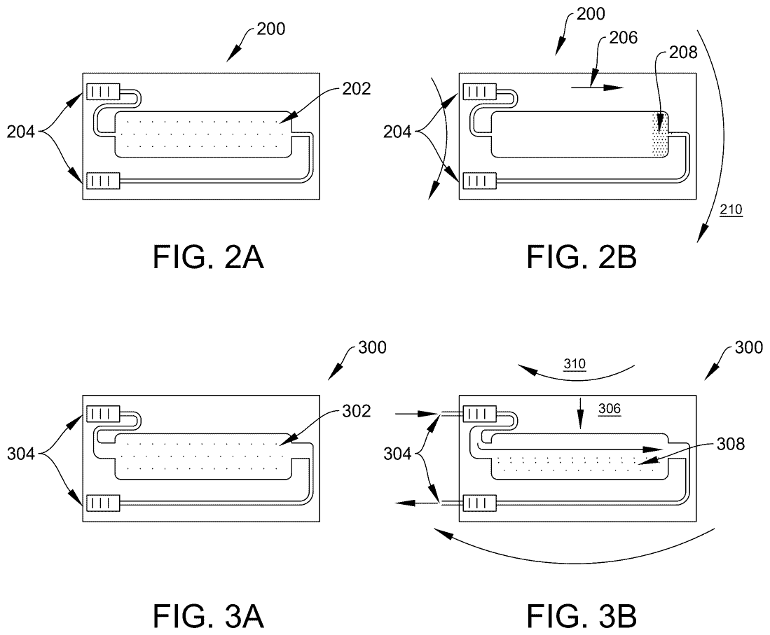

Systems and methods disclosed herein include designs for pluggable fluidic cell culture cassettes that are mechanically and fluidically compatible with centrifugation for cell concentration and low suspended cell loss media exchange. FIGS. 2A-B are diagrams illustrating an example cell culture cassette 200 for use in a cell culture system in accordance with various implementations. FIG. 2A illustrates the cassette 200 and a method for adjusting cell concentration (for example, for harvesting in a concentrated volume, re-seeding on a growth surface in a confined area, or re-suspension in a new media). The cassette 200 includes a fluidic cell culture chamber 202, shown in FIG. 2A with suspended cells distributed throughout, and pluggable ports 204 used for media and cell operations. The pluggable ports 204 may remain sealed even under pressure or vacuum conditions, unless they are opened. FIG. 2B shows the cassette 200 with an acceleration 206 applied which pushes relatively dense cells 208 to one end of the cell culture chamber 202. In some implementations, the acceleration 206 may be provided by gravity alone. In other implementations, the acceleration 206 may be applied using a rotation motion 210, for example by use of a rotating element that the cassette 200 attaches to, or rapid motion of a robotic arm. Once the cells are concentrated at one end of the chamber, as indicated by cells 208, close to the outlet attached to one of the pluggable ports, they may be harvested in concentrated form, allowed to seed a surface in this configuration, or re-suspended in new media. In some implementations, fluid flow from the “destination” port may flow at the same time as the acceleration 206 is applied, to exchange fluid without cell loss, or to suspend the concentrated cells in a new liquid.

FIGS. 3A-B are diagrams illustrating another example cell culture cassette 300 for use in a cell culture system in accordance with various implementations. In this example, cells may be sequestered using acceleration while media is exchanged. The cassette 300 includes a fluidic cell culture chamber 302, shown in FIG. 3A with suspended cells distributed throughout, and pluggable ports 304 used for media and cell operations. FIG. 3B shows an example centrifuge operation, in which acceleration 306 is applied to shift cells to a region 308. The acceleration 306 may be performed using gravity alone, or applied via rotational motion 310. During the acceleration 306, or after concentrating cells, the pluggable ports 304 may be used to exchange liquid in the cassette. Because of the localization of cells, the fluid may be exchanged with minimal loss of suspended cells. Various combinations of acceleration (including centrifugation) and flows are possible in the systems and methods contemplated herein. Cassettes specifically designed to combine gravity- or centrifugation-based forces on cells combined with fluid flows, as an operational step (rather than combined with growth chambers), are also contemplated herein.

### Supply Cartridge System for Fluidic Cassette

Cell culture systems with longer duration cell culture processes can greatly benefit from reconnectable aseptic connections. Transfers from fresh media/reagents or to waste/cell product may be accomplished using intermediate consumables (pipette tips or other temporary reservoirs) to isolate cassettes from liquid sources/sinks. However, this may create additional complexity. Even more complex is the case in all-fluidic cassettes in which liquid must be added and removed simultaneously to maintain a steady pressure. Finally, multi-patient environments are particularly complex to address because of the need to strictly prevent cross-contamination. Thus, there is a need in the art to keep patient-specific cell culture containers isolated and aseptic in a multi-patient cell culture system environment.

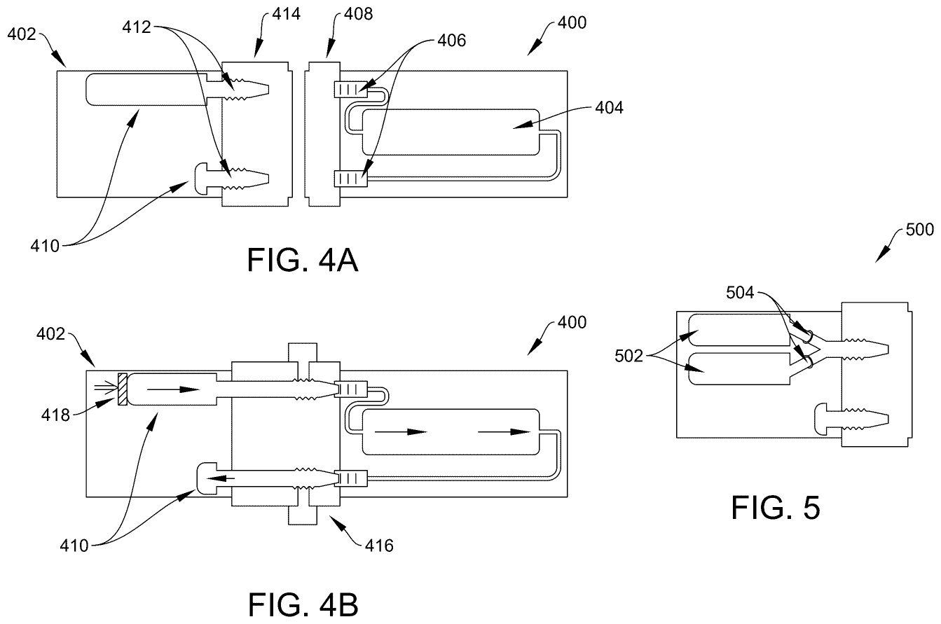

The systems and methods disclosed herein include a cassette-dedicated or patient-dedicated supply cartridge, which may be used for a single fluidic operation or several fluidic operations, and support bidirectional flow out of and into the cartridge. FIGS. 4A-B are diagrams illustrating an example cell culture cassette 400 and a fluidic cartridge 402 for use in a cell culture system in accordance with various implementations. The cassette 400 includes at least one cell culture chamber 404, with pluggable fluidic connectors 406 housed in an aseptic connector 408. The fluidic cartridge 402 includes expandable/contractible fluidic reservoirs 410 (one full, for example with fresh cell media, and one empty) attached to plug connectors 412 housed within an outside aseptic connector 414.

FIG. 4B shows the fluidic cartridge 402 plugged into the cassette 400 via a mating mechanism 416 that sterilizes external doors, removes them, and allows the pluggable fluidic connectors 406 of the cassette 400 to connect with the plug connectors 412 of the fluidic cartridge 402. Subsequently, a pressure mechanism 418 (which may be a roller, for example) squeezes one of the reservoirs 410, causing media in the reservoir to be transported into the cassette 400, replacing the media in the cell culture chamber, with waste pushed into the other reservoir 410. Reservoirs in the fluidic cartridge 402 may be bags, containers with air pressure/vacuum applied or peristaltic pumping, syringe pumps or containers, or combinations thereof.

FIG. 5 is a diagram illustrating a pluggable fluidic cartridge 500 in accordance with various implementations. In cartridge 500, a single pluggable connector may be connected to two reservoirs 502. Such a configuration may be used to generate mixed fluids with varying concentrations, to deliver different fluids at different timepoints, or to collect fluids in two reservoirs at different timepoints (for example, collecting waste media in one, and a cell product or cell sample in another). In some implementations, multiple reservoirs may be passively coupled, or have pinch valve points 504 to allow precise timing/routing of liquids.

### Pre-Filled Fluidic Cassettes for Bioprocessing

Fluidic cassettes for cell culture and cell processing are currently manufactured, shipped, and stored post-sterilization with air in the cell culture chambers, tubing, and other cell processing components. This means conducting initial liquid fills and coating of (sometimes specific) interior portions of the cassette are done as part of the cell manufacturing process. However, this results in a significant disadvantage of scale (i.e., repeated filling and pre-coating of a single cassette), high variability (user and unit-to-unit differences) in coating efficiency and uniformity, bubble entrapment, and additional opportunities for contamination. Thus, there is a need in the art for simpler pre-processing of cassettes before use in a cell culture system.

Systems and methods disclosed herein include sterile fluidic cassettes that are manufactured, shipped, and stored pre-filled with liquid that is exchanged at the start of bioprocessing. The cassettes may be cell growth cassettes, or function-specific cassettes (centrifugation, filtration, sorting, intracellular delivery, etc.), or fluidic supply cartridges that supply cassettes. The present implementations may enable a coordinated manufacturing workflow. For example, component surfaces may be pre-treated (e.g., with plasma or other treatment), or coated with processes that use temporary masks, including but not limited to photoresist (e.g., non-stick/anti-biofouling coatings, or coatings that promote adhesion) before being assembled into a full cassette and filled with liquid. This results in a more predictable state of liquid-facing surfaces. Cassettes may be flushed with multiple liquids, maintained at elevated temperatures in the process, and filled with degassed liquids to de-gas components and flush out the majority of any leachable and/or extractable elements.

Sterilization may be performed with liquid in the cassette, and this liquid may be flushed/replaced post-sterilization to remove any byproducts. Coatings or biocoatings may be removed via liquid steps in manufacturing. For example, one or more of the following (alone or in combination) may be used on the cassette or portions of the cassette: coating with a non-stick coating that prevents protein or cell adhesion; coating with films that promote adhesion; coating with extracellular matrix; and removal of temporary coatings (masks) with a solvent liquid to expose regions for cell growth or other coating operations. In some implementations, precise volumes of liquid may be pushed into the cassette in series and then allowed to “incubate” such that different portions of the cassette are exposed to different liquids (for example, to pre-coat specific sections of the cassette with non-stick or adherent films).

All liquid operations may be performed in a sterile environment such as an isolator, after sterilization of the assembled cassette or cassette components. In some implementations, temporary masking layers may be made of temperature-sensitive materials, for example, biocompatible materials that are solid in one temperature range but liquefy in another temperature range. The temporary masks may be used to mask a region (e.g., an adherent cell growth region) during a coating operation and then be removed to expose a certain area, potentially for a second coating operation. Examples of such materials that may be applied, then dried for cassette assembly, then rehydrated, used as a mask, and finally removed via change in temperature while in liquid, are gelatin and polyethylene glycol (PEG), including PEG-PLGA (poly-(DL-lactic acid co-glycolic acid)) copolymers, which can exhibit transitions to liquid at around 37° C.

The final state of the cassette after manufacturing may be liquid-filled, with specific concentrations of ions and dissolved gases, including fully degassed liquid. The liquid may be tailored for optimal long-term storage of coatings or other components within the cassette, including but not limited to ultralow-attachment coatings, adherent coatings, extracellular matrices, laser-activated films, sensor patches such as dissolved oxygen or pH sensor patches, and other components. The cassettes may be sealed in sterile bags or containers that are impermeable to water vapor to minimize evaporation from within the cassette. The cassettes may be shipped and stored at low temperature (for example, 4° C.) to minimize changes in coatings and components.

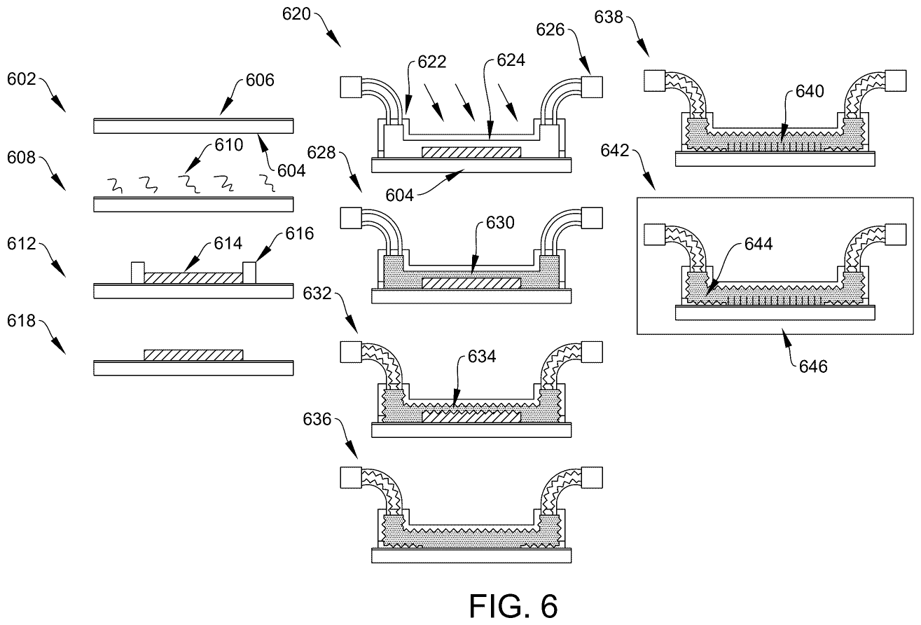

FIG. 6 is a diagram illustrating steps for manufacturing a fluidic cassette in accordance with various implementations. In step 602, the method begins with a glass substrate 604 having a laser-absorbing coating 606 used for cell processing functions. In step 608, a surface preparation 610 is performed prior to coating and adhesion. In step 612, a temporary mask 614 made from biocompatible material (e.g., a PEG-PLGA copolymer that forms a gel at room temperature and goes into solution at >37° C.) is selectively applied on the coating 606 using a gasket 616 or other methods. In step 618, the substrate with the mask is dried.

In step 620, the cassette is assembled, with the substrate 604 and the cassette body 622 forming a fluidic cavity 624. The cassette body 622 may include a gas-permeable top layer opposite the cell growth region on the substrate 604. The cassette may further include fluidic features 626 for exchanging liquids, including scalable ports. Components may be sterilized prior to assembly in a sterile environment, or sterilized as a full assembly (for example, using gamma sterilization), or gamma-sterilized at a later point when liquid-filled. In step 628, the fluidic cavity may be filled with liquid 630. Multiple liquid flushes and steeping steps may be applied to remove any bubbles and foreign particles, and pull the majority of any leachable and/or extractable elements out of the cassette materials. In some implementations, degassed liquid may be used. In step 632, the interior surfaces of the cassette may be coated with an ultralow-attachment film 634 (for example, pHEMA or faCellitate BioFloat™) to prevent attachment of ECM or cells during bioprocessing.

In step 636, elevation of temperature to 37° C. or higher causes the temporary mask formed by PEG-PLGA to go into solution and exposes the substrate in the desired cell growth region. In step 638, an ECM layer 640 (e.g., Laminin, Vitronectin) may be deposited from solution. The ECM layer 640 does not adhere to the ultralow-attachment surfaces 634, and therefore is applied only to the desired growth region. In step 642, the cassette is prepared for shipping and/or storage, filled with a liquid 644 that optimally preserves coating (low attachment and ECM) functionality, and sealed in a sterile container or bag 646. The shipping and storage conditions may be at 4° C. to minimize coating degradation. In some implementations, a low freezing point liquid may be used to maintain cassettes at <0° C. The advantage to the user on the bioprocess end of this cassette is that a simple liquid-to-liquid exchange may be performed to start the process, without worries about bubbles. Moreover, because of the pre-coating of the cassette during manufacturing, no steps must be performed to apply low-attachment and/or ECM coatings as part of the biomanufacturing process.

Cassettes with No-Growth Regions, Designated Growth Zones

In many biological and biomanufacturing applications, it is important to control the spatial growth of cells within a closed cell culture chamber or flow path surfaces. Uncontrolled cell growth can lead to contamination, interference with experimental results, and uncontrolled quality and yield of bioproducts. Thus, there is a need for a system that can precisely prevent cell growth in specific regions while enabling growth only in selected areas. This system must ensure sterility, case of use, and adaptability to various experimental or production environments.

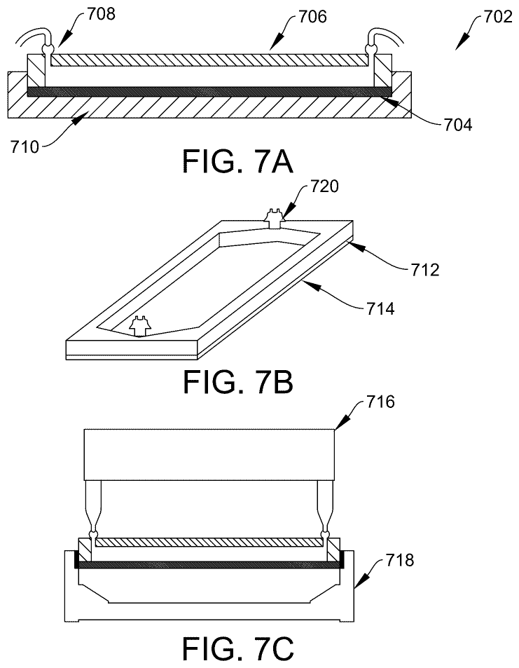

The systems and methods disclosed herein include a cell culture cassette configured with both designated cell growth and non-growth regions. FIGS. 7A-C are diagrams illustrating a cassette for use in a cell culture system in accordance with various implementations. FIG. 7A shows a cell culture chamber 702 that includes a bottom surface 704, a top half-cell 706, and flow path tubing 708 all assembled within an outer frame 710. This design allows for the independent treatment of each region in the cell culture chamber 702 with cell growth inhibitor reagents. As shown in FIG. 7B, the top half-cell 706 and the bottom surface 704 may be adhered together using an adhesive 712 that is protected by a release liner 714. This setup permits the treatment of the top half-cell 706 surfaces with cell growth inhibitor reagents delivered by a liquid handling system 716 or standard laboratory syringes while held in a holder 718, as shown in FIG. 7C. Once treated, the top half-cell 706 may be assembled onto the bottom surface 704 using custom alignment fixtures. Similarly, the flow path tubing 708 may be temporarily shunted before assembly and treated with growth inhibition reagents. Alternatively, the tubing 708 may be treated independently from the half-cell setup. After treatment, the tubing may be cut and connected to the top half-cell barbs 720 as shown in FIG. 7B.

The advantages of the implementation shown in FIGS. 7A-C rely on its architecture and assembly methodology. The design and assembly of the cassette components allow for selective treatment of zones with key reagents to prevent unwanted cell growth in areas outside the designated bottom surface. This enables controlled cell growth in specified regions while ensuring other areas remain free from cell proliferation and attachment. The multiple components facilitate efficient and independent treatment of chamber zones with growth inhibitor reagents, simplifying the process of managing cell growth. The design is flexible and may be adapted for various applications requiring selective cell growth control in closed environments.

### Multi-Port Designs for Flow Control in Fluidic Cassettes

A fluidic cassette system for cell culture may require non-standard fluidic operations to ensure all cell needs are met and enable operations that users need for sampling and treating select zones. Creating complex but well-controlled fluidic paths within a single, closed chamber for cell culture operations and research presents significant challenges. These challenges include ensuring uniform nutrient distribution, precise control over fluid flow, and the ability to perform targeted treatments and sampling without disrupting the overall system. Thus, there is a need in the art for well-controlled fluidic paths within a closed cell culture chamber for cell culture operations and research functions.

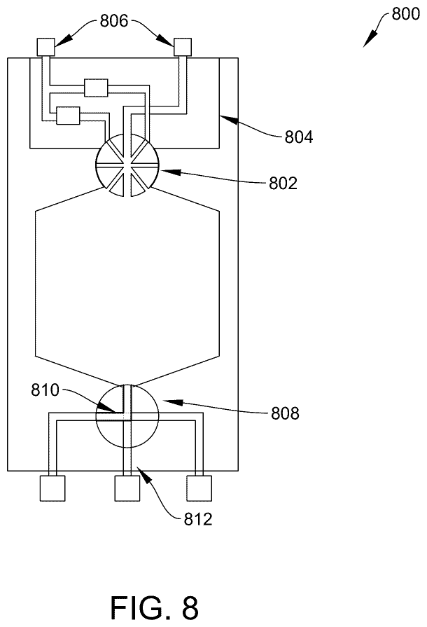

The systems and methods disclosed herein include a fluidic cassette system that incorporates multi-port designs for enhanced flow control. This system enables complex but well-controlled fluidic operations within a single, closed cell culture chamber. FIG. 8 is a diagram illustrating a fluidic cassette 800 for use in a cell culture system in accordance with various implementations. The cassette 800 is the main body of the system and is configured to house the cell culture environment and integrated fluidic paths. The entry port area includes a custom-made rotary modular valve 802 with multiple nozzle configurations used to facilitate precise control of segmented flow paths within the cassette utilizing sheath flow mechanisms. A central flow control manifold 804 allows regulating input fluid flow through the cassette 800 to develop different fluidic resistances using valves and sensors to monitor and adjust flow rates in real-time. The control manifold 804, coupled with the rotary modular valve 802, provides the key control mechanisms for a modular flow path and volumetric flow rates to allow selective flow paths to specific zones in the chamber using only two entry ports 806: the primary liquid and sheathing liquid.

At a fluidic exit path, another rotary valve 808 is utilized to shift between waste and sampling of the reagents. The dedicated sampling tubing path provides a means to extract small volumes of cell culture fluid for analysis and limits dead volume. A waste pathing split 810 may be used to maintain the sterility and integrity of the closed cell culture chamber and sampling collections by integrating a flushing line 812 to prevent debris and waste accumulation. The advantages of the present implementations include allowing uniform reagent distribution and selective zone treatment and sampling within an open cell culture chamber. The open cell culture chamber typically poses challenges in which direct flow segmentation is necessary using physical barriers, but this limits available surface area and cross-communication necessary for multi-cell culture systems. The precise control and fluidic handling modalities provide improved sterility and versatility to support a wide range of fluidic operations tailored to the specific needs of the cell culture environment.

Fluidic Cassettes with High Gas Permeability and Superior Imaging

Performing large-scale automated cell bioprocessing with multiple patient samples in one facility poses many challenges for avoiding cross-contamination during liquid exchanges and incubation. Furthermore, brightfield imaging in typical cell culture formats such as open well plates can create unwanted illumination artifacts due to effects from air-liquid refraction across a curved meniscus and sidewall shadowing. Thus, there is a need in the art for cell culture containers that facilitate high-quality imaging but at the same time prevent contamination of biological samples.

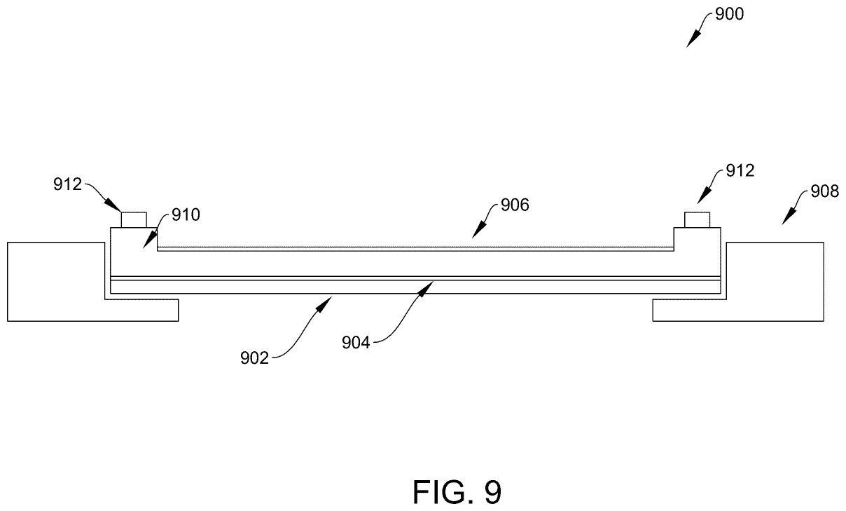

Systems and methods disclosed herein include fluidically sealing each individual cell culture container, and only making fluidic exchanges in dedicated, sterile, patient-specific areas. FIG. 9 is a diagram illustrating a closed cell culture cassette 900 for use in a cell culture system in accordance with various implementations. The cassette 900 includes a transparent substrate 902 (which may be designed with an engineered nanofilm 904 to partially absorb certain laser frequencies) and a gas-permeable and transparent top surface 906, held together in a rigid frame 908. The frame 908 defines the sidewalls of the cell culture chamber and has two or more fluidic entry/exit ports 910. The frame 908 may be constructed from a single part, molded or machined from polymer, metal, or glass, or in other implementations may be a constructed assembly of multiple parts. Its fluidic function is to define the overall height of the liquid volume and to shape the flow profile of liquid during fluidic transfer. It can also be designed to interface with fluidic connectors for aseptic fluid exchange as well as to physically couple to other instruments.

The gas-permeable and transparent top surface 906 may be made of a polymer material through which certain gases such as oxygen and carbon dioxide are able to permeate, but would also be sufficiently impermeable to water vapor such that evaporation would not occur. Materials for such a top surface that provides some gas permeability but also good optical clarity include, but are not limited to, cyclo olefin polymer or co-polymer (COP, COC), polymethylpentene (PMP), low-density polyethylene (LDPE), polystyrene, Teflon AF, or other fluoropolymers. The top surface 906 may or may not be flexible. The cassette 900 may be designed with a low aspect ratio to minimize sidewall shadows, and with a flat top surface 906 to improve illumination uniformity.

### Washing Methods in a Cassette Using Gas-Liquid Interfaces

To remove debris and dead cells from an adherent culture in a closed fluidic format such as a cassette, shear stresses resulting from flow of liquid between the inlet and outlet port may be used to lift any partially adhered material. However, the strength of an induced shear stress is directly related to the velocity of the flow, which is highly geometry dependent and may become negligible in large cross-sectional formats. Thus there is a need in the art for reliable, uniform fluid flows in low profile cassette formats.

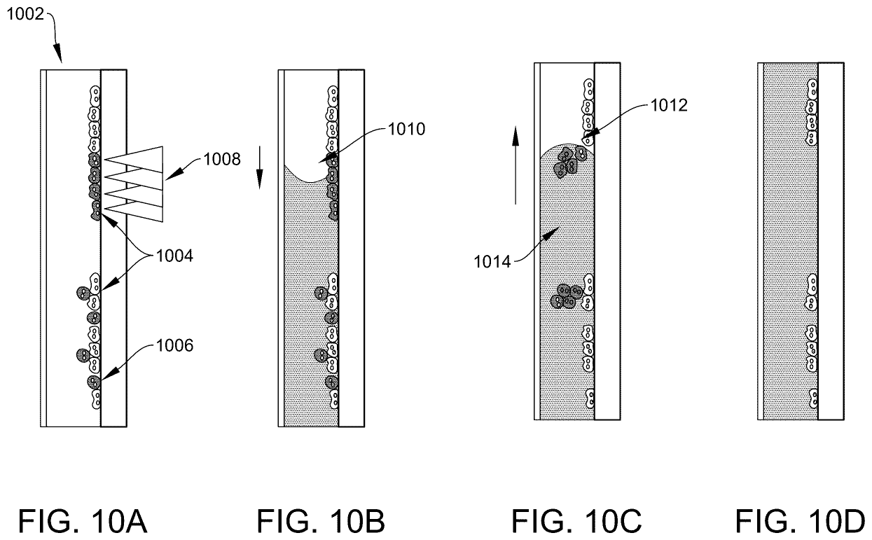

Systems and methods disclosed herein include introducing a controllable detachment force to any particles on the cell culture surface of a fluidic cassette by using a liquid-gas interface. FIGS. 10A-D are diagrams illustrating a method of fluid washing in a closed fluidic cassette in accordance with various implementations. FIG. 10A shows a fluidic cassette 1002 containing an adherent cell culture 1004. Within the cell culture 1004 there may be dead cells or debris 1006 or cells that have been selectively killed and/or dislodged using laser scanning 1008. In FIG. 10B, the cassette 1002 may be oriented vertically (or at some angle above horizontal) and a gas may be flowed through the upper fluidic port, which will result in a flat liquid-gas meniscus 1010 that may be swept across the cell culture. This phase interface imparts a normal force to any particle on the surface, the magnitude of which depends on the surface tensions between the liquid-surface interface, the liquid-particle interface, and the direction of flow (advancing or receding), all of which can be tuned for a given washing process. Other factors that influence the lifting force include the angle of the cassette with respect to gravity, the vertical height of the liquid, the number of cycles, and the flow rate. These factors may all be controlled to further optimize the effectiveness of the washing. For example, FIG. 10C shows an advancing interface 1012 with a liquid 1014 that is potentially different from typical cell culture media that could be used to provide sufficient lifting force to remove the unwanted dead cells and debris for continued cell culture without removing, or with limited removal of, cells of interest (as shown in FIG. 10D).

### Dynamic Flow Shaping in a Cassette

To wash debris and dead cells from an adherent culture in a closed cassette, shear stresses resulting from flow of liquid between the inlet and outlet port can be used to lift any partially adhered material. However, the strength of an induced shear stress is directly proportional to the gradient of the velocity of the flow, which can vary along the culture surface, depending on the cross-sectional geometry, resulting in non-uniform washing. Thus there is a need in the art for reliable, uniform fluid flows in low profile cassette formats.

Systems and methods disclosed herein include cell culture cassettes configured such that the top surface is flexible and so can be set to obtain a particular shape based on the relative pressure difference between the surrounding ambient environment and interior liquid. This shape may then be used to tune the liquid velocity profile through the cell culture flow chamber resulting from flow induced by a pressure differential between the fluidic entry port(s) and fluidic exit port(s). This shape may be dynamically adjustable during the course of flow for a variety of liquid exchange operations.

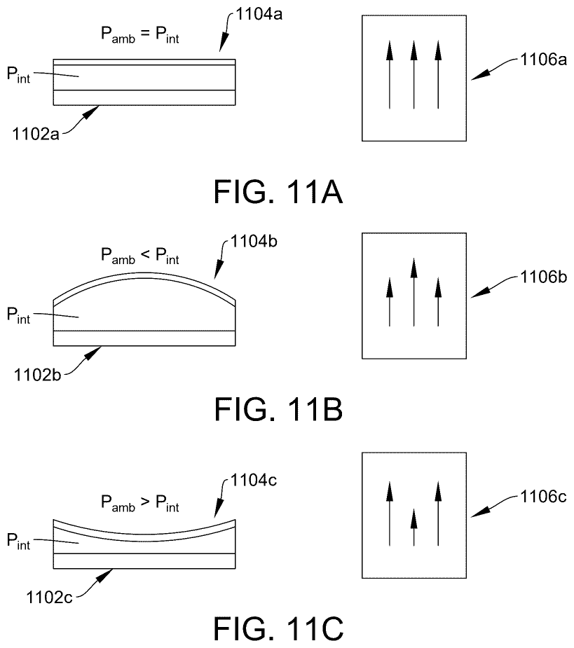

FIGS. 11A-C are diagrams illustrating another method of fluid washing in a closed fluidic cassette in accordance with various implementations. FIGS. 11A-C show cell culture surfaces 1102a, 1102b, and 1102c respectively. To increase or decrease the shear stress magnitude at different locations along the width of the cell culture surfaces 1102a, 1102b, 1102c, cross-sectional shaping of top surfaces 1104a, 1104b, 1104c of the cell culture chamber may be used to increase flow velocity either towards the center or the edges of the cell culture chamber by adjusting the net pressure difference between the inside of the chamber (Pᵢₙₜ) and ambient (P_{amb}) and deforming the flexible top surfaces 1104a, 1104b, 1104c.

FIG. 11A shows the case when P_{amb}=Pᵢₙₜ, in which the top surface 1104a is flat and the velocity variation across the width of the cell culture chamber will not experience any additional variation from edge to edge, as shown by flow diagram 1106a. FIG. 11B shows the case when P_{amb}<Pᵢₙₜ, in which the top surface 1104b expands upwards, increasing the cross-sectional height more in the center of the cell culture chamber as compared to the edges. This will result in an effect to the velocity profile that favors faster flow (and therefore greater shear) along the center, as shown by flow diagram 1106b. FIG. 11C shows the case when P_{amb}>Pᵢₙₜ, in which the top surface 1104c is pulled inwards, decreasing the cross-sectional height more in the center of the cell culture chamber as compared to the edges. This will result in an effect to the velocity profile that favors faster flow (and therefore greater shear) along the edges, as shown by flow diagram 1106c. By alternating between these top surface configurations, one can ensure sufficient shear for washing across all areas of the cell culture surface.

### Density-Based Liquid Replacement in a Cassette

When trying to displace a liquid with an incoming liquid in a closed fluidic container, efficient replacement may only be possible with excessive volume flowed through, or with very small fluidic cross sections. This operation becomes even more difficult if the two liquids have similar properties (e.g., same temperature, density, viscosity). This type of operation is required during cell media changes, which occur very frequently during any type of cell culture. Thus there is a need in the art for efficient methods of liquid replacement in closed cassette cell culturing formats.

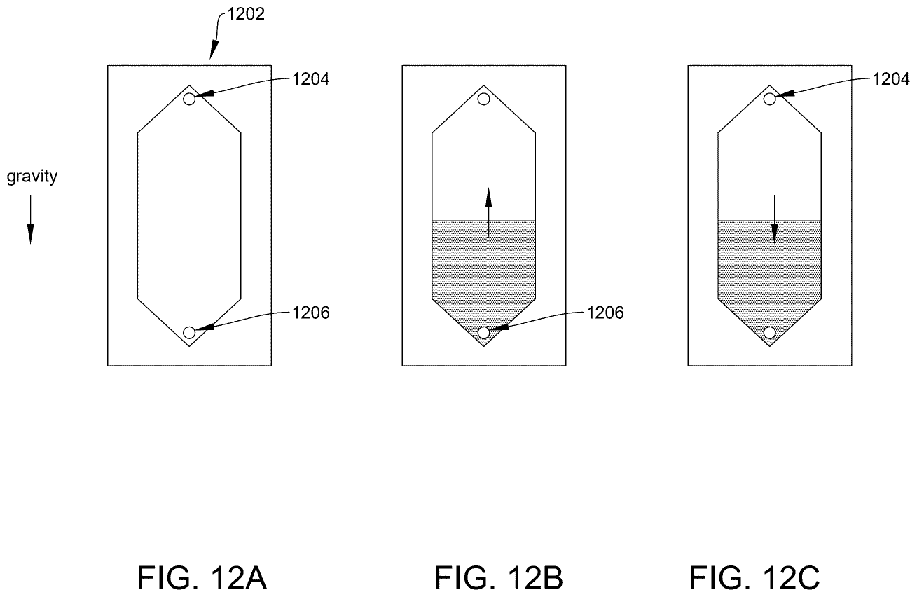

Systems and methods disclosed herein include methods for density-based liquid replacement in a cell culture cassette format. FIGS. 12A-C are diagrams illustrating a method for liquid replacement in a closed cassette in accordance with various implementations. FIG. 12A shows a cell culture cassette 1202 oriented such that the fluidic entry point 1204 and exit point 1206 are at different heights. Density-based separation of sequentially flowed fluids may be used to carefully control the distribution or removal of biological material (such as cells, ECM, growth factors), cell culture nutrients, gas, dissociation reagents, or balanced salt buffers, regardless of the chamber geometry. FIG. 12B shows the case in which the liquid entering the cell culture chamber is denser than the fluid to be displaced, and so the flow is introduced from the bottom port 1206. FIG. 12C shows the case in which the liquid entering the cell culture chamber is less dense than the fluid to be displaced, and so the flow is introduced from the top port 1204. This method of liquid replacement may be used in any cell culture chamber shape, provided the flow rates are slow enough to not introduce turbulent mixing.

In implementations in which the incoming liquid (e.g., cell culture media) has very similar density as compared to the liquid to be replaced, an intermediate liquid with a different density may be used before introducing the new media. In this implementation, the cell culture cassette 1202 may be oriented to a vertical position, and a low density intermediate (for example) would be introduced from the top fluidic port 1204, displacing the higher density media towards the bottom. Subsequently, the higher density, new media would be introduced from the bottom port 1206, displacing the low density intermediate solution out of the upper port 1204. Density of liquids may be controlled to achieve these exchanges using minor tuning of salinity, of other constituent concentrations, or of temperature.

Actuatable Bubble Reservoirs for on-Board Mixing/Washing

When operating a closed cell culture chamber, there are limitations in how fluid may be added. Typically this is done with inlet/outlet ports. However, the consequence of having relatively small ports compared to the overall cell culture chamber size is the reduced efficacy in removing debris and particles not adhered to the extracellular matrix. Thus there is a need in the art for more effective mixing and washing operations in a closed cassette format.

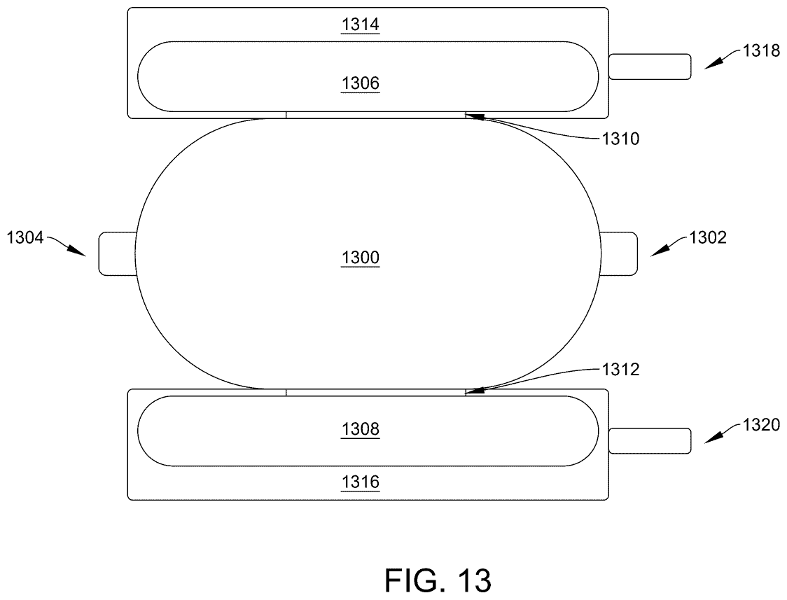

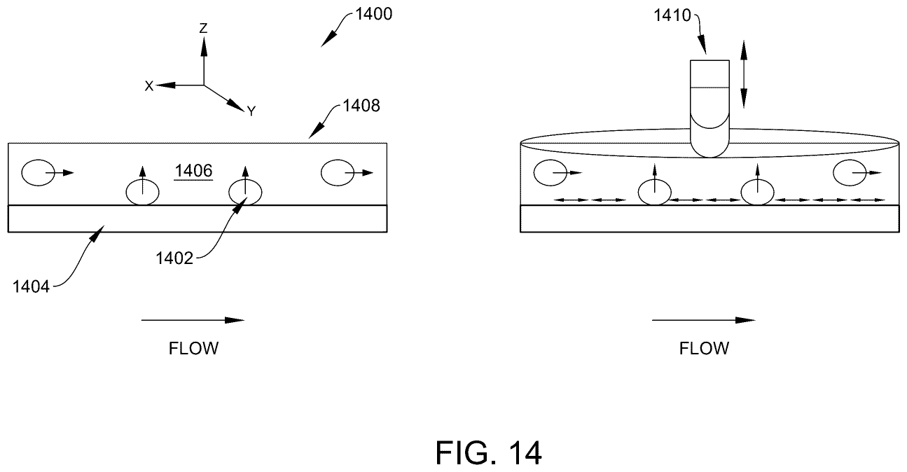

Systems and methods disclosed herein include bubble reservoirs configured to enable on-board mixing and washing operations of closed cell culture cassettes. FIG. 13 is a diagram illustrating a cell culture chamber 1300 in accordance with various implementations. The cell culture chamber 1300 includes an inlet 1302 and an outlet 1304. Flexible chambers 1306, 1308 are connected to the cell culture chamber 1300 via passthroughs 1310 and 1312, respectively. The flexible chambers 1306, 1308 are housed in sealed compartments 1314, 1316, respectively, which are in turn connected to pressure sources via ports 1318 and 1320, respectively.

To introduce fluids into the cell culture chamber 1300, both sealed compartments 1314, 1316 are pressurized to compress the flexible chambers 1306, 1308. Fluid is introduced into the cell culture chamber 1300 through the inlet port 1302, with any fluid exceeding the volume of the cell culture chamber 1300 leaving through the outlet port 1304. Once the cell culture chamber 1300 is filled, pressure is relieved from sealed compartment 1314, and fluid enters into flexible chamber 1306. Fluid into inlet port 1302 is stopped once the flexible chamber 1306 reaches a desired capacity. Both ports 1302, 1304 are then sealed.

To mix fluids in the cell culture chamber 1300, pressure is relieved from the sealed compartment 1316. Pressure is then applied to sealed compartment 1314 and fluid from the flexible chamber 1306 is allowed to enter into the cell culture chamber 1300 and eventually fill flexible chamber 1308. Once chamber 1308 is filled, the process is reversed to send fluid from chamber 1308 to chamber 1306. This cycle may be repeated until sufficient mixing and/or washing of the cell culture surface is achieved.

To purge fluids from the cell culture chamber 1300, ports 1302, 1304 are opened and fluid is allowed to enter through inlet port 1302. Both sealed compartments 1314, 1316 are pressurized to collapse both flexible chambers 1306, 1308, and the debris-filled fluid is allowed to exit through outlet port 1304. After a period of time, pressure is relieved from sealed compartment 1314 and fluid enters into flexible chamber 1306. Fluid into inlet port 1302 is stopped once the flexible chamber 1306 reaches desired capacity. Both ports 1302, 1304 are then sealed.

Automated Shear Flow Operations Combined with Mechanical Actuations

Maintaining cell cultures in closed fluidic environments often requires making use of fluidic shear forces to detach adherent cells, debris, or bubbles from surfaces. However, in geometries with large cross-sectional areas, shear forces can become vanishingly low. Relying on shear stress alone may not be sufficient to achieve the necessary detachment forces in a cell culture system. Thus, there is a need in the art to improve fluid flows in closed cell culture formats for the purposes of detaching cells.

Systems and methods disclosed herein include using external mechanical actuation in addition to fluid flows to enable effective cell culture clearing operations. FIG. 14 is a diagram illustrating a fluidic cell culture chamber 1400 in accordance with various implementations. The cell culture chamber 1400 includes cells 1402 (which may be adherent or non-adherent) on a cell culture surface 1404 within a liquid culture medium 1406, which is fluidically contained by a top surface 1408, which is made of a flexible or compliant material that may be manipulated (e.g., bent) by mechanical actuation. During flow, liquid will impart a shear stress onto the top and bottom surfaces of the cell culture chamber 1400.

Before, during, or after this flow, additional mechanical forces 1410 may be applied to the fluidic container which further help in detaching stuck particles from the surface. The particles may be adherent cells, debris, or gas bubbles. The nature of the mechanical forces 1410 may be accomplished using, for example, mechanical tapping (periodically spaced impulses), vibrations (e.g., higher frequency movements, for example with a frequency on the order of 10 kHz) on the rigid cell culture surface 1404 or some other surface rigidly attached to the cell surface along the axes X, Y, or Z, or a physical displacement of the top surface 1408 (which may be flexible), imparting transient pressure changes in the fluidic environment. For example, tapping of the top surface 1408 introduces local variations in the liquid pressure which may aid in particle detachment. In some implementations, the orientation of the cell culture chamber 1400 may be arranged such that the fluidic exit port is above the fluidic entry port, facilitating the clearance of gas bubbles.

### Non-Pulsatile Peristaltic Feeding and Washing System for Closed Cassettes

Peristaltic-driven flow in bioprocessing systems has the advantage of being completely sanitary and non-contacting. However, given the pulsatile nature of typical circulating rollers in a peristaltic pump, the resulting flow rate through an adherent cell culture container can be oscillatory, which may produce an unwanted effect on the sensitive cells. A separate challenge in automated cell culture is the equilibration of cell culture media from 4° C. storage temperature to a raised process temperature, before introducing it into the cell culture container. In addition to temperature, certain gas concentrations may also need to be established. Running an aliquot equilibration step for a cell culture container while maintaining sterility can often be a challenge in automated systems. Thus, there is a need in the art for applying media operations in a closed cell culture container format while maintaining sterility.

Systems and methods disclosed herein utilize a relatively large diameter section of tubing that serves as a pre-feed equilibration reservoir as well as a displaceable volume for media changes for a closed cell culture cassette, instead of a traditional section of tubing that fits into a rotating peristaltic pump. Instead of a rubber stopper getting pushed down a column like in a syringe, the large diameter tube is squeezed down by rollers. By straightening out this peristaltic pump, there is much better control of the volumetric flow rate, and no pulsing or backflow, and it would simultaneously serve as an equilibration reservoir if made out of a gas-permeable, elastomeric material such as silicone.

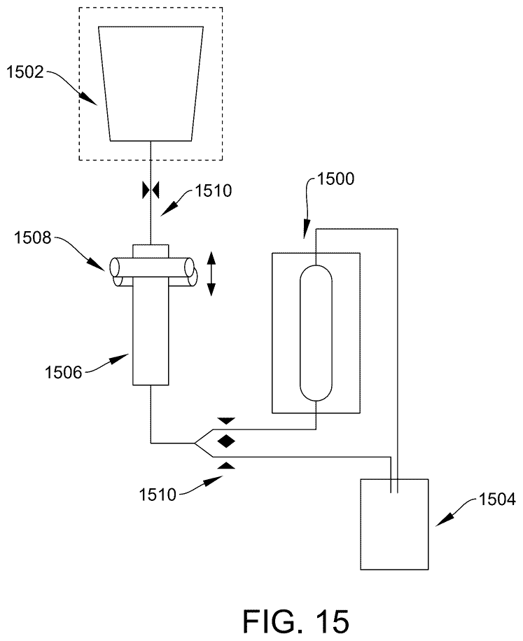

FIG. 15 is a diagram illustrating a closed cell culture container 1500 (e.g., a cassette) and a pump system in accordance with various implementations. An inlet port of the cell culture container 1500 is attached to tubing which is fed by an upstream media storage bag 1502 held at 4° C. An outlet port of the cell culture container 1500 is attached to a sterile waste container 1504. Between the storage bag 1502 and the cell culture container 1500 is a larger diameter tubing section 1506 that may be squeezed by a pair of actuated rollers 1508. By translating the rollers 1508 downwards, the media in the large diameter tubing section 1506 is pumped into the cell culture container 1500 with very good control over a wide range of flow rates. Furthermore, if the material of the large diameter tubing section 1506 is temperature controlled and gas-permeable, then the media may be equilibrated as needed before introduction into the cell culture. This tubing system could also have a set of independently actuated pinch valves 1510 to prevent unwanted pulling of storage media into the equilibration volume or to switch between feeding the cells or going directly to waste (for example, to prime out old media or bubbles).

### Adhesion Reduction Factor-Accelerated Laser Cell Removal

It has been shown that pulsed laser treatment of cell culture surfaces may be used to selectively kill and remove portions of colonies on a growth surface. To achieve good clearing of laser-scanned cells, a post-scanning wash is often required, which detaches and removes the dead cells using shear forces from liquid flow. However, this washing can sometimes negatively affect healthy, unscanned colonies by detaching cells that are more weakly attached to the surface. It is therefore important to ensure that the cells that have been laser scanned are significantly less adhered to the surface than any healthy cell that is intended to be kept. Thus, there is a need in the art for methods to control the adhesion of cells that should be removed from a cell culture surface versus cells that should remain.

Systems and methods disclosed herein include applying a chemical treatment to a cell culture to reduce adhesion for cells that should be removed from the cell culture. After laser scanning of a cell culture and before washing, a short chemical treatment may be applied to the entire cell growth region which selectively targets cells that have been affected by the laser. The chemical treatment lowers the force of adhesion of these scanned cells even more as compared to unscanned cells, allowing for a more gentle washing step that does not result in off-target washing of healthy cells.

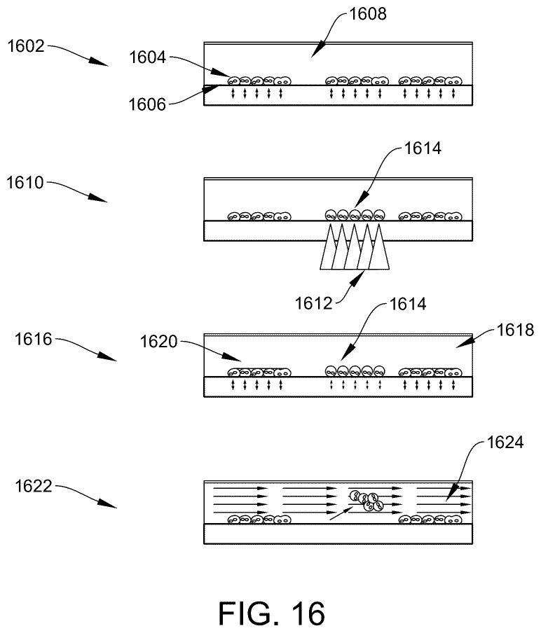

FIG. 16 illustrates a method of applying adhesion reduction to a cell culture in accordance with various implementations. In step 1602, cell colonies 1604 are adhered onto a cell culture surface with a laser-absorbing thin film 1606 inside a fluidic cell culture container 1608. The cells in the cell colonies 1604 all have roughly equal magnitudes of adhesive interactions with the surface. In step 1610, short laser pulses 1612 are applied to the cell culture to selectively kill targeted cells. After the laser treatment, some scanned cells 1614 may be dead but still physically adhered to the cell culture surface. In many cases, the scanned cells 1614 begin to detach from their neighboring cells, exposing more membrane to the surrounding fluid. In step 1616, a liquid-based chemical 1618 may be introduced into the cell culture container 1608. The liquid-based chemical 1618 will have greater areal exposure to the cell membranes of the scanned cells 1614 as compared to healthy unscanned cells 1620 due to the latter being more tightly packed together. Using a chemical or solution which is known to reduce adherent cell binding to surfaces (such as protein-cleaving enzymes like trypsin, calcium chelators like EDTA, or phosphate-buffered saline), the attachment force of the scanned cells 1614 may be reduced and then more easily wash them away in a subsequent fluid flow 1624, shown in step 1622.

### Fluidic Cassette Designs for Long Term Adherent Cell Cultures

There is a need in the art for improved designs in closed cell culture formats (e.g., closed cassettes) that facilitate healthy maintenance of long term adherent cell cultures. The systems and methods disclosed herein include fluidic cassette formats that enable long term (e.g., 60 days or longer) adherent cell cultures. In some implementations, the fluidic cassette includes a transparent surface for the coating, seeding, growth, imaging, and laser scanning of adherent cells. The transparent surface may be, for example, glass or fused silica. In some implementations, the transparent surface is coated with a light-absorbing film (e.g., inorganic) that is configured to withstand temperature spikes (e.g., 1000+Kelvin) when impinged upon by laser light. In some implementations, the film is thin (e.g., roughly 100 nanometers) and has a strong optical absorption at certain wavelengths (e.g., high optical extinction coefficient, such as 30%). In some implementations, the film is configured to allow cell adhesion molecules to bind onto a film, such as nanometer-thick ECM. The ECM should be thick enough so that the laser light is not significantly absorbed by it.

In some implementations, the film is configured to allow for direct bonding to a structure (e.g., by tape or other means) so that laser scanning may take place at the edge of a cell culture chamber. Furthermore, due to intrinsic uncertainties in controlling the laser spot, the film may enable scanning of film regions that are directly bonded to the structure without weakening or damaging the bond, and without producing cytotoxic chemicals. In some implementations, the film is configured to support high shear stress so that the cells may be washed away. Significant dissolution of the film by shear stress would result in lost functionality of the film, making it less effective for supporting long term cell cultures. In some implementations, the cassette and the film may be subjected to pressures on the order of two atmospheres (c.a. 30 psi) during routine washing steps, so film porosity should be minimized. Pores that shatter during routine pressurized use would accelerate dissolution. The bond between the film and the structure should not weaken due to exposure to stress and liquid. Thus, the film is configured to resist dissolution during long term cell cultures, since dissolution could lead to accelerated separation of the bond between the film and the structure.

In some implementations, the dimensions of the structure should be chosen to facilitate good imaging (e.g., sufficiently thin) yet be rigid (e.g., sufficiently thick) to not deform when exposed to high pressure. The dimensions of the cell culture chamber should be chosen to facilitate good gas exchange (e.g., sufficiently thin) yet be rigid (e.g., sufficiently thick) to not deform when exposed to high pressure. For example, the thickness may range from 25-500 microns, depending on the properties of the material used to construct the chamber (e.g., permeability to oxygen and carbon dioxide). In one example in which a rigid roof structure is desired, the thickness of the chamber may range from 250-500 microns. In another example in which a flexible roof structure is desired, the thickness of the chamber may range from 25-150 microns. Gas permeability promotes good cell growth in a CO₂ or O₂ incubator. Since gas permeability through glass or fused silica is insignificant, gas exchange should take place through the chamber, which is typically manufactured with plastic. Certain newer plastics, such as polymethylpentene, exhibit sufficient permeability to satisfy these two conditions. In some implementations, the structure is constructed of a plastic or glass. Finally, the thickness of the structure must not create a distortion in the illumination such that automated image-based analysis is impaired.

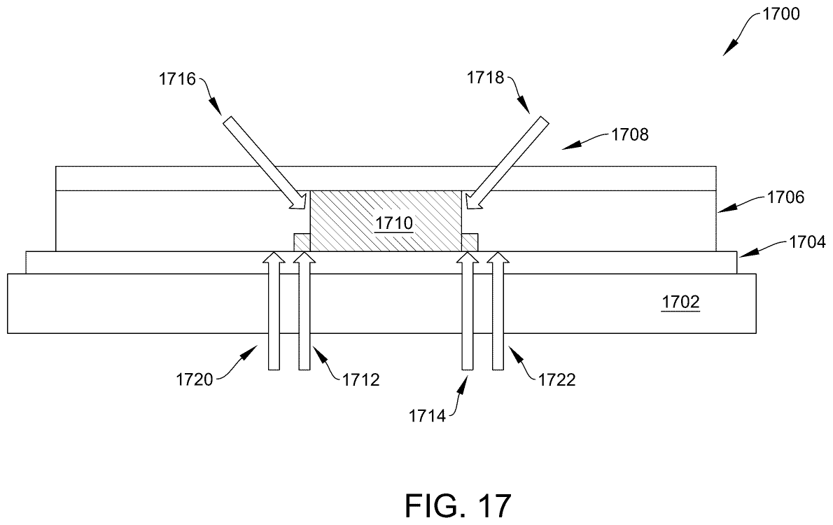

FIG. 17 is a diagram illustrating a fluidic cell culture cassette 1700 in accordance with various implementations. The cassette 1700 includes a substrate 1702 that is coated with a laser film 1704, upon which a structure 1706 is applied. Structures 1706 and 1708 (typically made from glass or plastic) may be used to constrain liquid to a region 1710, which is typically filled with cell culture media. Cells growing in regions 1712, 1714 (near the undercut boundaries that constrain the liquid) have a tendency to grow up walls 1716, 1718 respectively. However, the film 1704 enables the laser to scan regions 1712, 1714, and even under the structure 1706 in regions 1720 and 1722, so can prevent growth of cells up the walls 1716, 1718. As disclosed herein, the film 1704 is configured to resist dissolution so that the bond between the film 1704 and the structure 1706 is maintained, in spite of the internal pressures during flow of liquid in the region 1710.

### Mechanical Actuation Methods for Liquid and Particle Management in a Closed Cassette

Maintaining a cell culture in a closed fluidic container often limits the type of flow that can be obtained through just a pair of inlet and outlet ports. This can lead to fluidic challenges for operations like liquid replacement, homogenization of suspended particles in solution (such as cells), removing bubbles, and applying shear forces for lifting. Thus, there is a need in the art for improved fluidic management and operation in a closed cell culture container format.

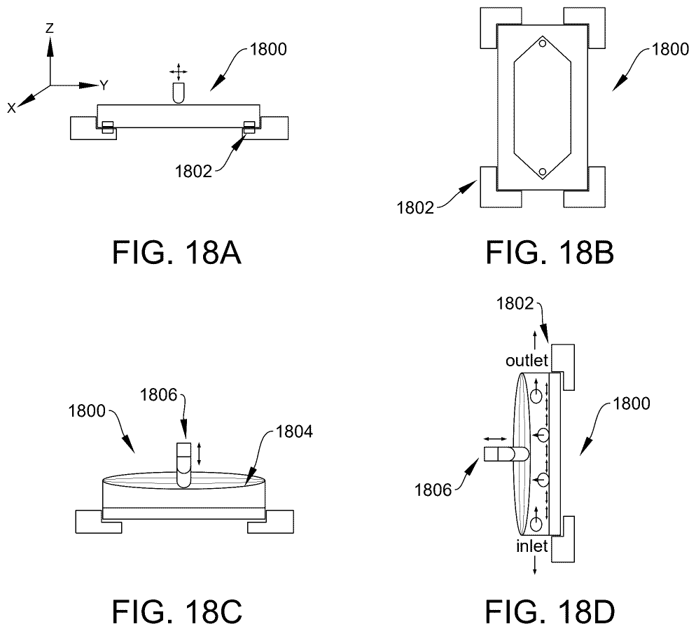

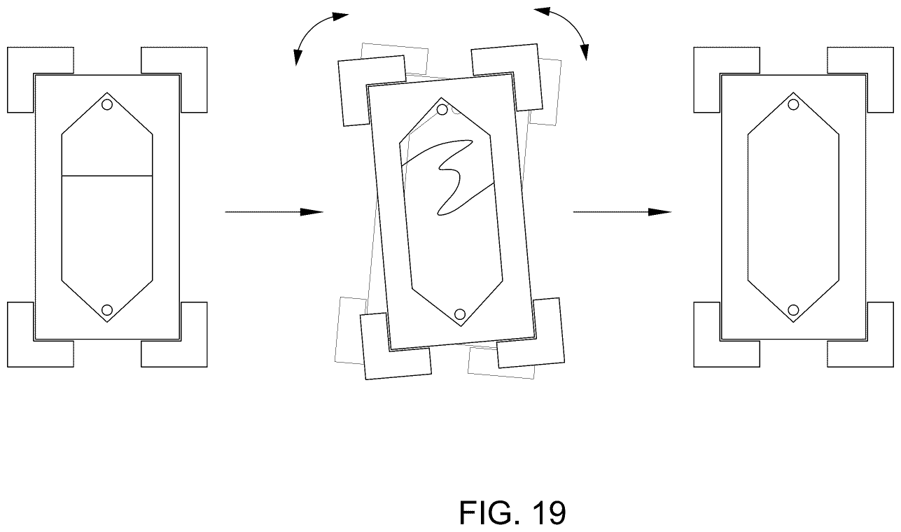

Systems and methods disclosed herein include designs for a cell culture container and frame within which it is fastened that allows various mechanical forces to be externally applied. FIGS. 18A-D are diagrams illustrating various cell culture containers and frames in accordance with various implementations. FIG. 18A shows a closed cell culture container 1800 from a side view, while FIG. 18B shows the cell culture container 1800 from a top view. The cell culture container 1800 is immobilized in a movable nest 1802. The movable nest 1802 may be designed to accept a cassette or other cell culture container, lock it into place (for example, using electromagnetics), and then position the cassette linearly or rotationally along any axis in addition to providing higher frequency actuation such as tapping, shaking, or vibrating.

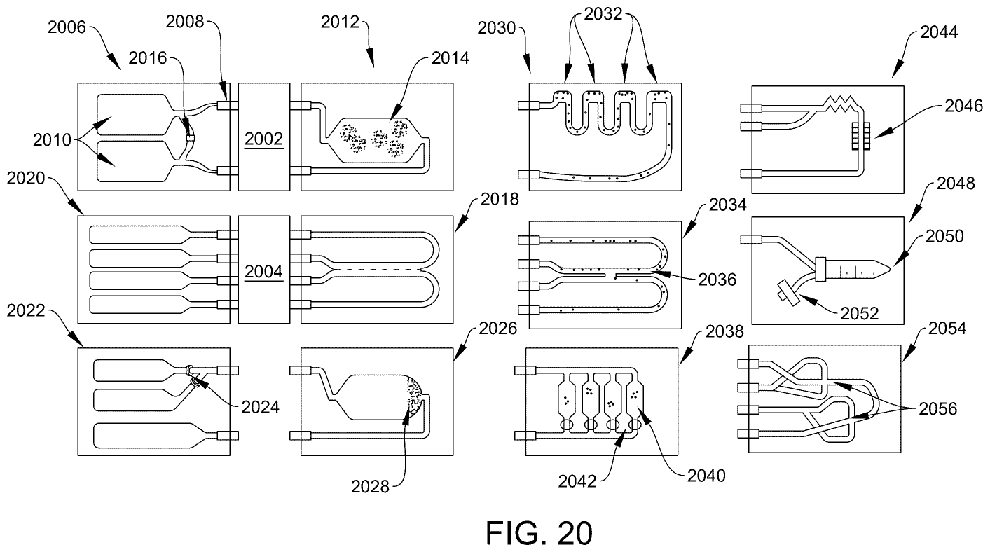

In some implementations as depicted in FIG. 18C, the cell culture container 1800 may include a flexible top surface 1804. Additional forces may be applied to the top side of the cell culture container 1800 by a movable actuator 1806 that also translates perpendicular to the cell culture container 1800. In other implementations, an actuator may induce rapid pressure changes in the ambient air outside of the cell culture container 1800, which causes displacement of the flexible top surface 1804. FIG. 18D shows the cell culture container 1800 during fluid flow, in which the movable nest 1802 may orient the cell culture container 1800 vertically such that air bubbles travel upwards towards the outlet port. Before, during, or after flow, agitation of the cell culture container 1800 roof via the movable actuator 1806 may produce alternating shear forces within the fluid and help to dislodge pinned bubbles. This operation may also be used to apply shear forces to anything in general, including cells during lifting steps, for example. FIG. 19 is a diagram illustrating applying rotational motion on a closed cell culture container (e.g., a cassette) in accordance with various implementations. The rotational motion may result in the mixing of two or more liquids, or uniform dispersal of particles in solution such as cells or other biological materials. Such rotational motion may be accomplished by a number of configurations of automated equipment, including but not limited to rotational stages into which the cassette is placed, or a robotic arm. The rotational mixing may be performed during a liquid exchange operation.

### Pluggable Functional Cassette-Based Cell Processing Systems

Traditional laboratory practices in which cell processing instruments such as centrifuges, cell sorters, electroporators, etc. are shared among biological samples do not extend to good manufacturing practice (GMP) manufacturing of cells and cell products, in which cell-related materials must be strictly isolated from contamination sources, and different batches of cells (for example, patient batches) must also be strictly isolated from one another to prevent crossovers or cross-contamination. Thus, multiple architectures have been developed for batch-isolated cell processing. A common (unsolved) problem of all these architectures is balancing complexity and cost against flexibility, both in terms of the range of bioprocesses that a particular system or system configuration can accomplish, and the range of cell products that may be produced.

The first attempts at solving this GMP sterility/batch isolation problem are usually made by simply performing all operations in isolated environments, for example, in separate cleanrooms, with any open steps performed in a biosafety cabinet. In other cases, isolator systems are utilized. The problems with these systems include, for example, a large amount of manual work, requiring significant gowning/de-gowning for cleanroom entry, large footprints, extensive decontamination work between runs, and high expense. The use of laboratory-style equipment such as centrifuges, cell sorters, electroporators, and microscopes entails painstaking sterilization of surfaces before the equipment may be used for another cell batch.

A next set of attempts to run complex, small-batch patient cell processes revolved around one-time use tubing sets with one-time use components attached to them. Some prior art approaches use a large array of pinch valve actuators, and positions for bioprocess system components such as magnetic bead sorter, growth chamber, and centrifugation. Bags with supplies/reagents and cells, as well as bags for harvesting, or other cell culture containers, must be tube welded onto the device. Moreover, provisions for using external instruments such as microscopes or incubators for attached cell culture chambers are not made—the tubing must stay attached at all times.

Other attempts to solve the problem of running complex cell processes in small batches, specifically for autologous medicines, have centered around one-time use modules. The approach of these systems has been to encapsulate all possible bioprocess steps and functionalities into a single, one-time-use module. This reduces the number of setup, resupply, and harvesting operations that must be manually performed by an operator. However, to achieve maximum process flexibility, a large number of subsystems, tubing arrays, fluidic switching, and ports must be integrated on the module, making it voluminous, complex, and expensive. Thus, there is a need in the art for compact, easy to use and maintain multi-batch cell culture systems.

Systems and methods disclosed herein include a system for bioprocessing that includes a series of fluidic cassettes that are function-specific, and are capable of being interfaced with other cassettes through a coupling system, which may be an aseptic coupling system. Cassettes may be coupled together or used independently for a single operation or series of operations, on one or more stations corresponding to the operation and cassette types in use. This allows for a highly flexible bioprocessing system capable of running a range of processes, or multiple batches at different points in their processes. Moreover, it allows a flexible and efficient configuration of equipment to reduce bottlenecks and make optimally efficient use of capital equipment. The system is highly compatible with automation, including automated fluidic coupling, transport, and unit operations using functional cassettes. The system employs cassettes that are extremely versatile in terms of their functional payload, with much of the mechanical portion of the cassettes reusable.

FIG. 20 is a diagram illustrating a modular bioprocessing system that uses a plurality of cassettes in accordance with various implementations. FIG. 20 depicts an example, non-exhaustive array of functional cassettes, which may be connected via cassette coupling systems 2002, 2004 configured to couple multiple fluidic/gas transfer ports between cassettes. The coupling system 2002, 2004 may be aseptic in nature and are configured to enable cassette-to-cassette interconnections during the performance of cell processing functions. Cassette 2006 shows an example cassette for fluidic supply/waste, or fluidic mixing operations. All cassettes in the example shown in FIG. 20 include pluggable ports 2008 that are normally closed, except when connected. These ports may further be aseptic. Cassette 2006 features two liquid storage compartments 2010. Liquid may be transferred to/from these compartments 2010 by use of an external liquid transfer handler. The handler may, for example, apply mechanical pressure to one of the compartments (such as from a roller that traverses it as described herein) to push fluid out, apply air pressure through a sterile filter to push fluid out, or use a peristaltic pump on tubing exposed on the cassette to push fluid out of the compartment.

In the example shown in FIG. 20, the fresh media may be pushed through the aseptic coupler 2002 into a cell culture cassette 2012 with a cell growth chamber 2014 used to change cell media. The growth chamber 2014 may include a laser-activated film to allow laser cell deletion or intracellular delivery via microbubble formation and collapse. Another example feature on the fluidic supply cassette 2006 is a bypass valve 2016, which may be opened by an external fluid management system via mechanical or electromagnetic means. The bypass valve 2016 allows flow between the fluidic compartments 2010, for example, for a mixing operation. In other implementations, a media management cassette that is attached to a cell growth cassette may circulate media either in continuous-flow or stopped-flow modes through the growth cassette via peristaltic or other pumping. Such a management cassette may also incorporate features for managing dissolved gases (for example, gas exchange sections), sensors for monitoring media status, the ability to supplement media with various factors, and other components.

Another example of cassettes possible in the system described herein is cassette 2018, which is a cassette for tangential flow filtration operations (in which flow along a semiporous membrane allows for transfer of certain constituents from one flow to another). The cassette 2018 is shown coupled via coupler 2004 to a different fluidic supply cassette 2020 with four fluidic compartments. The coupled cassette assembly (cassettes 2018, 2020 and coupler 2004) may be transported to a station specifically designed to perform the filtration operation, with fluidic reservoir actuators, sensors, and other features useful for the operation.

Cassette 2022 is an example of another fluidic supply cassette, wherein one port is attached to two reservoirs, with source selection performed by two pinch valves 2024 actuated by an external device. For example, one reservoir may contain fresh cell media and another reservoir may contain washing liquid such as phosphate-buffered saline (PBS). In another example, one reservoir may contain cell dissociation agent, and the other reservoir may contain a buffer for cell harvest. The reservoir connected to the other port may be a waste or cell collection reservoir.

Cassette 2026 shows a configuration that may be used for centrifugation, or counterflow centrifugation. As the cassette 2026 is rotated off-center, cell or other material accumulates at one end 2028 of the chamber. If the cassette 2026 is connected to another cassette (or through the use of on-cassette reservoirs), a counterflow may be applied that acts in the opposite direction as the centrifugal forces. This is an effective means of separating particles based on density and size. The particles may then be harvested directionally (towards either port) according to sort order. Alternatively, the particles may be processed/deleted/disabled via laser irradiation through the cassette window, for example, to kill a subpopulation of cells that have been counterflow centrifuged.

Cassette 2030 shown in FIG. 20 is configured for magnetic bead sorting. Cassette 2030 has a channel that is curved to provide mixing or flow reconfiguration, and a series of surfaces 2032 where an external magnetic field may be applied (for example, using rare earth magnets that reside on an external device) to temporarily immobilize cells that are attached to paramagnetic beads along the surfaces 2032. The flow may be repeated in forward and reverse directions from a coupled source cassette. Finally, the cassette 2032 may be re-coupled to a second destination cassette, the external magnetic field removed, and the bead-attached cells harvested (in a positive-selection scenario; a negative-selection scenario uses a different set of steps).

Cassette 2034 shown in FIG. 20 is configured for cell sorting using a microfluidic cell sorter. Cell sorting may be accomplished, for example, by using a magnetic switching element actuated by an external electromagnet. In other examples, cell sorting may be conducted using a bubble-driven cell switch driven by external electrical currents, or laser pulses to enable cells in a flow to be switched at a junction 2036 based on optical observations made in the upstream flow. The cell sorter cassette 2034 may be attached for the duration of the operation to another fluidic cassette with source cells, sheath media if needed, collected cell reservoir, and discarded cells/media. A passively switched/sorted fluidic configuration such as inertial focusing (using curved/spiral sections of fluidic channels) or deterministic lateral flow displacement (using arrays of pillars in a microfluidic flow) may also be used.

A multi-chamber cassette 2038 in FIG. 20 allows multiple cell populations to be cultured in parallel in separate growth chambers 2040 on a single cassette, with fluidic flows for seeding, media exchange, washing, harvesting, etc. switched by on-board pinch valves 2042. Such a cassette may be used for isolating multiple candidate populations (such as clonal populations), or for higher-throughput process development or screening operations.

Cassette 2044 shown in FIG. 20 may be designed for intracellular delivery, for example of gene-editing constructs, with input ports for cells and cargo, a mixing section, and a delivery section 2046. Delivery may be performed via electroporation, squeezed flow, turbulent flows formed by obstructions, laser bubble deformation, and/or other means.

A cell sampling or harvesting cassette 2048 in FIG. 20 has a single port that allows fluid to be collected in a tube 2050. A sterile gas filter 2052 allows gas to be displaced during the operation. Various types of containers may be used on board these cassettes, including containers that may be sterile tube welded off or on to the cassette.

Another example of a functional cassette is a droplet-formation cassette 2054 in FIG. 20, with two t-junctions 2056. In such a configuration, when coupled to the source cassette, flows of source liquid (potentially with cells), encapsulating liquid (for example oil), and then finally the carrying liquid (water-based) may be pushed in to form oil-walled droplets encasing the source material.

FIG. 20 shows a few examples of functional cassettes possible in the present implementations. The key advantages of this cassette-based system include modularity, flexibility, and expandability, while maintaining a low initial cost. Moreover, when paired with aseptic coupling systems and methods, the implementations disclosed herein allow parallel processing of multiple batches/patient samples in a flexible, low-cost environment.

### Bubble-Free Controlled Shear Stress

A controlled, low Reynolds number flow of a well-defined viscosity, in a well-defined geometry, such as a tube or a channel, leads to a well-defined shear stress at the surface of the geometry. However, if a large bubble passes through this channel during the flow, in which the bubble may occupy 50% or more of the channel cross-section, the resulting shear stress on the geometry surface may be far higher, leading to a loss of control. Thus, there is a need in the art to determine whether there are bubbles in a fluid channel in a cell culture system.

Systems and methods disclosed herein include methods of confirming that flowing liquid within a tube or channel is bubble-free before applying high flow rates, thereby reducing the risk of shearing away valuable material (e.g., cultured cells). In an example implementation, a pipette tip may be initially filled with liquid and connected to a cell culture cassette via its fluid connectors. When doing so, bubbles may be introduced. Bubbles can also spontaneously form during cell growth or also spontaneously emerge from cassette surfaces. Bubbles should be purged from the fluidic system before applying a large flow rate and hence creating a large shear stress. Purging can largely take place at a low flow rate by taking advantage of the cassette primarily being oriented vertically and by proper design to remove all facets and structures that trap bubbles, such as steps in fluid thickness. During the course of purging, bubbles may be monitored at some point in the tubing by either integrating commonly available low-cost bubble detectors (infrared and ultrasonic are standard), or treating the tubing like a view window. In the latter case, light may be produced and detected from components outside the cassette (e.g., in the fluidic system). The presence of a bubble in the view window would strongly scatter light. Gentle flows may continue back and forth through the fluid system until there are no longer any signals indicative of bubbles. At that point, a sufficiently high flow rate may be applied to the cassette to achieve the desired shear stress. In some implementations, vibrations may also be used to remove bubbles.

### Burst Chambers/Injectors for Small Quantity Reagents

Currently, if small amounts of reagents are needed within a cell culture growth chamber, consideration for tube size and length needs to be considered. Dead volume in the tubing would require an additional quantity of reagents, which can be costly. Additionally, methods to flush the reagents with inert fluids would compromise the concentration of such reagents. Thus, there is a need in the art for safe, efficient methods for introducing small quantities of reagents to a cell culture.

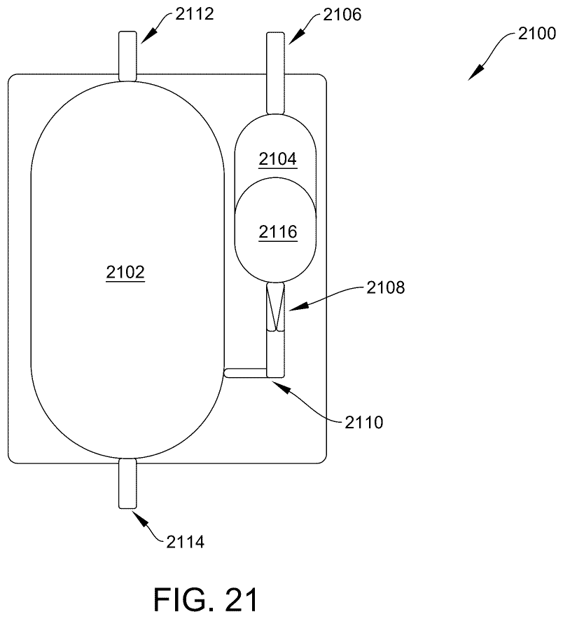

Systems and methods disclosed herein include the use of burst chambers/injectors to introduce small quantities of reagents to a cell culture chamber. FIG. 21 is a diagram illustrating a cell culture container 2100 in accordance with various implementations. The cell culture container 2100 (e.g., a closed cassette) includes a cell culture chamber 2102 and a small reagent chamber 2104. A pneumatic port 2106 and a one-way valve 2108 are connected to the reagent chamber 2104, with a pathway 2110 from the valve 2108 to the cell culture chamber 2102. Fluidic pathways into and out of the cell culture chamber 2102 are established through ports 2112, 2114. Prior to use, the reagent chamber 2104 is partially filled with reagent 2116. During operation, the cell culture chamber 2102 is filled with media and cells. To inject reagent 2116, media is flowed through the cell culture chamber 2102 while the reagent chamber 2104 is pressurized such that the pressure exceeds the burst pressure of the one-way valve 2108. Once the one-way valve 2108 is opened, the reagent 2116 is allowed to enter the cell culture chamber 2102, and the reagent 2116 is mixed into the chamber media. Mixing may be further accomplished via mechanical means such as repeated mechanical depression upon a face of the chamber, or through rotational motion, both of which are described herein.

### Reusable/Recyclable Cassette Designs

Most cell culture containers for biological operations, including cell growth, are single-use and enter the waste stream after the completion of a given experiment. These containers often contain potentially reusable or recyclable components. However, their design prevents the entry of reusable and recyclable materials into the appropriate streams. This results in a growing burden on waste streams and related costs. This is especially burdensome for biohazard waste streams that have higher processing costs. Thus, there is a need in the art for ways to reuse components of cell culture containers.

Systems and methods disclosed herein include a cassette design that permits easy separation of components into the following categories: reusable components, recyclable components, and single-use components that will enter the appropriate waste streams. Material selection for each component category may be made to achieve the category's goal. Inspection processes for component requalification and component lifetimes may be specified for reusable components. Reusable components may include, for example, outer frames, inner frames, screws, steel discs, and pipette adapters. Recyclable components may include, for example, tubing, inner frames, and outer frames. Other components of the cassette may be considered single-use only. A cassette may be broken down into its three component groups with a single tool or no tool. Materials for reusable components are selected to withstand disinfection and sterilization prior to reuse. The cell culture chamber may be removed intact from the inner frame of the cassette to ensure no experimental waste is released during disassembly.

### Commissioning and Parameter Capture During Manufacture of Fluidic Cassettes

A fluidic cassette system for all-optical bioprocess and cell culture processes may have physical, dimensional, and optical characteristics that vary unit-to-unit, which may have impacts on optical or fluidic process steps that occur over the life of the cassette. For example, cassettes may feature optical fiducial marks to allow repeated alignment and co-registration of imagery between measurements and across imaging systems. The absolute positioning of those fiducial marks within the coordinate system defined by the outer envelope of the cassette may vary from unit to unit and thus require exhaustive search by optical systems to find. Other optical characteristics may have variation that requires expanded process operating envelopes for downstream systems, and that may present challenges in designing performant strategies for finding focus or repeatable imagery alignment. These same challenges could also exist in fluidic performance characteristics of the cassette system. Thus, there is a need in the art for improved methods of commissioning and characterization of closed fluidic cassettes for cell culture systems.

Systems and methods disclosed herein include a system for measurement, capture, and cataloging of a cell culture cassette at time of manufacture/assembly, in combination with the optical processing systems for image capture and laser cell manipulation. Measuring a set of critical optical parameters and dimensions during manufacture allows future processing systems to refer to a known reference position and to begin in-process calibration or auto-focus processes at positions close to the previously measured values. In addition, characterizing the actual dimensions and parameters of the cassette unit allows process steps to use optimized strategies rather than accounting for the full range of manufacturing variability between units, and to allow for quality control acceptance or rejection of cassette units.

The parameters to characterize may include, but are not limited to, the precise absolute location of alignment fiducial marks within the coordinate system defined by the outer frame of the fluidic cassette, the shape and size of the alignment fiducial marks and the algorithm with which they should be detected, and the precise optical height of the bottom of the cell culture chamber as assembled into the frame with respect to the coordinate system defined by the outer cassette frame (measured at a regular grid across the entire imaging area of the cell culture chamber).

The frame in which a fluidic cassette is held may include fiducial marks used as reference points for positioning and alignment by an imaging system. The precise position in multiple dimensions (e.g., X, Y axes) of alignment fiducial marks within the fluidic cassette frame may be measured relative to an origin point of the cassette frame's coordinate system. Specifically, the positions may be measured in the as—assembled unit of the cassette, and deviations from the nominal (as—designed) positions may be recorded. The as—assembled positions may then be used for future processing steps rather than the nominal as—designed values. In addition, the precise optical height of the bottom of the fluidic cassettes' cell culture area may be measured and recorded relative to the origin point of the cassette frame's coordinate system. This height may be recorded at a plurality of regular, known positions.

### Robot-Mounted Plug-Connected Fluid Management

Fluidic cassette-based cell processing operations may require a range of mechanical manipulations. Examples of cell culture processing operations include, but are not limited to: seeding cells uniformly, exchanging media efficiently, washing cells or debris, detaching cells from a growth surface, concentrating cells, and harvesting cells. Mechanical actions may include, but not be limited to: tilting, shaking, rotating, and tapping. At least some mechanical actions ideally occur in conjunction with liquid operations. In aseptic or multi-patient processing, this capability has only been solved to date by permanently attaching media and other liquid handling systems to the cell growth chamber, which leads to bulky, complex, and inflexible single-use process cassettes. Thus, there is a need in the art for a more flexible, more scalable mechanical solution for cassette-based systems.

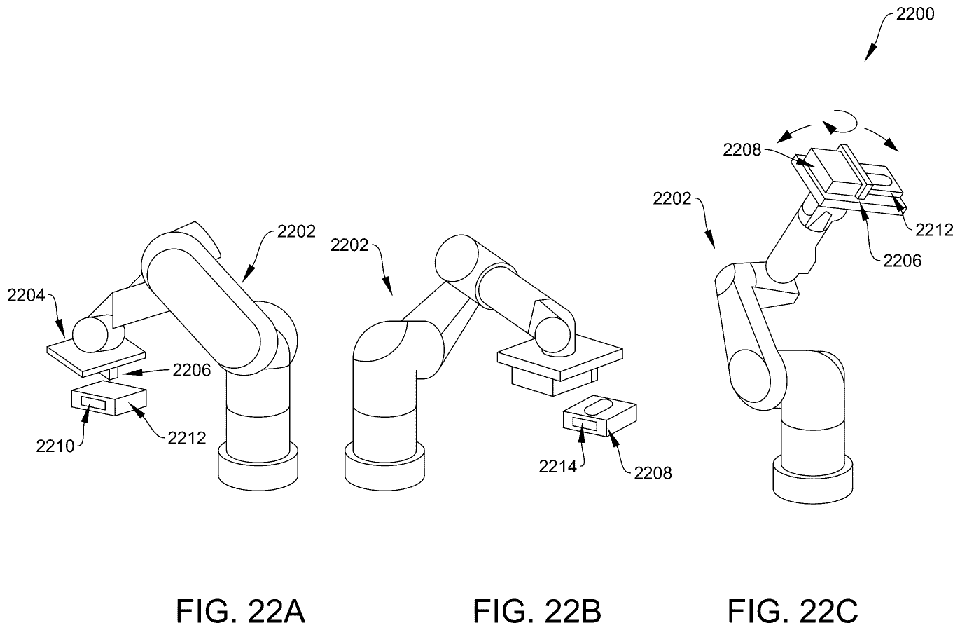

Systems and methods disclosed herein include a pluggable cell process/culture cassette system co-mounted with a fluidic management cartridge and aseptic interconnection system on a movable platform. For example, the system may include an industrial 6-axis robot capable of a wide range of mechanical actions. FIGS. 22A-C are diagrams illustrating a robot-mounted fluid management system 2200 for a cell culture system in accordance with various implementations. The system 2200 in FIG. 22A includes a robotic arm 2202 configured to carry a shared fluidic management system 2204 that interfaces with an aseptic interconnect (or “coupler”) subsystem 2206. The aseptic interconnect subsystem 2206 may be permanent or detachable and used across a plurality of cassettes, dedicated to cassettes from a single batch/patient, or dedicated to a single cassette. The robotic arm 2202 may be configured to retrieve and attach a fluidic supply cartridge 2208 with reusable aseptic connection system 2210.

FIG. 22B shows the robotic arm 2202 retrieving and attaching a cell processing cassette 2212 with a corresponding aseptic connection system 2214. To complete an aseptic connection, the aseptic interconnect system 2206 sterilizes external components of the cartridge 2208 and the cassette 2212. Sterilization may be performed, for example, with vaporized hydrogen peroxide or other sterilant that is supplied via tubing on or in the robotic arm 2202. The aseptic interconnect system 2206 then completes the internal sterile connections between the cartridge 2208 and cassette 2212. FIG. 22C shows the system 2200 during fluidic or other processes in which the robotic arm 2202 may be configured to control the orientation and motion of the assembly (which includes fluidic supply cartridge 2208 and cassette 2212, interconnected by aseptic interconnection subsystem 2206). In some implementations, the robot may set down the assembly for longer-duration processes, such as incubation or coupler/interconnect sterilization dwell times. In some implementations, cables and/or tubes may be connected to the assembly external to the robotic arm 2202, and the arm motion is planned so as to not tangle or compromise these cable/tube assemblies.

The system 2200 is configured to control fluidic/gas exchanges between the supply cartridge 2208 and the cassette 2212, for example by pushing fresh media into the cassette 2212, actuating pinch valves to sample cell/media material, and/or receive spent media.

Simultaneously, the robotic arm 2202 may orient the cassette 2212 in multiple ways, or spin/rock/tap it, as indicated by the arrows in FIG. 22C. In some implementations, the end effector of the robotic arm 2202 may have a rotation stage that allows high angular velocities for centrifugation. In some implementations, the robotic manipulation may be done with the cassette 2212 alone, without a fluidic connection. The robotic arm 2202 may also manipulate the fluidic supply cartridge 2208 to mix or perform other functions prior to connection with the cassette 2212. In some implementations, the aseptic interconnect system 2206 and potentially the entire fluidic management system 2200 may initially not be mounted on the robotic arm 2202. Rather, the robotic arm 2202 or other device may load the fluidic supply cartridge 2208 and cell processing cassette 2212 for aseptic interconnection (which may require some duration for sterilization), and the robotic arm 2202 picks up the ensemble for the fluidic operations, which may be short in duration relative to sterilization.

### Systems and Methods for Controlled-Rate Heating and Degassing

Media and other cell culture fluids are generally stored at 4° C. but they are used in cell culture at ˜37° C. Gas solubility of aqueous fluids generally decreases as temperature is increased, leading to the well-known phenomenon of gas coming out of solution and forming bubbles when cold media is directly injected into fluidic chips. This problem is considered less significant in open culture media (e.g., well plates) because bubbles can generally rise to the surface and pop, though sometimes bubbles do indeed get caught on the sides of wells, leading to cell destruction. However, there is a need in the art to prevent bubble formation in closed cell culture container formats (e.g., closed cassettes).

Systems and methods disclosed herein include methods for conveying media into a cell culture chamber that can be excited by ultrasonic energy while slowly warming to an intermediate temperature between 4 and 37° C. Simultaneously, a vacuum may be applied to reduce the equilibrium amount of dissolved gas. A simple non-cell culture example may be carbonated seltzer water. Supersaturated fluid, being initially in equilibrium at 4° C. with roughly 3 atmospheres of CO₂, may be “degassed” almost instantly by applying ultrasonic energy to it. These bubbles would expand and collapse upon application of vacuum, similar to “degassing polydimethylsiloxane (PDMS),” in which PDMS is a commonly used microfluidic fabrication material. From a media perspective, it would make sense to apply moderate temperatures (e.g., 20° C.) that have little risk of overheating or shocking the proteins, vacuum, and ultrasonic agitation. There seems to be little risk of applying media which is depleted of gas (undersaturated) to cells.

### Systems and Methods to Enable a Plurality of Growth Chambers to Be Imaged and Scanned

Current methods of processing cell culture chambers in optical bioprocesses require complicated automations to move chambers to/from the optical engine. This introduces potential failure points and would require robust solutions to mitigate possible issues. Thus, there is a need in the art for robust methods to move cell culture chambers around an automated cell culture system without disturbing the cell culture.

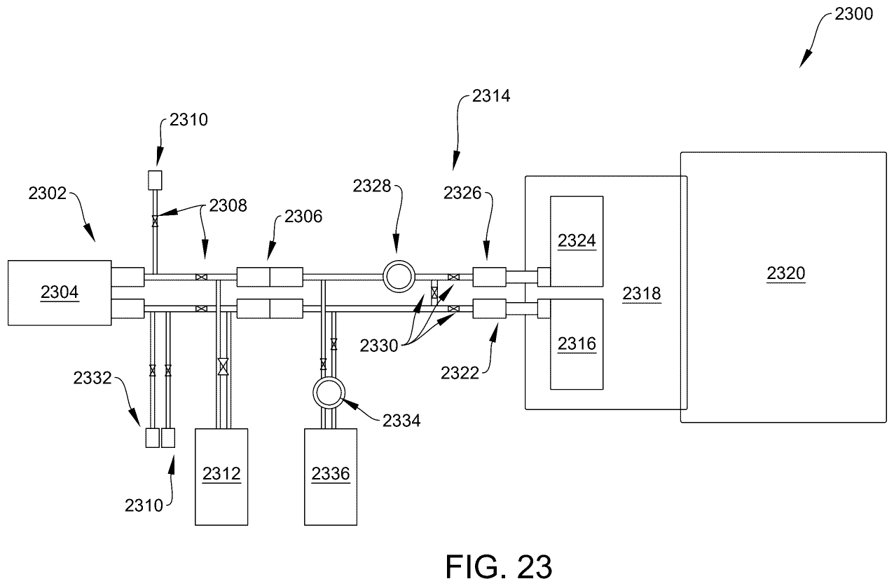

Systems and methods disclosed herein include a cell culture system arranged in a parallel format and an imaging/scanning device configured to sufficiently image and scan a plurality of cell culture chambers. This arrangement enables parallel fluidic operations on all chambers before, during, or after imaging/scanning depending on application. FIG. 23 is a diagram illustrating a cell culture system 2300 in accordance with various implementations. The system 2300 includes at least one single-use cassette 2302 that includes a cell culture chamber 2304 with connectors 2306, a plurality of manually controlled pinch clamps 2308, single-use aseptic connections 2310, and a fluid rinse bag 2312. The single-use cassette 2302 is connected to a tube set 2314 via connectors 2306. Fluid is added to a media container 2316 which is placed into an isolation chamber 2318 that is housed in a biosafety cabinet 2320. The media container 2316 is connected to the tube set 2314 via a connector 2322. Similarly, a waste container 2324, also inside the isolation chamber 2318, is connected to the tube set 2314 via connector 2326. Tube set 2314 includes a peristaltic pump 2328 and electronic pinch valves 2330, which are all normally closed.

During priming, manual pinch valves 2308 between the cell culture chamber 2304 and the connectors 2306 are opened. Pinch valves 2330 leading from the waste container 2324 and the media container 2316 to the tube set 2314 are also opened. Pump 2328 is activated, pulling liquid from the media container 2316 through the cell culture chamber 2304 and ending at the waste container 2324.

During seeding, once the system 2300 is primed, a source of cells is connected to one of the aseptic connections 2310. Manual pinch valves 2308 between the cell culture chamber 2304 and the connectors 2306 are closed, and the valve between the two lines of the tubing set 2314 is opened. Pump 2328 is activated and fluid is circulated through the cell culture chamber 2304. A user may add cells with a syringe via the open aseptic connection 2310. The syringe may be left connected through the remainder of the operation. After some period, pump 2328 stops, and the valve between the lines of the tubing set 2314 is closed.

During cell feeding, pinch valves 2330 leading from the waste container 2324 and the media container 2316 to the tube set 2314 are opened and pump 2328 is activated, delivering fresh media to the cell culture chamber 2304. Alternatively, pinch valves 2330 leading from the waste container 2324 and the media container 2316 may be closed and the valve 2330 between the lines of the tube set 2314 opened, with pump 2328 activated to circulate media within the cell culture chamber 2304.

During cell washing, to remove debris and dead, lifted cells, pinch valves 2330 leading from the waste container 2324 and the media container 2316 to the tube set 2314 are opened and the pump 2328 is activated, delivering fresh media to the cell culture chamber 2304. Spent media will be sent to the waste container 2324. Alternatively, pinch valves 2330 leading from the waste container 2324 and the media container 2316 to the tube set 2314 may be closed and the valve 2330 between the lines of the tube set 2314 opened, with pump 2328 activated to circulate media within the cell culture chamber 2304.

For media and waste container replacement, a user may access the containers 2316, 2326 via a door on the isolation chamber 2318. The user may open the door and remove the waste and media containers 2326, 2316. Users may close the door, where a sterilization cycle may begin. On completion of sterilization, the user opens the door and installs new media and waste containers 2316, 2326. The user then closes the door and the system 2300 can continue its operations.

For cell lifting, a syringe filled with a lifting agent may be connected to connection 2332. The valve 2330 between the lines of the tube set 2314 is opened and pump 2328 is activated to circulate media within the cell culture chamber 2304. A pinch clamp between the connection 2332 and the cell culture chamber 2304 is opened, as well as the valve between the waste container 2326 and the pump 2328. The lifting agent is pulled into connection 2332. Pump 2328 is stopped once the lifting agent is inside the cell culture chamber 2304, and then the pump 2328 is stopped and the valves in the tubing set 2314 are closed.

For removing the cell culture chamber 2304 from the system 2300, a user closes clamps 2308 between the cell culture chamber 2304 and connectors, and opens the clamp leading to the fluid rinse bag 2312. The user then starts pump 2334 and fluid is pulled from the fluid rinse bag 2312 into collection bag 2336. Check valves leading to the pump 2334 from the tubing set 2314 are opened due to the pressure differential caused by the pump 2334. Once fluid is depleted from the fluid rinse bag 2312, pump 2334 is stopped and the clamp leading to the fluid rinse bag 2312 is closed. The user disconnects connectors 2306 from the tube set 2314 and the single-use cassette subsystem 2302 may be removed from the system 2300.

For cell removal, a user brings the subsystem 2302 to a sterile environment (e.g., biosafety cabinet 2320 or other isolator). The user connects an empty syringe to one of the aseptic connections 2310. The user opens the clamps leading from the aseptic connection 2310 to the tube set 2314, and then pulls a syringe and draws liquid from the cell culture chamber 2304 into the syringe. After collection of the liquid, the clamps leading from the aseptic connection 2310 to the tube set 2314 are closed and the user removes the syringe from the subsystem. The syringe is capped and removed from the sterile environment.

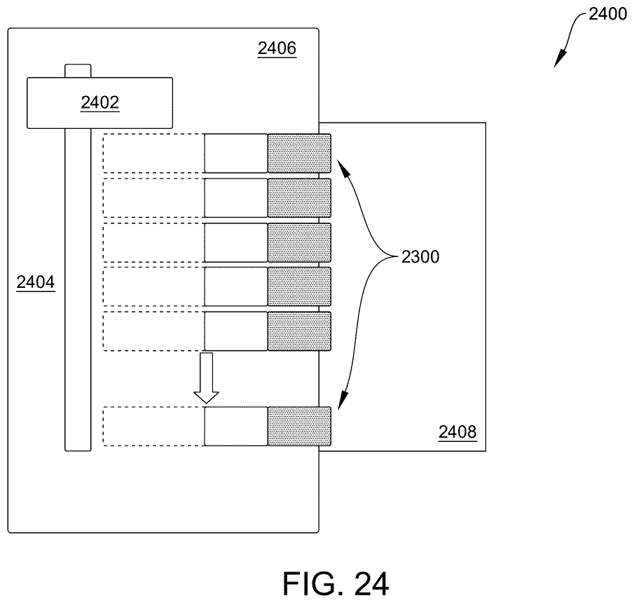

FIG. 24 is a diagram illustrating a modular bioprocessing system 2400 in accordance with various implementations. A plurality of the cell culture systems 2300 may be arranged in the bioprocessing system 2400 such that a movable imaging and scanning system 2402 can sufficiently image and scan individual cell culture chambers in the cell culture systems 2300. The number of closed systems is driven by the number of parallel cell culture chambers that will be bioprocessed with stage 2404 lengthened to accommodate the additional chambers. The closed cell culture systems 2300, optical scanning system 2402, and stage 2404 may be housed in an environmental control chamber 806. Entrance into the isolation chambers of the cell culture systems 2300 may be done through a sterile environment 2408. During imaging and/or scanning operations in system 2400, cell feeding and cell washing operations are also available.

### Remote Cassette/Process Monitoring and Troubleshooting

Over the service life of a laboratory well plate or similar cell culture container assembled from multiple components and assembly processes, aspects of the container can wear to the point of impacting performance or leading to complete experimental failure. This can include the development of unpredictable blockages in flow lines, failure of joints or seams, deformation, stress fractures, or failures of glue bonds. Identifying such issues as they begin to manifest in a container is currently the responsibility of the operator, done by observation during an experiment or post-mortem by tying failed experiment results back to issues with container integrity. Failures in containers are therefore unpredictable and may require manual inspection to be part of experimental standard operating procedures. Thus, there is a need in the art for methods to remotely and dynamically monitor the operational status of cell culture containers in a large-scale cell culture system.

Systems and methods disclosed herein include a monitoring system that includes multiple sensors (e.g., imaging, pressure, and pH) and other methods that will detect emerging defects in cell culture containers throughout the container usage lifetime or experiment. The monitoring system allows for the identification of monitoring points in an associated consumable. Each monitoring point may be assigned to one or more sensors, and, if applicable, warning and failure thresholds. During each run or cycle of the monitoring system, it may be configured to acquire a set of sensor readings for each monitoring point. The resulting readings are presented in a dashboard and changes from previous monitoring cycles are presented. The ongoing changes (or lack thereof) in sensor readings per monitoring point are an indication of wear for that monitoring point. Wear can be displayed with respect to a single monitoring point, groups of monitoring points, or the entire consumable. Wear can be compared to other cell culture containers simultaneously in service or against the history of all.

Methods of wear detection will vary by sensor. Thresholds for wear may be specified for the particular cell culture container and can be adjusted by the operator as needed. Methods of detection include, but are not limited to: (1) the integrity of seals (e.g., adhesive, mechanical, heat-based) and interfaces (e.g., ports, multiple materials) by comparing an image of the relevant component with a template describing an integral component (wear may be described by maintaining a history of such comparisons and showing the variation, or lack thereof, over time); (2) warping or deformation of an element of the cell culture container may be detected by comparing a current image with an ideal image and a set of located points in the ideal image describing corners and edges of the container and the distances between them—the current image is processed to identify the edges and corners and compare them to the ideal set and changes beyond a predefined threshold indicate warping, deformation, or misalignment; (3) blockages may be detected by comparing current images and pressure sensor readings to an ideal set to look for an accumulation of material and reduced flow in a specific area in the cell culture container; (4) cell growth or fouling can be detected by comparing current images of a particular area where fouling or excess growth is a concern with an ideal image, showing either no fouling or an acceptable amount of fouling; (5) ability to hold a pressure setpoint; (6) ability to hold a temperature setpoint; and (7) ability to hold a humidity setpoint.

Each cell culture container may be uniquely identified in a database such that the complete set of monitoring point data, across all experiments, is available for each cell culture container. At any point in time, a report on wear is generated, highlighting locations that have changed from the previous runs and showing wear trends for each location for the current container and against the overall history of all tracked containers. Cell culture containers may be designed in such a way as to lend themselves to monitoring. This can include the use of clear materials to make multiple, stacked-up layers, joints, bond and scaling lines, visible to imaging systems, extra ports for the connection of pressure and temperature sensors, and points where two or more sensor readings may be correlated (e.g., pressure and imaging to show a blockage).

### Magnetically-Actuated Aseptic Connectors

The design and manufacturing of reusable aseptic connectors for bioprocessing is a complex area often involving nested sets of mechanical components. One prior art approach is to have an inner connection in which an aseptic fluidic connection is made, but only after an outer connection that shields this aseptic portion is made. However, this often requires multiple mechanical actuations, and at the same time requires complete isolation of the aseptic area within the connector. The size, cost, and bulk of such connectors can be significant, in part due to the complexity of the mechanical actuators. Additionally, any gap in the mechanical structure may be an entry point for contamination. Thus, there is a need in the art for low-cost aseptic connectors that can easily be used in cell culture systems.

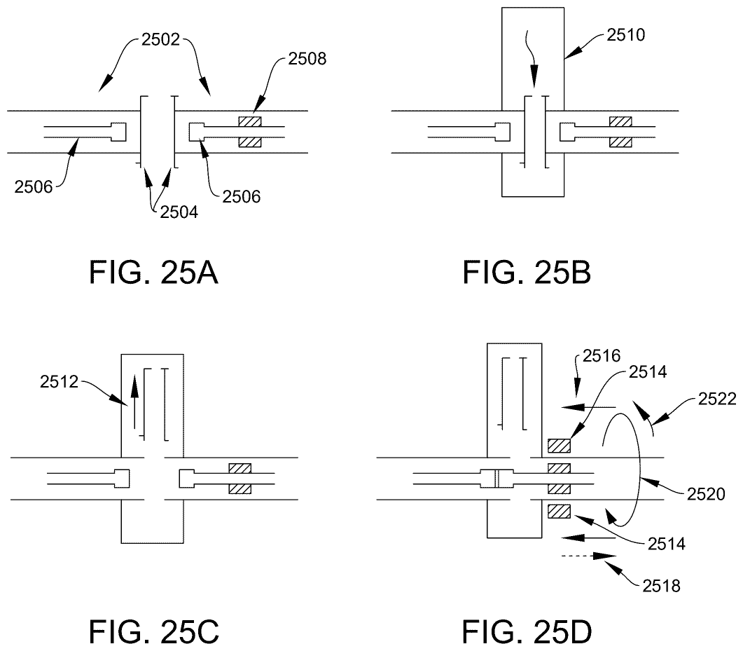

The systems and methods disclosed herein include reusable aseptic connectors, in which one or more mechanical motions is achieved by a magnetic coupling system, enabling translation, rotation, coupling, decoupling, opening, or closing of pinch or other valves within the assembly using external magnetic or electromagnetic components. FIGS. 25A-D are diagrams illustrating a reusable magnetically-actuated aseptic connector in accordance with various implementations. FIGS. 25A-D illustrate a sequence of steps for using the aseptic connector. FIG. 25A shows a two-piece connector system that includes outside protective sleeves 2502 with doors/caps 2504 and is designed to operate in a non-sterile environment while protecting the internal connectors 2506, which are used to transfer liquid aseptically. In some implementations, one of the internal connectors 2506 may have magnetic elements 2508 mounted on it to allow actuation.

FIG. 25B shows the initial step in making a connection, which is using a sealing collar to enclose a space 2510 that is sterilized using a sterilant, UV light, heat, plasma, or other means. FIG. 25C shows the second step in making a connection, which is the removal of the external protecting doors or caps 2504, as indicated by arrow 2512. FIG. 25D shows the final step of making a connection, which is the translation and liquid coupling of the internal connector, by use of external magnetic components 2514 that match the internal magnets 2508. Here, a translation step (as illustrated by arrow 2516) is performed by moving the external magnets. Force feedback (illustrated by arrow 2518) may be used to ascertain whether the target position has been achieved. In other implementations, a rotating motion (illustrated by arrow 2520) may also be used, again with force feedback (illustrated by arrow 2522). These motions and force feedback measurements may be done by hand, but in other cases may be performed by an automated system.

Multiple components within the aseptic connector may be magnetically actuated. For example, the external shield doors or caps may be magnetically translated and/or rotated. In other examples, internal valves may be magnetically actuated externally to open or close fluidic paths. External magnetic fields may be applied and moved using permanent magnets such as rare-earth magnets, or using electromagnets that may turn on/off or control the magnetic fields, and/or translate/rotate the magnetic fields.

### Multi-Channel Aseptic Connectors

Reusable aseptic connections for small bioreactors, for example for single-patient bioprocessing, are often very expensive, bulky, and complex. Additionally, such aseptic connections may not be fit for use in other cell culture container formats. For example, closed cell culture cassettes that support fluidic operations (e.g., fluid, air) are generally compact and have multiple input/output points that require multiple aseptic connectors. Aseptic connectors that are used in large format bioreactors cannot be adapted to small format cassettes. Thus, there is a need in the art for compact aseptic connection mechanisms that support multiple fluid channels.

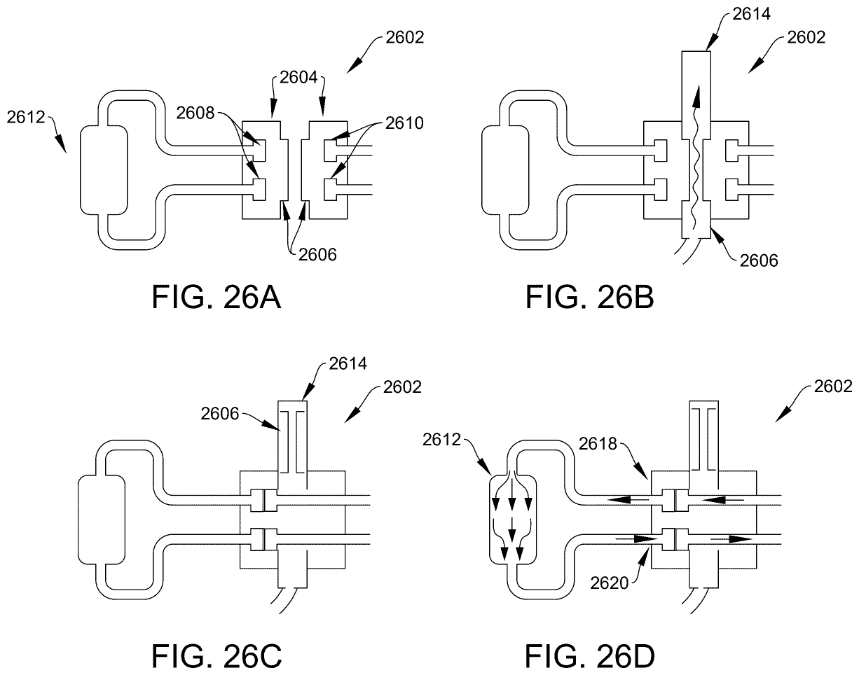

The systems and methods disclosed herein include a reusable aseptic connector that includes a single outer body with doors/caps that is configured to isolate an interior area with multiple sets of interior connectors. Using such a design, a single set of initial (exterior) connections may be made once, the components of the exterior connector that are ultimately exposed to the aseptic components may be sterilized in one pass, the doors/caps moved to open the interior in one motion, and/or the interior connectors may be translated or rotated to couple the aseptic fluid channels together. Thus, the overhead, complexity, cost, bulk, and risk of failure of multiple independent aseptic connections is removed. FIGS. 26A-D illustrate an example implementation of a multi-channel aseptic connector in accordance with various implementations.

FIG. 26A shows an aseptic connector 2602 having outer protective casings 2604 and doors/caps 2606 sealing in a sterile space containing fluidic connector sets 2608, 2610. Fluid connector set 2608 may be connected to bioreactor 2612 while fluid connector set 2610 may be connected to a fluid handling system. FIG. 26B shows both parts of the aseptic connector 2602 brought together and sealed to an interconnection sleeve 2614, which includes a port or ports 2616 for a sterilant. The inside of the interconnection sleeve may be sterilized. After sterilization of the external portions of the aseptic connector 2602, FIG. 26C shows the outer doors/caps 2606 may be retracted into the interconnection sleeve 2614, allowing for internal aseptic fluid connections to be made. After the aseptic fluid connections are made, FIG. 26D shows fluid flowing through the established aseptic connections, with a fluid management system simultaneously pushing in new media 2618 and pulling out spent media 2620, for example, to exchange the media in the bioreactor 2612.

### High-Temperature Sterilization-Enabled Reusable Aseptic Connectors

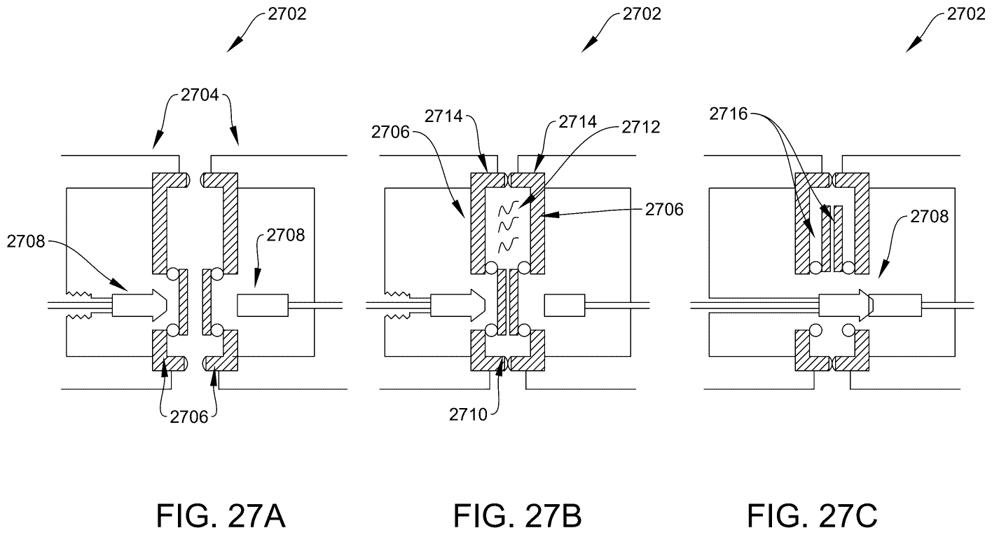

There is a scarcity of reusable aseptic connectors for use in small-batch bioprocessing, such as patient-specific cell manufacturing. There is a need in the art for aseptic connection solutions that prevent contamination of sterile liquids. The systems and methods disclosed herein and shown in FIGS. 27A-C illustrate reusable aseptic connectors that are compatible with high-temperature sterilization in accordance with various implementations. This sterilization may be performed multiple times on the same connector, and may be performed to remove microorganisms prior to fluid connection. Heat sterilization is a proven method to remove contaminants in aseptic bioprocessing systems. Examples of heat treatment include brief dry heating of copper wafer to 300° C. in certain sterile tube welding systems to sterilize its surfaces which come into contact with the interior of aseptic tubing. For dry heat sterilization, there are known time-temperature relationships used to achieve, for example, 6-log (factor of 1 million) reduction in surviving microorganisms. This can range from 1 hour at 170° C. to just seconds at 300° C. based on available time-temperature protocols and rate equations. In present implementations, a short-duration, high-temperature cycle may reduce connection time, as well as maximally localize heating to the front faces of the connectors. Other heat treatments include “moist” or steam and pressure cycles such as those used in autoclaving. These generally require lower peak temperatures for the same effect, but necessitate management of moisture and overpressures. All of these known thermal sterilization treatments may be applied to the present implementations. FIG. 27A illustrates an aseptic connector 2702 having an outside housing 2704 and front faces 2706 which are constructed using heat-resistant materials. For example, the exposed front-facing components 2706 may be constructed from copper or alloys thereof, which have naturally antimicrobial properties, may be exposed to high temperatures for heat sterilization, and have very high thermal conductivity to ensure uniform elevation to the sterilization temperature. The housing 2704 and the front faces 2706 are configured to maintain a sterile enclosure around internal connectors 2708.

FIG. 27B shows the first step of the connection process. The front faces 2706 of the connectors are sealed together mechanically to form a reliable seal 2710 around an interior space 2712. Energy 2714 is then delivered to the front face materials to rapidly heat the front faces 2706 as well as the interior space 2712. The energy may be delivered electrically, and the front face assemblies contain/function as resistive heaters. In other implementations, inductive heating (with an external coil) may be used. In other implementations, hot gas, steam, or plasma may be delivered into the interior space 2712 via an included connector. In some implementations, an external assembly may be thermally connected and provide heat flux. In other implementations, optical heating, such as by a filament or laser, may be used. The purpose of the heating is to sterilize the interior front faces 2706 and interior space 2712 prior to exposing sterile connectors. FIG. 27C shows the final steps of the connection process, in which doors 2716 of the front faces 2706 are moved aside and the interior sterile connectors 2708 are engaged.

Self-Rinsing Aseptic Media Reservoir with Pluggable Interface for Pipette Tip

Some cell culture media, such as iPSC culture media with thermolabile growth factors, should be stored at certain temperatures, such as 4° C. for the aforementioned cell culture media. Before introduction to a cell culture, this cell culture media must be brought to a higher temperature (between 20-37° C.) and also ideally gas equilibrated to 5% CO₂ and 2-21% O₂, depending on the bioprocess. There is a need in the art to handle and prepare these kinds of cell culture media in an automated cell culture system, while ensuring minimal evaporation, no salt precipitation left from dried up media, and repeatable aseptic liquid transfers over a period of time.

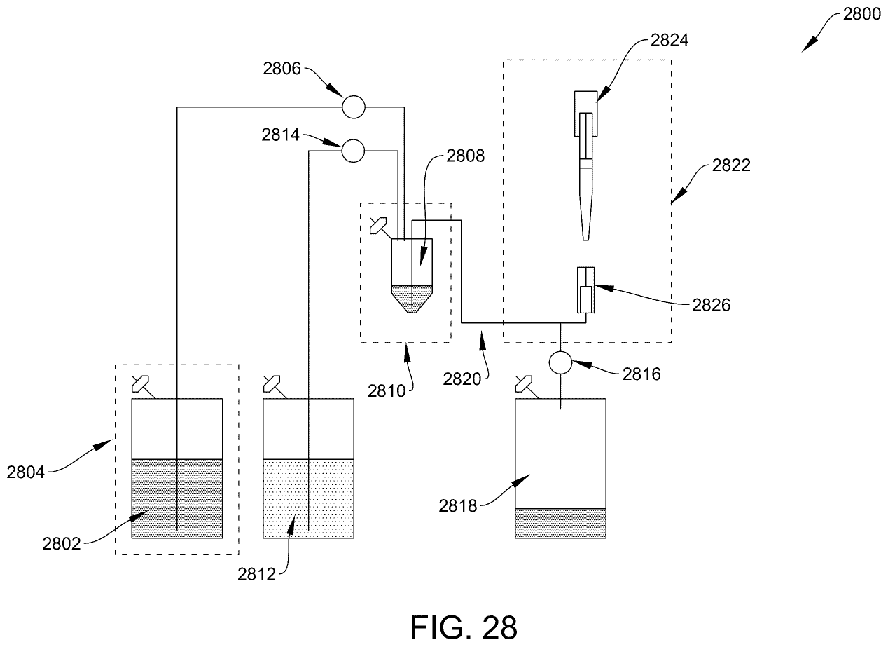

FIG. 28 is a diagram illustrating a fluidic system 2800 for a cell culture system in accordance with various implementations. The fluid handling system 2800 includes a vented, sterile source container 2802 of cell culture media located in a first controlled environment 2804, which ensures that cell culture media stored in the source container 2802 remains at a controlled temperature (e.g., 4° C.). Additionally, the environment may be pressure and/or gas concentration controlled to pre-set gas concentrations in the chilled media to extend the lifetime of the media. For example, since gas solubility is higher at lower temperature, the media may be stored at a net vacuum relative to the ultimate operating environment, to minimize the eventual need for gas concentration equilibration. A low-oxygen environment may further be beneficial to extend shelf life or maintain quality of media. Furthermore, the environment may be shielded from light, in particular short-wavelength light, to prevent light-based degradation of media or reagents. The source container 2802 may include a filtered vent (as shown in FIG. 28), may be a gas-permeable bag without vent, or in some cases a gas-impermeable bag that maintains pre-set gas concentrations. The cell culture media may be drawn from the source container 2802 using the media-line peristaltic pump 2806 into a smaller vented reservoir 2808 held in a second controlled environment 2810 at a desired temperature and gas mix. The smaller vented reservoir 2808 may be used to equilibrate the cell culture media to desired temperatures and gas concentrations before the media is exposed to a cell culture. A vented container 2812 containing deionized water may also feed into the reservoir 2808 through the water-line peristaltic pump 2814. The reservoir 2808 may be drained using a third waste-line peristaltic pump 2816 into a sterile, vented waste container 2818.

A line of tubing 2820 may lead from the reservoir 2808 to a sterile liquid handling enclosure 2822 for running a bioprocess. After priming the tubing 2820 with fresh, equilibrated media from the reservoir, a pipette-based liquid handler 2824 may be configured to withdraw media through a self-sealing pluggable interface 2826. After aspirating media for a bioprocess operation, the reservoir 2808 may be drained and rinsed by sequentially running the waste-line peristaltic pump 2816 into the waste container 2818 and the water-line peristaltic pump 2814 from the deionized water container 2812 for a desired number of cycles. The reservoir 2808 is left dry at the end of these drain-rinse cycles following one final drain operation using the waste-line peristaltic pump 2816. Final rinsing of the pluggable interface 2826 may be performed by pulling deionized water through the pluggable interface 2826 with another clean pipette tip. The fluidic system 2800 may be fully sealed and may therefore be assembled and sterilized according to standard single-use bioprocessing tubing set assembly techniques. Furthermore, it may be operated in a non-sterile environment, with the exception of the pluggable interface 2826 which is inside of an enclosure and only punctured by sterile pipette tips.

### Lidless and Lidded Aseptic Connector

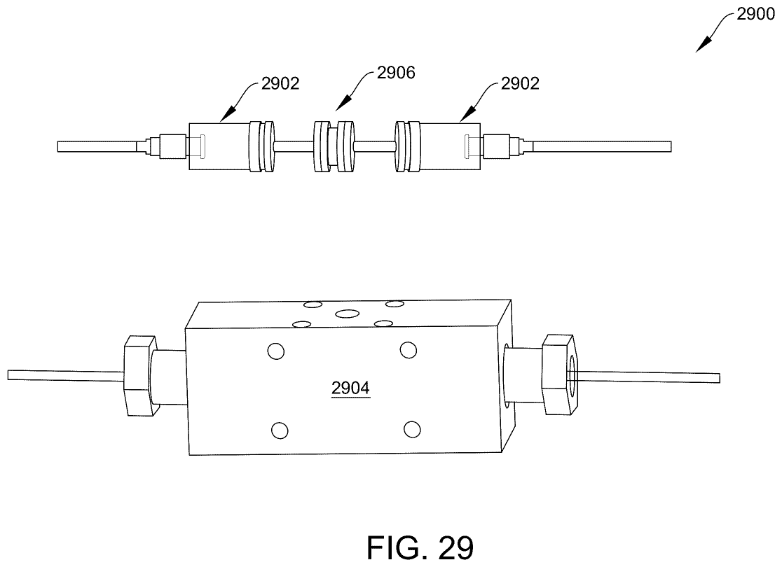

Needleless ports are inexpensive and provide a normally closed valve that may be sterilized with commonly available methods such as alcohol solutions and volatilized hydrogen peroxide. It would be advantageous to be able to convey fluid from one needleless connector to another without violating the sterility of the fluid in either connector. Thus there is a need in the art to adapt cheap, simple connectors into an aseptic environment. The systems and methods disclosed herein include adapting needleless ports such that they provide an aseptic connection. FIG. 29 illustrates a diagram of an aseptic connector 2900 in accordance with various implementations. Two needleless ports 2902 may each be placed into a hermetically sealed block 2904, sealed from the outside and sterilized with a sterilant. Within the sealed block 2904, a sterilized tube 2906 may be used to make a hydraulic, aseptic connection between the ports 2902. In some implementations, the needleless ports 2902 may be augmented with a door that prevents contamination from landing on the ports 2902 while outside of the block 2904, providing a further level of protection. Several designs with outer protective doors or faces are described herein.

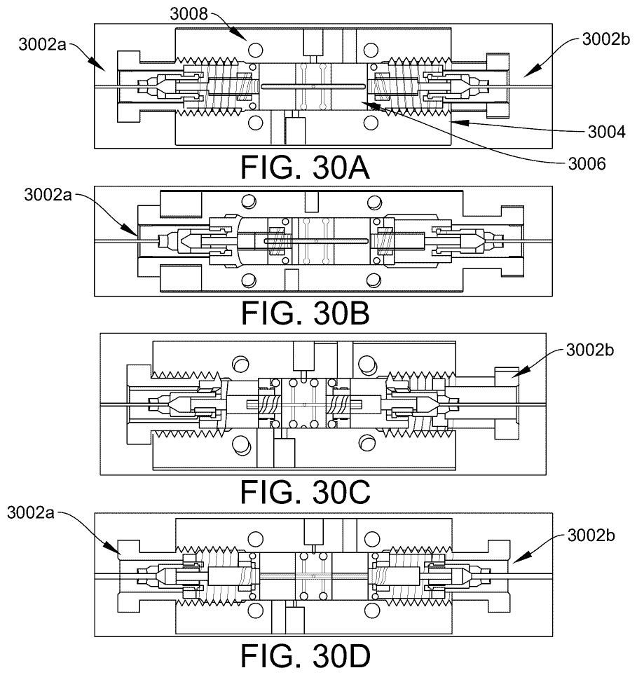

FIGS. 30A-D are diagrams illustrating a method of inserting connectors into a sealed block in accordance with various implementations. FIG. 30A shows two connectors 3002a, 3002b, which may not be sterile, screwed inside block 3004. Inside each connector 3002 may be clean fluid. The connectors 3002a, b should be hydraulically joined to, for example, exchange cell culture media or other standard cell culture unit operations. However, contaminants should not enter into the fluid. When screwed into the block 3004, the connectors 3002a, b are hermetically sealed from the outside. The block 3004 may be cleaned using vacuum suction (through one or more ports) or washed with water, or other methods. A sterilant gas or fluid is passed into the block 3004 through sterilant ports 3008. The sterilant sterilizes both the connectors 3002a, b and an interior 3006 of the block 3004.

FIG. 30B shows further screwing of the connector 3002a so that it penetrates a split-septum within the block 3004. FIG. 30C shows further screwing of the connector 3002b, penetrating its respective split-septum within the block 3004. There is now fluidic communication between the connectors 3002a, b and a peristaltic pump or other device may be used to pump fluid from one to another. FIG. 30D shows the connectors 3002a, b retracted from within the block 3004 and thus fluidically disconnected from each other. A sterilant or flushing liquid may be passed through the block 3004 to remove cell culture media. As a final step (not shown), the connectors 3002a, b may be completely removed from the block 3004.

### Aseptic Connection System Employing Single-Use Seals and Coupling

Reconnectable aseptic connectors are bulky and costly. For ensuring sterility, these aseptic connectors rely on strategies for sterilization of external-facing components before opening to expose interior sterile connections. Such sterilization may be damaging to many components, or dramatically restrict materials choices, particularly if a connection must survive many cycles. Thus, there is a need in the art for reliable, reusable aseptic connectors.

Systems and methods disclosed herein include a reusable coupling component that couples two connectors. The steps for using the contemplated reusable coupling component include (1) mating and then aseptic-compatible treatment of external faces (including sterilization procedures), (2) removal of the external faces into a disposal compartment of the coupler, (3) making and using the internal sterile connection, and (4) deployment of new external faces from a sterile-sealed compartment of the coupler and attachment to the connectors.

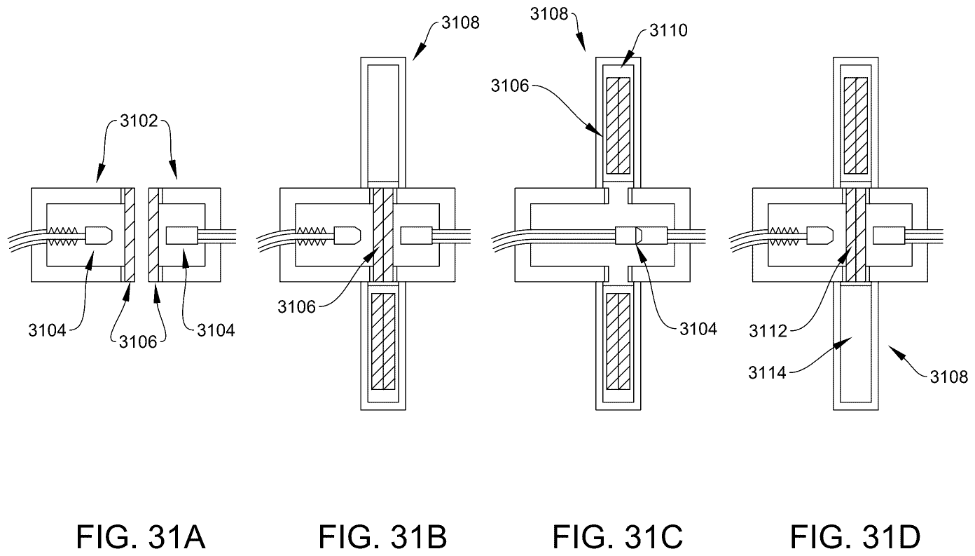

FIGS. 31A-D are diagrams illustrating operation of a reusable aseptic connection system in accordance with various implementations. FIG. 31A shows two aseptic connectors, each with outside housings 3102 containing internal sterile connectors 3104, and having removable outside faces 3106. The faces 3106 may have various implementations, but functionally the faces 3106 should provide a good seal to prevent contamination of the interior of the connector, and may be sterilizable externally. FIG. 31B shows a connection process using a coupling component 3108, which establishes a sealed volume around the two connectors and front faces 3106. The sealed interior may then be sterilized using various means, including but not limited to use of a sterilant, heat, ultraviolet radiation, or plasma. The present implementations allow the front faces 3106 to be destructively processed to ensure sterility in the interior of the connector. For example, the front faces 3106 may be thermally processed in a manner that melts them into one another, scaling any potential contaminants into the material.

FIG. 31C shows the coupled aseptic connection after sterile processing. The front faces 3106 may be transferred to a disposal compartment 3110 in the coupling component 3108. The interior of the coupling component 3108 may now allow an internal aseptic connection between the sterile connectors 3104. FIG. 31D shows the resealing of the aseptic connectors after transactions are completed using the internal sterile connectors 3104. New front faces 3112 may be retrieved from a sterile-sealed storage compartment 3114 in the coupling component 3108, and placed on the front of the sterile connectors 3104, where they reseal the sterile connectors 3104 from the outside environment. In some implementations, sealing may be done mechanically against gaskets. In other implementations, sealing may also be done thermally, in which the new front faces 3112 are heated and adhered to the external surface of the sterile connectors 3104. After sealing, the coupling component 3108 may be removed from the aseptic connectors and sent to disposal or recycling (in cases in which mechanical components are sterilized, the compartments may be re-sealed with a fresh set of external front faces).

Consumables with Aseptic Connectors Including Inner and Outer Pieces

A sealed fluidic cassette with reusable, pluggable aseptic connectors for media changes and other fluidic/gas operations would enable scalable and multi-patient cell manufacturing systems. However, an efficient, compact means of fabricating and using such connections is not apparent in the industry. Thus, there is a need in the art to develop aseptic connectors compatible with closed fluidic cell culture containers.

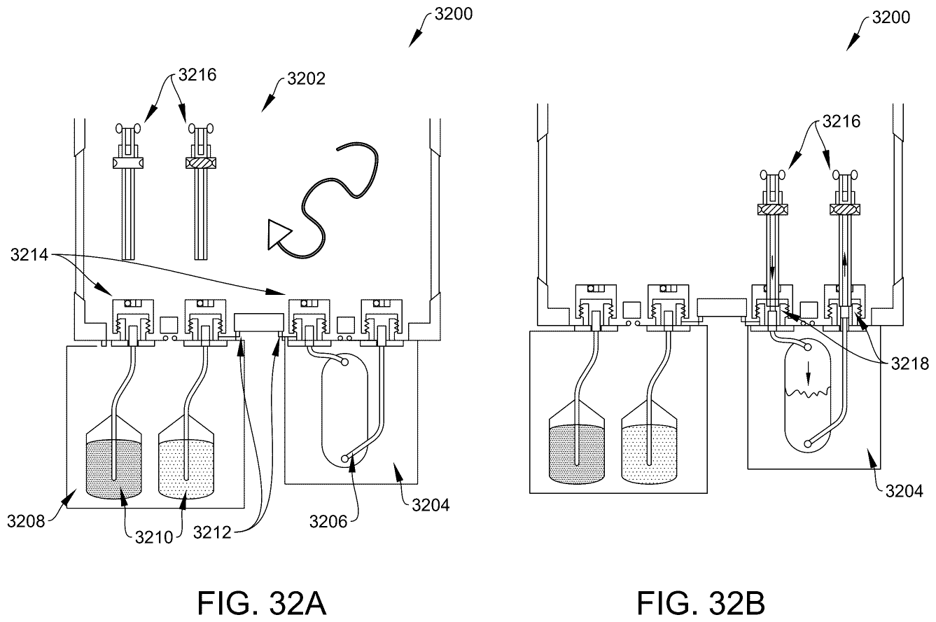

Systems and methods disclosed herein include an aseptic connector that is a combination of an outer removable connector and an inner pluggable connector, and a system for making fluidic transfers from liquid sources/sinks to and from the cell culture/processing cassettes. FIGS. 32A-B are diagrams illustrating an aseptic connector system 3200 in accordance with various implementations. The system 3200 uses two-layer connectors together with a fluidic transfer system to enable flexible liquid handling for aseptic cassettes. FIG. 32A shows an aseptic transfer space 3202 connected to a cell culture cassette 3204 having a cell culture chamber 3206. The aseptic transfer space 3202 is also connected to a fluidic supply cartridge 3208 with reservoirs 3210. The cassette 3204 and cartridge 3208 are attached to the aseptic transfer space 3202 using gaskets 3212. In alternate implementations, individual tubes or flasks may be used in place of a fluidic supply cartridge 3208. In some implementations, an equilibration system such as the one described herein may be employed. In the example shown in FIG. 32A, the fluidic supply cartridge 3208 and cassette 3204 both feature outer protective caps 3214 and inner pluggable fluidic connectors (such as split-septum, self-sealing connectors) which are exposed only to a sterile and/or patient-specific environment.

Once consumables (the cartridge 3208 and the cassette 3204) are sealed to the aseptic transfer space 3202, a sterilization operation is performed. The sterilization process may use a gas sterilant or a liquid sterilant, or other means such as heat or radiation exposure. The sterilization removes any potential contaminants from the interior of the aseptic transfer space 3202, and from the chamber-facing portions of the fluidic supply cartridge 3208 and cell cassette 3204. The outer caps 3214 of these consumables are compatible with sterilization. Examples of such outer caps 3214 are screw caps with automation-compatible mechanical interfaces that are used on sample tubes. In this implementation, automation-compatible screwable caps 3214 are shown in FIG. 32A. Fluidic transfer components 3216 may be sterilized in the aseptic transfer space 3202, or may be supplied from an aseptic dispenser. In the example shown in FIG. 32A, the fluidic transfer components 3216 are shown as pipette tips with air filters.

FIG. 32B shows a fluidic transfer underway in the example implementation of FIG. 32A. After sterilization, the outer caps 3214 may be removed. Typically, the outer caps 3214 are removed only for the duration of a liquid operation (e.g., retrieving liquid from a port, dispensing liquid into a port). In this case, the two liquid transfer components 3216 have been plugged into inner pluggable ports 3218 of the cell culture cassette 3204, which are normally closed, but opened for this operation. Fresh media previously retrieved from the liquid supply cartridge 3208 is then pushed into the cassette 3204 through one of the liquid transfer components 3216, where it displaces used media. The used media is aspirated out by the other liquid transfer component 3216. The liquid exchange process is controlled by pressure/volume controllers attached to the liquid transfer components (not shown).

Following this operation, the liquid transfer components 3216 are withdrawn, the outer caps 3214 are replaced onto the cassette 3204, one cap 3214 covering the waste reservoir pluggable connector on the supply cartridge 3208 is removed, the waste media is dispensed into the supply cartridge 3208, and then the cap 3214 is replaced. At this point, the liquid transfer components 3216 may be disposed of (if they are one-time-use). Another sterilization cycle may be performed to ensure the decontamination of the external connector/cartridge/cassette faces, and then the cassette 3204 and/or supply cartridge 3208 disconnected. In some implementations, the supply cartridge 3208 may be retained to service multiple cell cassettes (for example, when cassettes belonging to a single batch/patient are being processed consecutively). In some implementations, the liquid transfer components 3216 may be used to connect the liquid supply cartridge 3208 directly to the cell cassette 3204, rather than performing two-step transfers.

Cassette Fluidic Handler with Aseptic Loading Features

In a multi-cassette cell culture system, it is often desirable to move cells and liquids in and out of the multiple cassettes, while retaining flexibility in liquid volumes, transactions, and other operations. However, any such operation imparts a risk of contamination, or cross-contamination between cassettes. Thus there is a need in the art for aseptic connections in multi-cassette cell culture systems in which various components may be plugged, unplugged, and re-plugged into various other components.

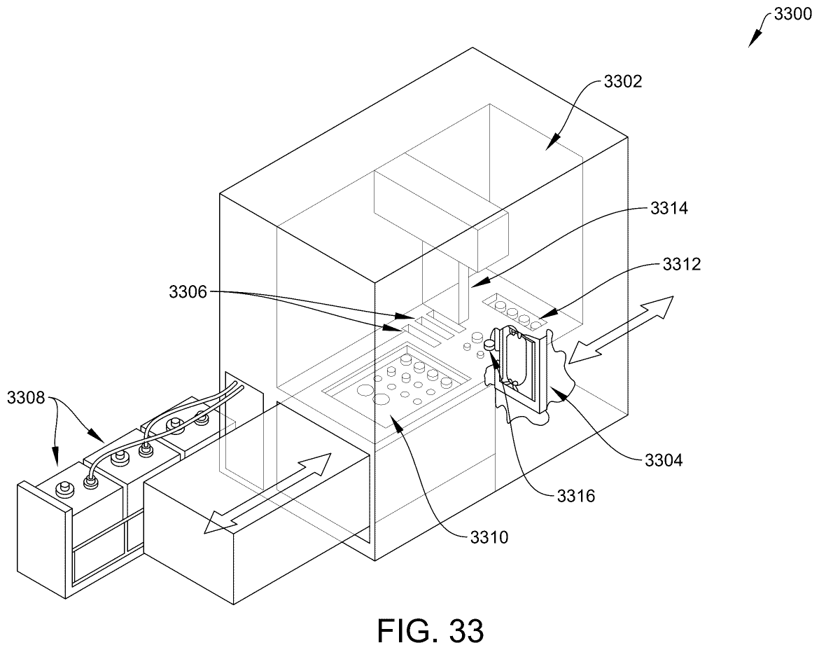

Systems and methods disclosed herein include a fluidic management system with sterilizing airlocks that enables flexible liquid handling for complex bioprocesses and/or a range of bioprocesses, while maintaining a batch-isolated environment and aseptic connections for cell cassettes, media, cell products, and reagents. FIG. 33 is a diagram illustrating a fluidic management system 3300 for use in a cell culture system in accordance with various implementations. The fluidic management system 3300 includes an interior sterile fluid handling space 3302 which serves to transfer liquids between attached fluidic cell cassettes 3304, bulk liquid supply/exhaust features 3306 (which may be open as shown here, or include pluggable liquid ports) that are connected to aseptically-attached containers 3308, and consumables in aseptic tubes/cartridges on tray 3310.

An example operation of the system 3300 is as follows: the cell cassette 3304 may be transported into the system 3300 from another environment. That external environment may include, for example, shared infrastructure for incubation and imaging serving multiple batches of cassettes which may include multiple patient samples. The cassette 3304 includes a face with its aseptic connector portion that is sealed against sterilization airlock 3312. The airlock 3312 is initially covered and separated from the interior liquid handling space 3302. The airlock 3312 is sterilized, including the external connectors of the cassette 3304, for example by filling the airlock 3312 with a sterilant, and then flushing this sterilant out. After sterilization, the airlock 3312 is opened to the interior. Similarly, the supply tray 3310 may be loaded from another environment as well. In some implementations, the cassette 3304 and supply environments may be merged, potentially with a single set of automation transporting cassettes and supply cartridges or trays. Similar to the cassette 3304, the supply tray 3310 is interfaced to a sterilization airlock (initially separated from fluid handling space 3302), where the supplies are sterilized with a sterilant or other sterilization technique, then exposed to the shared fluid handling space 3302. The fluid handling space 3302 may also be sterilized between batches. In this manner, multiple cassettes may be managed, and cross-contamination between different batches/patients may be avoided.

The present implementations may handle multiple batches (or patients) simultaneously or in parallel, with complete sterilization of the shared space between batches, or be dedicated to a single batch for the duration of a run (in which the shared environment may not require re-sterilization during the run). Once attached, an automated capper/de-capper mounted on a robotic gantry head 3314 may de-cap ports on the cassette 3304, and tubes or cartridges in the supply tray 3310, to enable liquid transfer operations. The tray 3310 may hold tubes that are sterile and sealed, but empty, for the purpose of retrieving samples from the cassettes 3304, or for temporary liquid operations such as mixing and dilution. The supply tray 3310 may hold, for example, tubes or cartridges for source cells, ECM, cell media, reagents, washing media, cryopreservation agents, media samples from cassettes, cell samples from cassettes, finished cells from cassettes, and other applications. There may be a temporary holding area 3316 for caps from various consumables. Caps may be held from the top, external-facing side for this purpose, which may be on the robotic gantry head 3314.

In some implementations, all liquids or cells moving in and out of the system 3300 may be transferred through the tray mechanism. In other implementations, in which bulk media volumes are significant, it may be preferable to have a bulk media/waste/wash fluid feed as shown in FIG. 33. Containers 3308 may include a bulk fresh media container and a waste container which may be located in a drawer accessible from the outside environment. This drawer may be maintained at 4° C. If needed, the containers 3308 may be attached and/or detached via aseptic connection means such as tube welding or one-time aseptic connectors. The fresh media may be transferred to one of the supply features 3306, and one of the exhaust features 3306 may allow waste media to be transferred to the waste container. Other holders may be supplied by washing media, for example. The robotic gantry head 3314 may be configured to perform capping, de-capping, and liquid aspiration and dispense operations, including simultaneous aspiration and dispense in cell cassettes.

### Aseptic Coupler Systems and Methods for Bioprocessing Cassettes

Reusable aseptic connectors can have a breakthrough impact on flexible, scalable bioprocessing systems, particularly for small cell batches (including but not limited to patient-specific batches). However, even if the challenges of building a reusable aseptic connector are solved as described herein, several issues remain. For example, if sterilization is used as part of the connection (and potentially disconnection) process, the sterilization may have durations of 15 minutes or more. Further, some operations requiring the fluidic connection may also be lengthy in duration. For example, some media operations must be performed with very low flow. In other cases, a filtration process may require many passes through the filtration system. In extreme cases, perfusion cell culture may require ongoing media flows, with intermittent changes of a media/waste cassette. Thus there is a need in the art for quicker and more efficient methods of establishing aseptic connections in a cell culture system.

Systems and methods disclosed herein include an aseptic coupler that connects together two (or more) cassettes with reusable aseptic connectors, enables sterilization of external components prior to making sterile media connections, and is able to travel while connecting the cassettes to one or more process stations. The coupler may be actuated by a coupling and/or sterilization station. The coupling/decoupling, sterilant injection, sterilant action, sterilant removal, and cell culture process stations may all be different, and available in parallel in different numbers, to allow efficient cassette processing. The coupler may remain engaged to the cassettes briefly for a single liquid/gas operation at a single station, or it may stay engaged with the cassettes for multiple operations at a single or different stations, or it may stay engaged for 23 hours or more, in some cases 5 days or more, with continuous or periodic liquid operations performed.

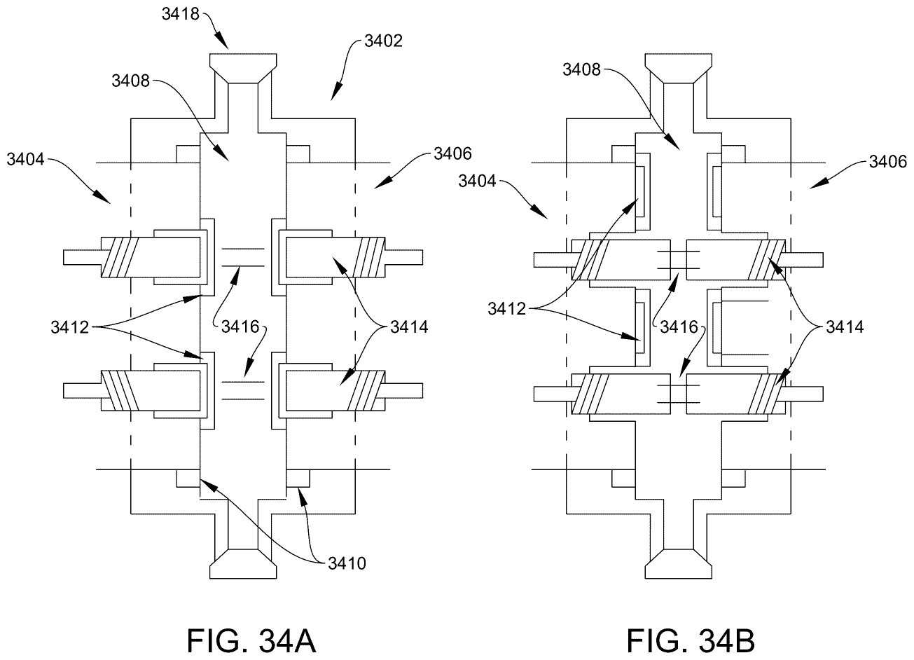

FIGS. 34A-B are diagrams illustrating an aseptic coupler for use in a cell culture system in accordance with various implementations. The aseptic coupler may be configured to couple together two closed cassettes. FIG. 34A shows an aseptic coupler 3402 between cassettes 3404 and 3406. A sealed interior space 3408 has been established between the two cassettes 3404, 3406 via seals 3410 that seal the front faces of the cassettes 3404, 3406 to the interior (“aseptic”) space 3408. In the example shown in FIG. 34A, each cassette 3404, 3406 has two ports, and each port has an outer shield 3412 and an inner fluidic connector 3414. The inner connectors 3414 may be a septum that can be opened via a tube, a diaphragm that can be pierced by a syringe, a self-sealing connector such as a spring-loaded metal connector, or a tube with a pinch valve, as well as other mechanisms. In some implementations, the inner connectors 3414 are protected by the outer shields 3412, which prevent any exchange of materials between the general operating environment and the inner connector face.

The aseptic coupler 3402 includes stationary interconnection tubes 3416 configured to connect fluidic lines on the two cassettes 3404, 3406, and which are sterilized as part of the sterilization procedure. Once the cassettes 3404, 3406 are sealed to the inside space 3408 of the aseptic coupler 3402, sterilization may commence of the interior connectors 3416 and front-facing elements of the cassettes 3404, 3406, including outer protective shields, caps, or doors 3412. The sterilization may be done via a port 3418, and a complementary exit port. For example, the interior space 3408 may be filled with a liquid or gas sterilant including but not limited to: vaporized hydrogen peroxide, ozone, ethylene oxide, peracetic acid, chlorine dioxide, isopropyl alcohol, or sodium hypochlorite solutions. The interior space 3408 may also be filled with a plasma generated by an external source, or on the interior via an external RF source. Alternatively, the interior space 3408 may be heated, electrically or through pressurized steam.

FIG. 34B shows the established aseptic fluidic connections of the cassette/coupler system after sterilization is complete. After sterilization of the interior space 3408, including the front-facing portions of the cassettes 3404, 3406, the outer shields 3412 are removed from the fluidic inner connections 3414. Note this may be done out-of-plane as well to preserve minimum pitch between fluid connections in an array. The outer shields 3412 may be one per connection as shown in FIGS. 34A-B, or in other implementations there may be a single outer shield for multiple connectors. Once the outer shields 3412 are removed, the inner fluid connectors 3414 are exposed and translated towards one another and onto the interconnection tubes 3416 to form contiguous fluidic connections.

During this process, and during use of the cassettes 3404, 3406 in coupled configuration, ports or electrical connections used for sterilization may be disconnected, so the assembly of cassettes 3404, 3406 and aseptic coupler 3402 may be moved freely throughout a workspace, or manipulated mechanically in a variety of ways. Such movements may include, for example, counterflow centrifugation in which the assembly is rotated and fluid is pushed through the coupler simultaneously, mixing processes in which rapid rotations may be used to mix fluids within a fluidic chamber, and mechanical vibration or tapping steps in conjunction with washing to harvest cells. The aseptic coupler 3402 may be configured to maintain a mechanical grip on the cassettes 3404, 3406 and provide good seal integrity around the interior aseptic space 3408. The interior space 3408 may be filled with a gas that helps maintain sterility. It may also be pressurized to minimize the possibility of any contamination from the external environment. The aseptic coupler 3402 may further include sensors that monitor the state of the interior space 3408 (such as pressure relative to the external environment, temperature, flows through the fluidic connections, etc.).

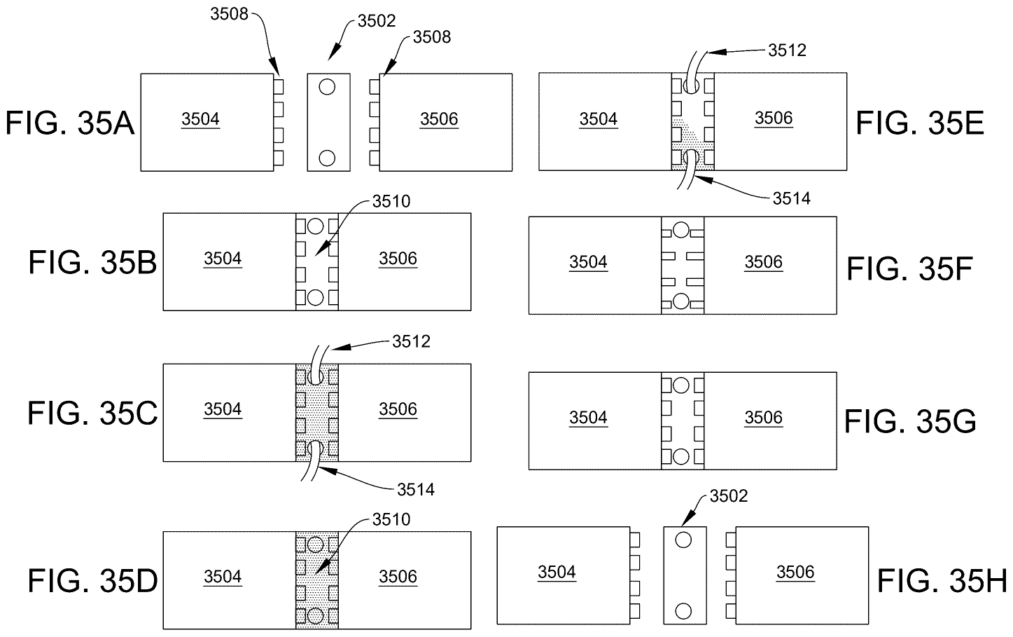

FIGS. 35A-H are diagrams illustrating a method for establishing an aseptic connection using an aseptic connector in accordance with various implementations. FIG. 35A shows an aseptic coupler 3502 (which may be similar to aseptic coupler 3402 in FIGS. 34A-B) used to connect cassettes 3504, 3506 using aseptic fluidic connections 3508 that are sterilized and opened within the aseptic coupler 3502. The cassettes 3504, 3506 may be cell culture cassettes, cell or fluid processing cassettes, fluid supply cassettes, or other cassette types. The present implementations enable interconnection between a wide range of cassettes through a standard interface capable of multiple parallel fluidic or gas connections.

FIG. 35B shows the assembly (the two cassettes 3504, 3506 and the aseptic coupler 3502) mechanically locked together with an interior space 3510 sealed. The connection and locking of cassettes 3504, 3506 to the aseptic coupler 3502 may be done using a range of mechanisms, with cassettes 3504, 3506 attached simultaneously or serially to the aseptic coupler 3502. When not attached to the aseptic coupler 3502, the individual cassettes 3504, 3506 may be aseptically sealed and therefore may be handled, manipulated, imaged, laser-scanned, transported, etc. in a non-sterile space. The cassettes 3504, 3506 may represent multiple batches, including samples from/for multiple patients in the case of autologous or patient-matched processes. In some implementations, the cassettes 3504, 3506 themselves may incorporate the elements of the aseptic coupler 3502, such that cassettes may be joined together directly and the functionality herein is entirely contained in the cassettes, rather than using an intervening coupler.

FIG. 35C shows the beginning of a sterilization process, with connections 3512, 3514 made to the aseptic coupler 3502, for the purpose of introducing sterilant into the interior space 3510. In some implementations, the external connections 3512, 3514 remain throughout the sterilization process (for example, if hot steam or a plasma is used that must be continuously generated by an external piece of equipment). One or more stations in a system may be dedicated to sterilization, which may be the same or different from the stations that perform the mechanical coupling. In other implementations, the space between cassettes may be sterilized using means other than a chemical sterilant, for example by heating or generation of plasma, which may not require external connections.

FIG. 35D shows a case where a sterilant, once introduced, must act for some period of time, and the assembly may be stored for this period disconnected from the sterilization equipment while the sterilant acts on the surfaces of the interior space 3510. All stations in a system employing the present implementations may be configured to provide temperature or other environmental controls. For example, in some cases one portion or the entirety of one of the cassettes may be maintained at 4° C. while the other cassette is maintained at 37° C. FIG. 35E shows sterilant being removed from the interior space 3510 of the aseptic coupler 1002 through one of the external connections 3512, 3514 prior to interconnection of the cassettes.

FIG. 35F shows the interconnection between cassettes 3504, 3506 established in the sterile interior space 3510. The assembly may now be used in a range of operations on a range of pieces of equipment made to perform those operations. This may be as simple as a media exchange (where one cassette is a supply cassette, and the other is a cell culture cassette), but may include more sophisticated or complex operations where liquid and/or gas flows are required between cassettes, for example filtration, centrifugation, mixing, cell harvesting, intracellular delivery, cell sorting, etc. The interconnected assembly may be active only a small fraction of the process time (e.g., for a daily media change), or may be active for the majority of the process time (e.g., continuous perfusion cell culture, with the media/waste cassette changed out only once every 5-15 days).

FIG. 35G shows the disconnection and replacement of outer shields for the cassette connectors as indicated, prior to disassembly. Finally, FIG. 35H shows the disassembly of the cassettes 3504, 3506 from the aseptic coupler 3502. In some implementations, only one cassette may be removed and is replaced with a new cassette, while the other cassette remains locked to the aseptic coupler 3502, such as is the case with the perfusion cell culture described herein, or in a process where a sample passes through a series of functional cassettes, traveling from one to another, back and forth through the aseptic coupler 3502.

The aseptic couplers described herein may be multi-use or one-time-use. In some implementations, one-time-use aseptic couplers may provide fresh external shielding material or components for the attached cassettes upon disconnection. In other implementations, this shielding may be attached to the front faces of the cassettes during connection, in such a manner as to completely cover potentially contaminated faces. In other implementations, sterilant and/or flushing agents may be stored on board the aseptic coupler.

Pluggable Connectors with Bubble Removal

Pluggable, self-sealing connectors are of great benefit to flexible bioprocessing systems, particularly those based on fluidic growth chambers and media/reagent/waste cassettes. However, particularly in all-fluidic systems where cells are maintained or grown in fluidic chambers/channels, bubbles introduced into the system may be detrimental to cell health and overall fluidic system operation. Many pluggable connector designs result in small amounts of trapped gas in the fluidic lines. Thus, there is a need in the art for solutions for trapping or removing bubbles in closed bioprocessing systems.

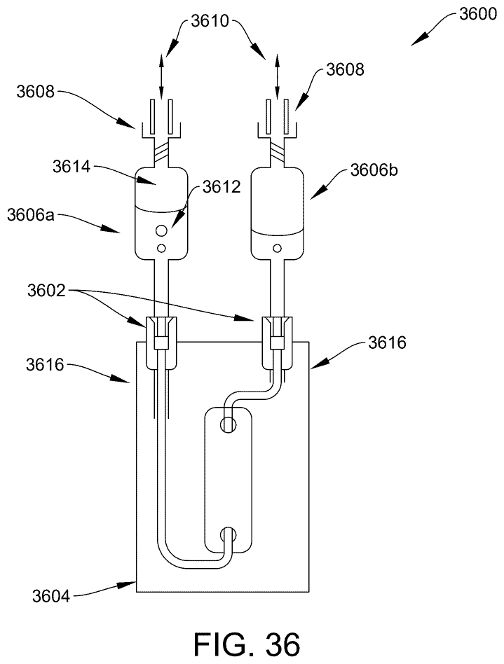

Systems and methods disclosed herein include solutions for trapping and removing bubbles from closed, pluggable fluidic systems. FIG. 36 is a diagram illustrating a fluidic connection system 3600 in accordance with various implementations. The system 3600 may be configured to enable pluggable fluidic connections to fluidic cassettes and cartridges, in which bubbles trapped in the connection process are removed via gravity and other means. The system 3600 includes self-sealing pluggable ports 3602 that are part of a fluidic cassette 3604 and are engaged by fluidic volumes 3606a-b, which are similar to disposable pipette tips but include additional volume for bubble capture and isolation. These volumes 3606a-b are plugged into a gas displacement/pressure control system via connectors 3608 (shown in FIG. 36 with volumes that include gas filters). Upon forming a connection by penetrating the self-sealing connectors of the cassette 3604, pressure/volume actuation 3610 may be used to dislodge trapped bubbles in the connectors 3608.

For example, when the overall fluidic process of interest is to supply the cassette 3604 with fresh media from the left-hand volume 3606a and extract spent media into the right-hand volume 3606b, instead of simply pushing liquid from the left-hand side through the cassette 3604 into the right-hand side, which would cause trapped bubbles to enter into the fluidic chamber, the initial push may be made with a series of back-and-forth liquid motions that serve to dislodge bubbles 3612 in the connectors 3608 and float them up into a gas head 3614 of the volume 3606a. Additionally, tapping or vibration may be applied to the connector area (indicated by arrows 3616) to further dislodge gas bubbles. The entire plugging, de-bubbling, and liquid transfer operation may be performed by an automated system.

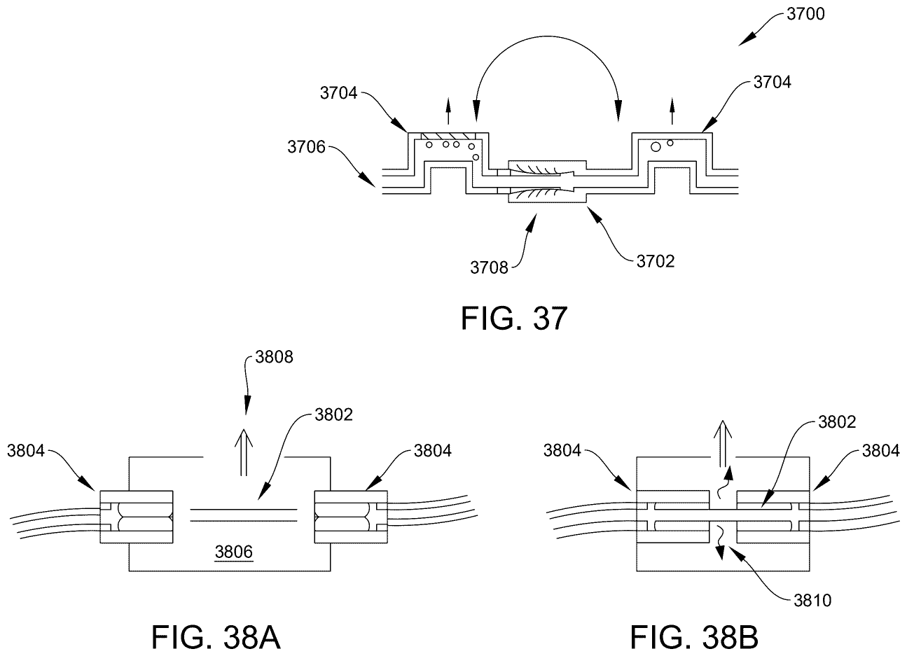

FIG. 37 is a diagram illustrating a bubble removal system 3700 in accordance with various implementations. The system 3700 is suitable for horizontal operation. The system 3700 includes a self-sealing pluggable connector 3702, where bubbles may be trapped during the connection process. There are bubble-trapping chambers 3704 on both sides of the connector 3702, although in some implementations they may only be present on one side (for example, on the liquid supply side and not on a fluidic cassette side). After connection, liquid volume may be translated via pressure/volume displacement towards a bubble trap, potentially with oscillation (indicated by arrow 3706) to dislodge any trapped bubbles in the connector. Additionally, tapping or vibration (indicated by arrow 3708) may be applied to the connector area to help dislodge bubbles. Additionally, the assembly may be oriented to apply additional gravitational forces to force bubble removal from the main liquid path. Bubbles trapped in the bubble traps 3704 may be subsequently removed by various means, including but not limited to the use of a gas-permeable membrane and an external vacuum or low-pressure environment to pull the gas content out of the liquid system.

FIGS. 38A-B are diagrams illustrating a system and method for removing bubbles in fluid connectors in accordance with various implementations. FIG. 38A shows an intermediate connector 3802 between two self-sealing pluggable ports 3804, within a sealed space 3806. After, for example, a sterilization cycle that sterilizes the sealed space 3806, a vacuum 3808 may be applied to the space 3806 prior to connection, to minimize the amount of gas trapped due to the plug-in connections. FIG. 38B shows the connector system after the plug connection has been made, with the intermediate connector 3802 fitting into and opening the self-scaling connectors 3804. The vacuum action 3808 may be further applied after formation of the connection, in conjunction with the intermediate connector 3802 that is gas-permeable such that any trapped bubbles in the connector 3802 are pulled out through the walls of the connector 3802, as indicated by arrows 3810. In some implementations, one or more sensors or cameras may be used to detect bubbles in the fluidic systems, which enables a closed-loop bubble detector. The bubbles may then be removed using any of the techniques disclosed herein.

Bioprocessing Systems with Pluggable Cassettes

The systems and methods disclosed herein include a series of systems for bioprocessing that are enabled by a pluggable, self-sealing (or normally-closed) fluidic cassette format. The cassettes include an automation-compatible mechanical carrier, internal fluidic portions, and fluidic connectors that are normally sealed to the external environment. The majority of the internal fluidic system may be filled with liquid such that orientation or motion of the cassette causes little internal liquid motion. There are a variety of advantages to the self-scaling pluggable cassette described herein. For example, such cassettes have the advantages of the fluidic chambers disclosed herein in terms of stability, control of conditions, superior image quality, and resistance to contamination, but also allow very flexible bioprocesses and equipment by enabling a variable number of chambers to be used in a process because fluidic handling may be done centrally. The cassettes minimize the amount of overhead fluid that is trapped in permanently connected tubing, and which must often be flushed from the system prior to use with cells. Furthermore, the cassettes enable equipment to be better shared between multiple fluidic chambers, and enable bioprocesses that use a series of fluidic chambers for a series of phases or operations, including chambers with different geometries, surface properties, or other functions. The cassettes also enable flexible and high-acceleration transport around equipment and systems, for example in systems with shared pieces of equipment. Lastly, the cassettes potentially enable a range of different functions on a cassette with a standardized pluggable interface.

Additionally, the present implementations include aseptic versions of the self-scaling pluggable cassettes as described herein, which have further advantages. For example, the aseptic cassettes may enable multiple batches, such as patient-specific batches, to be processed in the same environment (room, or system) without the possibility of cross-contamination. They also enable transport and handling of cassettes in non-sterile, low-grade cleanrooms, or even “controlled not classified” (CNC) facilities. This allows transport between different systems for the purpose of accomplishing different process phases, or for switchover in case of equipment downtime. Additionally, it allows transfers between automated systems and manual (isolated) steps where needed. The aseptic cassettes may also enable cassette-to-cassette coupling and/or materials transfers.

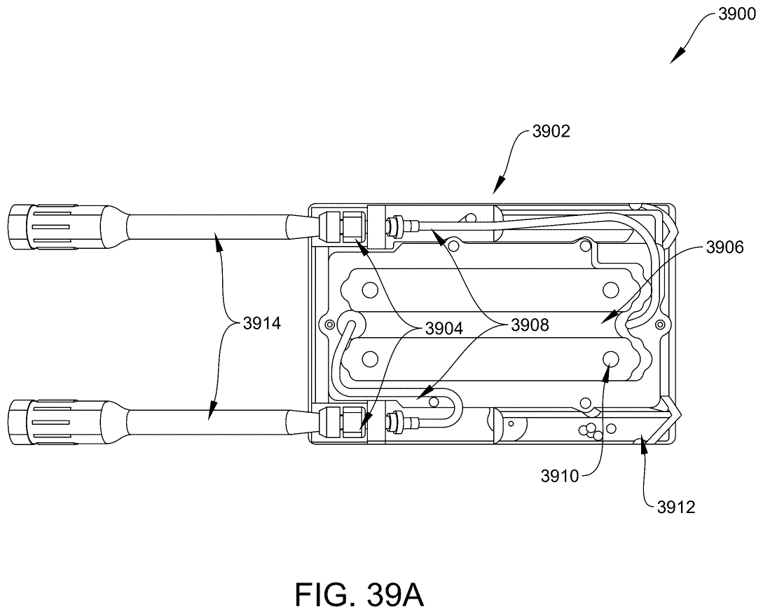

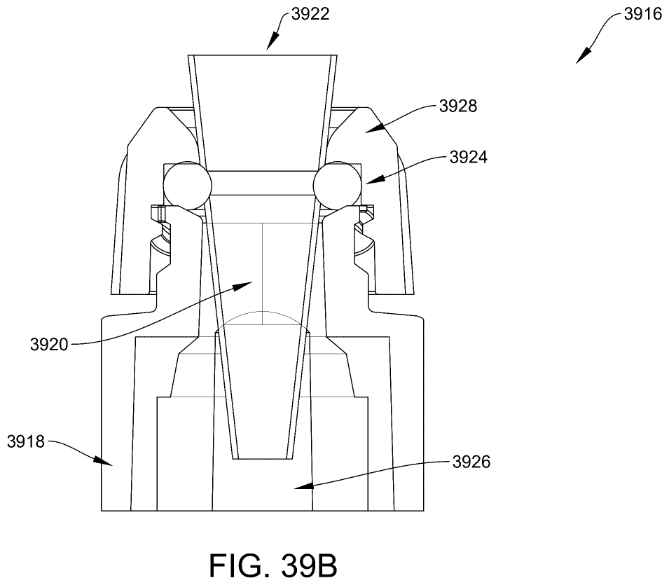

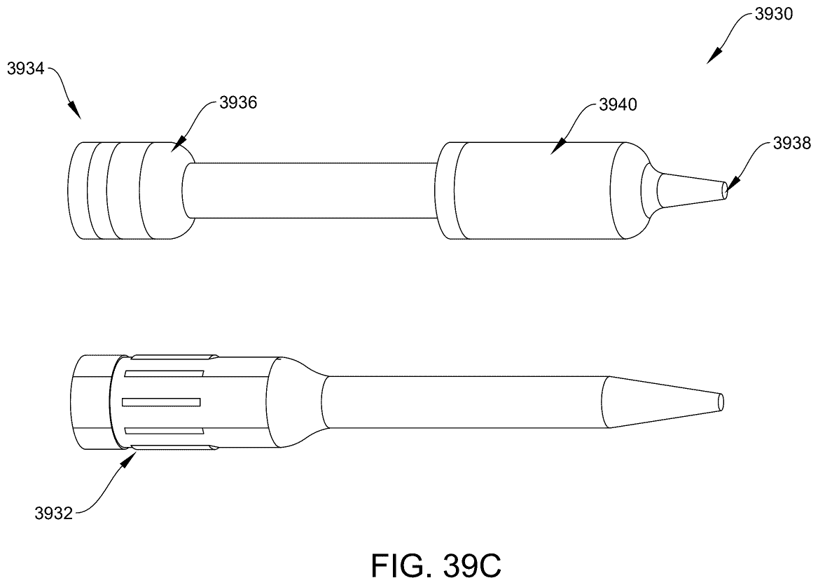

FIG. 39A is an image of an automation-compatible closed fluidic cell growth cassette system 3900 with normally-closed pluggable ports in accordance with various implementations. The system 3900 includes a cassette 3902 that has normally-closed pluggable ports 3904 configured to keep liquid contained onboard, and withstand a range of positive and negative pressures without leaking. In some implementations, the ports 3904 may utilize external actuation to open them for liquid or gas transfer. Such actuation may be provided by the transfer devices themselves (such as the implementation in FIG. 39A in which pipette tips are pushed into the ports to open them), or by a separate action (for example, an out-of-plane actuation that releases a pinched section of tubing just inside the port). In some implementations, a cap may further be applied to the connector to protect it from the environment and prevent cross-contamination, including various implementations that include aseptic pluggable connectors as described herein.

The cassette 3902 further includes a cell culture chamber 3906. In some implementations, the bottom surface of the chamber 3906 is a glass or fused silica window with a semitransparent nanometer-scale laser-absorbing film on the cell growth surface. This film is semitransparent for the purpose of imaging, but absorbs pulsed laser radiation to form microbubbles that porate cell membranes. In some cases, bubble energies or repetitions that cause irreversible poration may be used to kill selected cells. In the present implementation, the fluidic growth chamber may have dimensions of roughly 10 cm in length by 1 cm in breadth, by 1 mm in height, constituting approximately 10 cm² of adherent cell growth area, with approximately 1 mL of media volume. The top window of the cassette 3902 may be formed using a thin, optically-transparent polymer that further is sufficiently thin and gas-permeable (to oxygen, carbon dioxide, and potentially other gases) to allow gas exchange with the contained cell media. The chamber 3906 forms a volume that may be imaged and laser-scanned with high fidelity. Other implementations of the cassette 3902 with normally-closed pluggable ports may have a wide range of cell growth areas, for example smaller growth areas of ≤5 cm² for initial cell reprogramming, editing, or delivery of compounds, in which a small number of cells are used and reagents are expensive. In other implementations, much larger growth areas, for example growth areas of ≥25 cm² or ≥40 cm², may be used in the same mechanical footprint to expand a larger number of cells. In other mechanical footprints, chambers of ≥100 cm² may be configured for cell growth. In some implementations, multiple growth chambers may be configured on the same pluggable cassette, either as parallel growth chambers for the same cell population, or as independent growth chambers for parallel experiments or runs (with appropriate valving, etc. to keep the experiments separated). In some implementations, aseptic valves (reusable or single-use) may be integrated into the cassette to facilitate sampling a small fraction of media or cells during the cell culture process. Various implementations may use different heights, for example<1 mm height, including≤0.5 mm height or ≤0.25 mm height that confines media, cells, and reagents into a small volume for certain operations, and/or allow application of higher shear forces via fluidic flow. In other implementations, the fluidic channel heights are >1 mm, for example≥2 mm or ≥ 5 mm, to provide a larger volume of media to sustain cells, thereby reducing the number of media operations required per unit time.

The chamber 3906 also is attached to the pluggable ports 3904 via tubing 3908 which allows a range of operations to be performed in the cell culture chamber, including but not limited to pre-treatment for cleaning, pre-treatment with anti-adhesion or pro-adhesion chemistries, coating with extracellular matrix, cell seeding, delivery of factors or other components (such as viral vectors, lipid nanoparticles, etc.), exchange of cell media, cell washing for media exchange and/or debris removal, and cell harvest. Other features of the cassette 3902 may include mini-windows 3910 exposing fiducial marks that have been patterned into the glass substrate and/or laser film that are used to register images and/or provide image autofocus calibration as described previously. The cassette 3902 may additionally include a frame 3912 that is automation-compatible. For example, the frame 3912 may be compatible with transport robots, imaging and laser scanning equipment, liquid handling equipment, incubation equipment, and mechanical actuation equipment (for shaking, tapping, rotation, etc.). In the present implementation, the frame 3912 may be compatible with SLAS/ANSI/SBS standards for multiwell plates, which makes it compatible with a wide range of equipment. This aspect of the present implementations makes it compatible with a wide range of existing instruments for process development.

Another aspect that provides back-compatibility for process development is the combination of the normally-closed ports and standard disposable pipette tips, for example the Hamilton 5 mL pipette tips 3914 shown here. To enable use of pipette tips and in some cases existing automated pipetting modules, the cassette 3902 may be oriented into a vertical position prior to insertion of the pipette tips 3914. Once inserted, the pipettes 3914 open the pluggable ports 3904, but also provide well-sealed connections such that pressures and vacuum may be applied, and pressure differentials across the cassette 3902 may be used to transport liquid through the cassette 3902 and chamber 3906. This format of the chamber 3906 may be designed for backward-compatibility with existing equipment to speed up process development in fluidic cell growth and processing. The format may be compatible with four or more fluidic connections made with pipette tips, enabling a wide range of functionalities in a small footprint.

FIG. 39B is a diagram of a normally-closed port 3916 used in the cassette 3902 shown in FIG. 39A (e.g., ports 3904) in accordance with various implementations. In the present implementation, a Qosina™ part number 80213 (needleless injection site, swabbable, Luer lock) component is used, with some modification for sealing. An external polycarbonate port housing 3918 houses a silicone split-septum seal 3920 that may be pierced by an external channel, in this example an automation-compatible disposable pipette tip 3922. As the pipette tip 3922 is pushed into the port 3916, the split septum seal 3920 is opened, enabling fluid flows to/from the pipette 3922 into the cassette. An additional O-ring 3924 ensures proper sealing of the connection to allow positive/negative pressures necessary to provide liquid/gas flows or positive or negative pressures in the cassette, as described herein, through the chamber-connected volume 3926. The O-ring 3924 may be held in place with a screw-on retaining component 3928, which may also serve to position and/or mate with the receiving fluidic connector.

FIG. 39C is an image of an example implementation of automation-compatible pipette tips 3930 that are compatible with the cassette 3902 shown in FIG. 39A in accordance with various implementations. FIG. 39C shows an off-the-shelf Hamilton 5000u L conductive pipette tip (model #184020) 3932. FIG. 39C also shows a customized pipette tip 3934, compatible with the Hamilton ZEUS automated pipetting module (5 mL capacity). The pipette tip 3934 has a section 3936 that fits the pipetting module, and a front tip 3938 that is identical to the standard tip, so as to fit into the same normally-closed pluggable port. In this example, the tip 3938 has a section 3940 that is wider for the purpose of containing liquid with an air head in a wider-diameter volume to “de-bubble” the pluggable connection prior to liquid exchange, as described herein. Other configurations of pipette tips compatible with normally-closed pluggable cassettes are also contemplated herein.

Bioprocessing Systems with Pluggable Cassettes

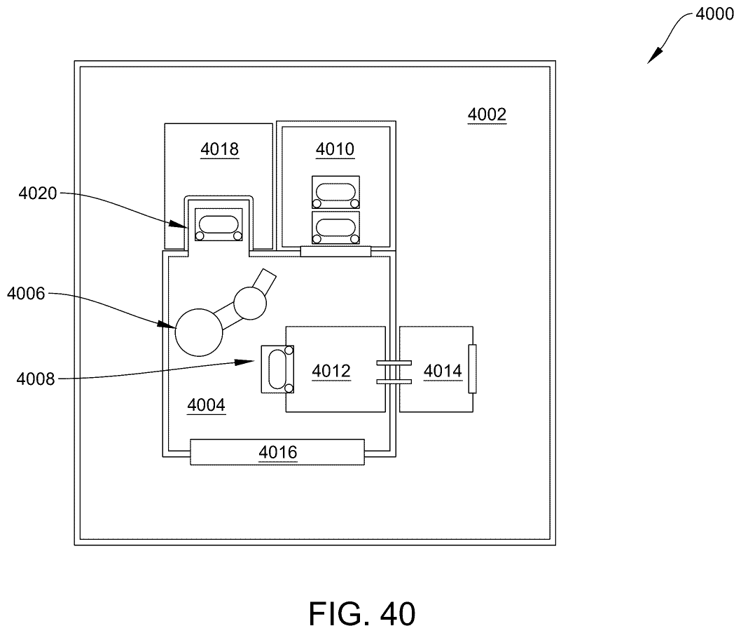

FIG. 40 is a diagram of an operating environment 4000 (e.g., system 100) enabled by pluggable fluidic cassettes in accordance with various implementations. The environment 4000 may be configured to support a single cell batch (for example, autologous patient batch) per environment. The present implementations detail the use of the environment 4000 in a clinical process according to current good manufacturing practices (cGMP), in which a cell batch is processed in a dedicated ISO Class 7 cleanroom 4002. Within this cleanroom 4002, there is a workcell 4004 with a clean environment with air handling to create an ISO Class 5 sub-environment (equivalent to a biological safety cabinet, or BSC) to serve as the core of the semi-automated system. Within the workcell 4004, an automated transport system 4006 is positioned to move cassettes around to different subsystems. In some implementations, the transport system 4006 may be a multi-axis robot for maximum flexibility. The transport system 4006 may, besides moving cassettes to different subsystems, also provide mechanical actuation functions such as “tapping” or “rotation” that have been described herein.

The workcell 4004 also includes a plurality of fluidic cassettes 4008, each cassette having a cell growth or processing chamber and at least two normally-closed pluggable ports (for example, as illustrated in FIG. 39A). The use of this consumable format allows robotic transport without sloshing of contents. In other words, the closed fluidic cassette format constrains media and cells between two surfaces and greatly reduces fluid flow, shear stress, and agitation during typical cassette manipulations, such as removal from an incubator by a fast-moving robotic arm. The cassette format also allows use of the operating environment 4000, including automated incubator 4010, without high humidity normally required when using open-top consumables. In this BSC-equivalent design, the incubator 4010 may maintain a temperature of 37° C. or other desirable cell culture temperature, as well as CO₂ and/or O₂ concentrations. The incubator 4010 may be maintained at low humidity to prevent growth of potential contaminating organisms. This is enabled by the sealed nature of the pluggable cassette, with most of its components largely impermeable to water vapor, while in some cases allowing gas exchange through a component such as a clear polymer window, as described herein.

The workcell 4004 also includes a liquid handling system 4012 that is configured to transact with the cassettes 4008 to change cell media, perform washings, seed or harvest cells, etc. Interfacing and liquid transactions with the cassettes 4008 may be performed with disposable pipette tips, as described herein. Connected to the liquid handling system 4012 is, optionally, a media storage and supply system 4014 configured to maintain media at 4° C., and warm and/or equilibrate media as described herein. This system 4014 allows access by the user to exchange liquids as indicated by a door on its right face. The system 4014 may also maintain a reservoir of phosphate-buffered saline (PBS) or other liquids for washing operations. Waste liquid may also be drained to a parallel storage container. A sash or door 4016 may be used to manually load or unload consumables and tubes with cells, small-volume liquids or reagents, with appropriate cleanliness precautions (when used in a clinical setting) such as would be used when using a BSC in a cGMP cleanroom.

The workcell 4004 also includes an optical engine 4018 configured to provide imaging functions, and in some implementations, laser scanning functions that selectively process (including kill) cells within the fluidic cassettes 4008. An aspect of the present implementation is that the workcell 4004 includes a clear box 4020 that protrudes into the optical engine 4018, that allows imaging and scanning using the optical engine 4018 that is external to the interior of the workcell 4004. This isolates the optical engine 4018 from biological materials. The present implementations allow the partial automation of a range of bioprocesses, in which imaging, scanning, washing, media changes, harvest, etc. may be performed autonomously or via remote supervision. This drastically reduces the amount of hands-on labor, which involves extensive gowning and decontamination routines. It also allows one skilled biologist to review images and cell maps from multiple such units remotely, rather than entering the cleanroom. The lower staffing in the cleanroom in turn increases cleanliness and reduces the chance of contamination. In addition, the use of closed fluidic cassettes 4008 with normally-closed pluggable ports drastically reduces the possibility of contamination, while also providing a more consistent operating environment, allowing maximum control of the process via imaging, mapping, software algorithms, expert viewing, and laser cell removal. It also enables precise, well-controlled fluidic transactions that have much more predictable shear forces, media and particle distributions, etc. than open-container liquid transactions.

For example, to accomplish a weekend of bioprocessing on eight cassettes carrying cell cultures, 32 disposable pipette tips may be placed into the enclosure prior to the weekend (four pipette tips per cassette). Remote monitoring may be performed via images from the optical engine 4018, sensor readings from various workcell components, and/or cameras installed in the workcell 4004. The workcell 4004 and the surrounding environment (cleanroom) may be sterilized and cleaned extensively between cell batches. The environment 4000 and its constituent systems may run using either non-aseptic or aseptic normally-closed fluidic connectors on the cassettes 4008. While the operating environment per cell batch is isolated, it may in some cases be advantageous to use pluggable, reusable aseptic connectors to further protect cassette contents from contamination, and to allow transfers of cassettes from environment to environment through uncontrolled or lower-grade cleanrooms.

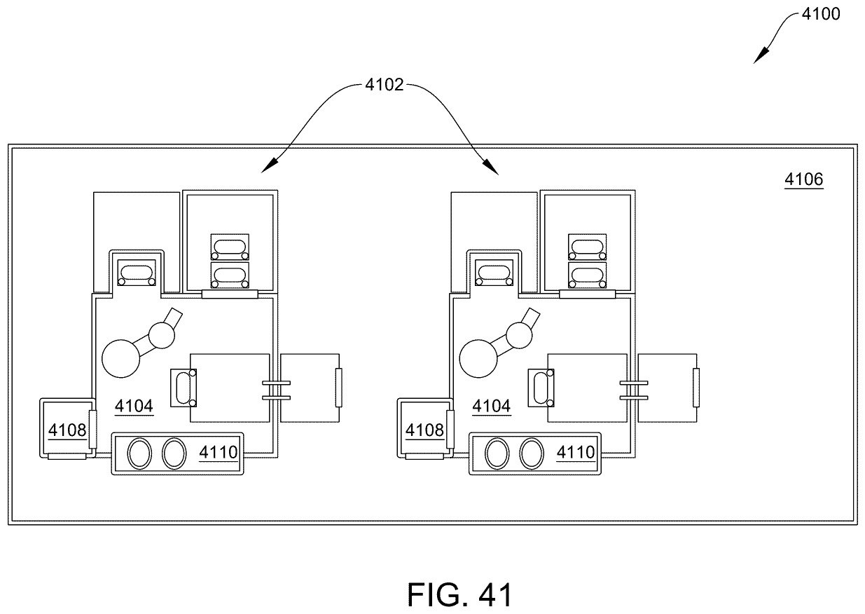

FIG. 41 is a diagram illustrating another operating environment 4100 for performing batched bioprocesses (e.g., autologous bioprocesses) in accordance with various implementations. The environment 4100 includes a plurality of semi-automated isolator workcells 4102 that operate using normally-closed fluidic cassettes (e.g., the cassettes described with reference to FIG. 39A). The workcells 4102 may be similar to the workcells described with reference to FIG. 40. In present implementations, the primary workspace where cassettes are handled and transported between workspaces 4104 is isolated from the surrounding room 4106, with access only through a load lock 4108, which includes features for sterilization. For example, each load lock 4108 may include a gas-based sterilization system. Supplies are bagged and placed into the load lock 4108, a gas cycle sterilizes the containing bag, and after gas flushing, the supplies may be transferred to the isolated workspaces 4104 and taken out of the bag, through use of the sterile glove ports 4110. The glove ports 4110 allow certain periodic operations (e.g., bagging, unbagging, opening/closing of vials, placement of consumables, etc.) to be performed manually to increase flexibility and keep the development cost of contained automation and robotics low. However, the majority of cell culture and processing operations may be run autonomously and/or under remote control.

An aspect of the present implementation is that because it operates as an isolator, the room 4106 that constitutes the operating environment may be a lower-grade cleanroom, such as an ISO Class 8 cleanroom, or an ISO Class 9 cleanroom, or even a CNC environment.

Importantly, multiple isolated workcells 4102 of this design may be operated in the same environment 4100, so that operators may service multiple workcells 4102 and therefore multiple cell batches with a single controlled entry into the environment 4100. This may also reduce the overall footprint of the cell processing facility. In some examples, workcells 4102 may be operating the same bioprocess (for example, patient iPSC reprogramming) in parallel. In other examples, the workcells 4102 may perform different portions of a process in a pipelined manner (for example, iPSC reprogramming, iPSC expansion, and iPSC differentiation into a target cell type). The internal portions of the workcell 4102 may be sterilized between patient batches. In some implementations, all workcells 4102 and the environment 4100 may be sterilized and decontaminated simultaneously.

In some implementations, the isolator workcells 4102 are designed to enable closed sterilization, allowing them to be sterilized while other workcells in the environment 4100 continue to run bioprocesses. Sterilization may be performed by manual wiping of surfaces with liquids, and/or by gas-based sterilization such as a vaporized hydrogen peroxide (VHP) cycle. In some implementations, some components of the automated systems inside the workcells 4102 may be replaced entirely with new components, or components that have been sterilized outside of the environment 4100. The environment 4100 and its constituent systems may run using either non-aseptic or aseptic normally-closed fluidic connectors on the cassettes. While the operating environment per cell batch is isolated, it may in some cases be advantageous to use pluggable, reusable aseptic connectors to further protect cassette contents from contamination, and to allow transfers of cassettes from environment to environment through uncontrolled or lower-grade cleanrooms.

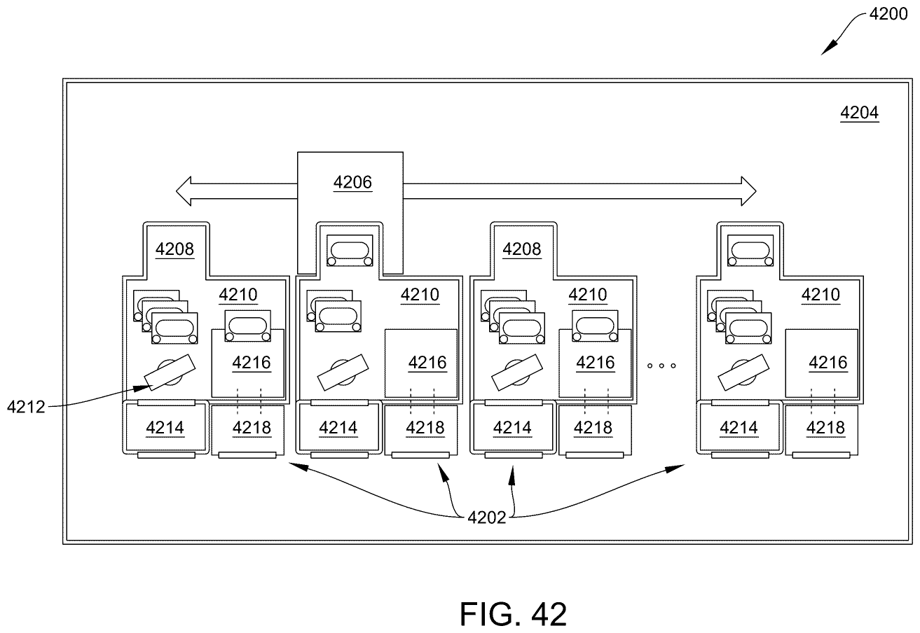

FIG. 42 is a diagram illustrating another operating environment 4200 for pluggable cassette-based systems in accordance with various implementations. The environment 4200 includes a plurality of single-batch processing workcells 4202 used together in a single room 4204, which may be a cleanroom, a low-grade cleanroom, or a CNC space. In the example shown in FIG. 42, a single optical engine 4206 is shared among multiple workcells 4202 to reduce overall costs. The optical engine 4206 may be automatically translated into position based on an overall schedule for each workcell cluster. Different configurations may have a different number of workcells 4202 per optical engine 4206, including configurations in which each workcell has a dedicated optical engine. The optical engine 4206 operates around an optical observation/treatment box 4208 which is part of the isolated environment of each workcell 4202. Multiple clusters or workcells sharing optical engines may share the same environment.

An isolated workspace 4210 of each workcell 4202 may be maintained at cell temperature and at appropriate gas concentrations, such that cassettes may be incubated in the central space rather than in a dedicated incubator. A transport subsystem 4212 in each workcell 4202 may be configured to move cassettes around the interior of the workspace 4210, and load/unload items from load locks 4214. Items may be pre-palletized such that automated loading, use, and unloading is possible. The transport subsystems 4212 may be integrated with a liquid management system 4216, such that a single set of actuators may be used for the entire lifecycle of a cassette in the workcells 4202.

The liquid management systems 4216 may be connected to liquid/reagent/waste storage subsystems 4218 for each workcell 4202. This may be done via tubing, or via a pallet-based setup and load lock with sterilization as described herein. The workcells 4202 in this implementation may include capping/decapping and other tube handling automation to automate transfers of small amounts of reagents, cells, or liquid samples for analysis of ongoing bioprocesses. The environment 4200 and its constituent systems may run using either non-aseptic or aseptic normally-closed fluidic connectors on the cassettes. While the operating environment per cell batch is isolated, it may in some cases be advantageous to use pluggable, reusable aseptic connectors to further protect cassette contents from contamination, and to allow transfers of cassettes from environment to environment through uncontrolled or lower-grade cleanrooms.

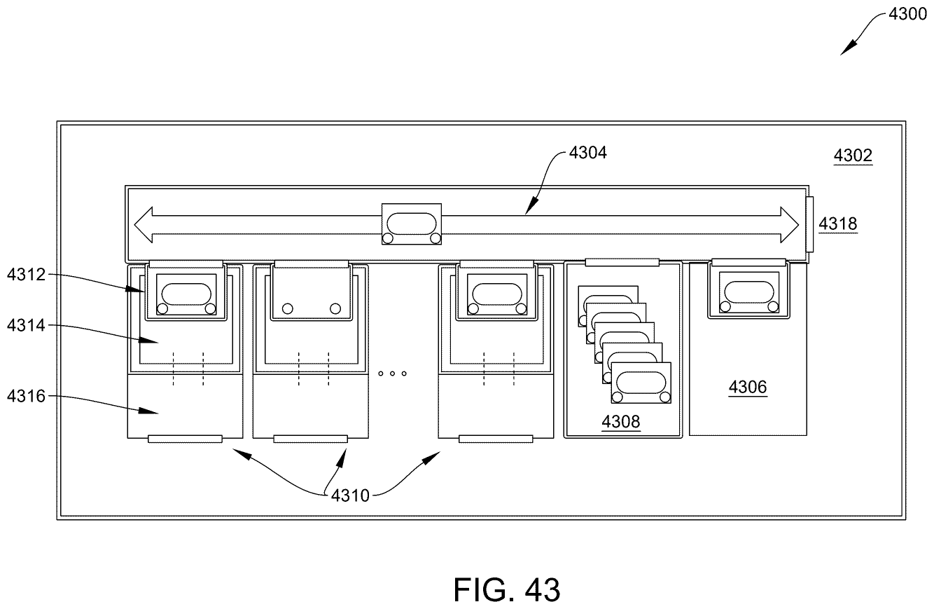

FIG. 43 is a diagram illustrating another operating environment 4300 utilizing shared equipment by multiple cell batches in accordance with various implementations. The implementation shown in FIG. 43 is dependent on reusable aseptic pluggable connectors on cassettes for the purpose of preventing cross-contamination between batches when using shared equipment. The operating environment 4300 includes a room 4302 that may be a low-grade cleanroom or CNC facility, and a transport plane 4304 configured to move cassettes, around which a series of process modules are arranged. Multiple systems configured around multiple backplanes may share the same environment. The modules are designed in a manner that allows a system to be flexibly configured, and modules may be exchanged at will, even during operation, both for maximizing uptime and reliability, and for bioprocess flexibility. Modules may include but not be limited to: one or more optical engines 4306 which provide imaging and/or laser scanning functionalities and/or other cell removal tool modules that enable cell removal within sealed fluidic cassettes; incubator modules 4308 that incubate multiple cassettes, including in some cases cassettes carrying different cell batches (for example, batches belonging to different patients in autologous bioprocesses); and fluid management modules 4310 which may be batch- or patient-specific fluid management modules.

Each fluid management module 4310 may include a cassette handling area 4312 which includes an aseptic connector system that attaches to the cassette as described herein and a fluidic handling system 4314 which manages fluidic and gas transactions with the cassette, and manages other functions such as mixing, washing, cell seeding, cell harvesting, etc. The fluidic handling system 4314 is attached to a fluidic storage subsystem 4316 which stores media, reagents, washing liquids, cell samples, etc. as described herein. The fluidic storage system 4316 is accessible externally to facilitate changes in liquids and loading/unloading of reagents and consumables, where applicable. This may be accomplished by bulk bags/containers attached via tubing, and/or individual containers that are presented to the fluid handler. Cassettes may be loaded or unloaded into each module via door 4318 at the end of the cassette transport plane 4304. The transport, incubation, and other portions of the system will generally be kept clean using filtered airflow, for example at ISO Class 7 or even ISO Class 5 levels, but this is only an extra precaution, because the aseptic nature of the cassettes allows them to be handled in CNC spaces. This includes transfers from one system to another, in the case that a bioprocess is accomplished on multiple systems, or in cases in which cassettes are switched over from one system to another for maintenance, cleaning, or repair of the system. The modular nature of the system, however, allows individual modules to be maintained in place, or detached and maintained, without interrupting the operation of the system.

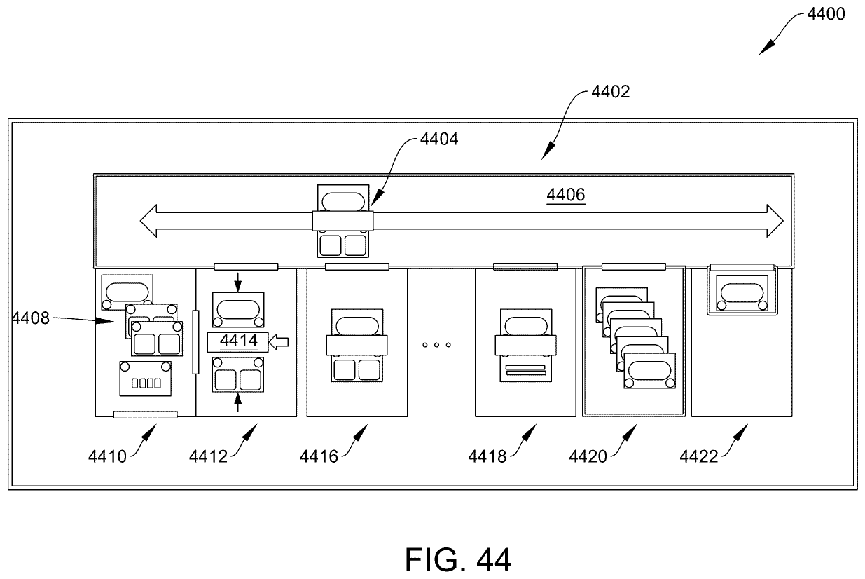

FIG. 44 is a diagram illustrating another operating environment 4400 supporting an aseptic normally-closed pluggable cassette-based system 4402 in accordance with various implementations. Multiple such systems may share the same environment. The system 4402 operates based on an aseptic coupling concept in which cassettes are coupled together and transported to various process modules in coupled configuration 4404. The coupled cassette configuration may include a fluidic cell culture cassette (top cassette) and a media/waste cassette (bottom cassette) attached to an intervening coupler. In this configuration, the coupled cassettes 4404 may be aseptically sealed to one another, and fluids, gases, cells, etc. may be exchanged for various operations. Cassettes, either individually or in aseptically-coupled form, are transported via backplane 4406 that connects multiple functional modules, to provide a highly-configurable and reliable system architecture.

Cassettes 4408 are loaded via an access port on a cassette management/storage system 4410, which may include multiple temperature zones, etc. Multiple types of cassettes may be loaded, as described herein, including cell growth cassettes and media and waste cassettes, which may be single-use (i.e., perform a single media change on a single growth cassette), multi-use cassette-dedicated (i.e., perform multiple media changes on a single cassette over a period of time), and/or multi-use batch-dedicated (i.e., perform multiple media changes on multiple cassettes containing the same cell batch). The cassettes 4408 may also include function-specific cassettes, which can provide a range of functions including but not limited to cell sorting, filtration, intracellular delivery, spheroid or droplet formation, etc.

One or more aseptic coupling modules 4412 communicates with the cassette storage system 4410 and transport backplane 4406, and provides aseptic coupling and uncoupling functions between pairs of cassettes. This may include the mechanical connection of the cassettes to a coupler 4414, sterilization routines (via gas, liquid, heat, plasma, UV, etc. as described herein), and sterile liquid connections between the two cassettes. In some implementations, 3+cassette coupling formats are contemplated, for example in which multiple cassettes in a single batch are connected to one media or cell source cassette. In some implementations, the coupling modules 4412 may include storage for cassettes that are in the process of being sterilized, in which the sterilization action requires residence of a chemical sterilizer (or in other implementations may be a separate module in the system 4402).

Once coupled with the sterile liquid/gas connections established, the ensemble may be transported to one or more functional modules 4416, which may, for example, represent a media exchange module, in which media is warmed and equilibrated and then pushed into the cell growth cassette, with the waste collected. Other modules 4418 may handle other operations, which may include different pumping, valve actuation, electrical or optical connections to specialized modules, imaging, laser systems, etc., to accomplish a wide range of operations. In this manner, a large range of complex bioprocesses may be accomplished using a common system architecture and aseptic connection system, by adding special-purpose cassettes and functional modules. The system shown in FIG. 44 enables many different bioprocesses to be supported by adjusting the configuration of modules and cassettes, and running different processing routines. Other modules, including incubators 4420 and optical engines 4422, as described herein, may also be connected to the system 4402. Specialized incubators that utilize coupled cassette pairs for continuous perfusion media exchange (and temperature control of the two cassettes) may be used. Similarly, the optical engine design may include accommodation for coupled cassettes, in which imaging observation and/or laser processing may be combined with fluidic operations such as washing.

### Multipart Cell Culture Container and Methods of Use

The systems and methods described herein include methods for continuous maintenance of proliferative cell colonies, including splitting a cell colony into sub-colonies. The sub-colonies may be utilized for a variety of purposes, including expanding the overall number of cells, creating test colonies for applying perturbation-based measurement techniques (as described herein), manipulating sub-colonies (e.g., to differentiate them into different cell types), and harvesting cells to measure cell characteristics or for downstream processing and use. However, the environmental conditions for performing these functions on the sub-colonies may be different than the ideal conditions for long-term healthy cell colony maintenance of the “main” cell colonies. For example, the ideal media conditions, media flow rates, extracellular matrix conditions, surface roughness or patterning, etc., may be substantially different for long-term maintenance versus the cell colony operations disclosed herein. This creates a challenge in which performing non-maintenance activities may have a negative impact on the health, state, or even count of the maintained cell colonies.

To counteract this negative effect, the systems and methods disclosed herein include various cell culture chamber designs, associated fluidics, and appropriate cell process operations that allow conditions to be independently tailored for cell maintenance and other cell operations, including harvest of cells. The cell culture chamber designs may include compartments, chambers, or channels that are separated by partial barriers that allow maintenance of separate cell culture conditions, but allow transit of adherent cells from one compartment to another by proliferative growth. This transfer and growth may be controlled by a cell culture system that includes cell imaging subsystems, computing subsystems, and cell removal tools.

The systems and methods disclosed for independent environmental control of various cell culture regions have a variety of applications in cell culture systems. For example, these designs and techniques may be applied to colony measurement via test colony perturbation and/or harvesting of intact cell materials, while maintaining ideal growth conditions for the main cell colonies. In this implementation, cells that serve as test colonies for the main cell population are “transported” via selective removal and subsequent regrowth by the cell removal tool to a test region that is fluidically separated from the growth region. This allows for the application of various perturbations (as disclosed herein) to be performed on the test cells to deduce the state, quality, functionality, viability, etc., of the main cell culture, all without perturbing the main cell culture. In some implementations, this includes selectively harvesting cell material from portions of the main cell culture by applying cell harvesting techniques only to the test sub-colonies, so as not to perturb the main cell culture.

Another application for the designs and techniques disclosed herein is for continuous bioproduction, in which a proliferative population of cells is maintained in one set of cell culture chambers under one set of conditions ideal for healthy proliferation, and fractions of the cell population are continuously transported (with healthy density conditions) via selective cell removal tool to a second set of cell culture chambers. Various operations may be performed on the cell colonies in the second set of cell culture chambers. For example, cell materials may be harvested directly from the second set of cell culture chambers via a range of harvesting tools, including but not limited to mechanical or enzymatic means, but also potentially through destructive or non-destructive use of a cell removal tool. The product of the cell culture system that is harvested may be materials (e.g., proteins, viral vectors, DNA, etc.) that are produced by the cells or the live cells themselves. In another example, cells may be continuously differentiated and harvested in the second set of cell culture chambers, and the conditions in the second set of cell culture chambers may be optimized for such differentiation/modification/maturation. In an example implementation, iPS or embryonic stem cells may be proliferated in the first set of cell culture chambers and differentiated in the second set of cell culture chambers prior to harvest. In another example implementation, progenitor cells may be proliferated in a first set of cell culture chambers and moved to the second set of cell culture chambers for maturation and harvest.

Another application for the designs and techniques disclosed herein is for biosensing, in which cell material is continuously proliferated and managed in optimal conditions for continuous proliferation in a first cell culture chamber, and then moved using the techniques described here to a second cell culture chamber where they function as a biosensor. In such implementations, external agents are flowed into the second cell culture chamber where they interact with the “biosensor” cells while not interacting with the proliferating reservoir of cells in the first cell culture chamber. Biosensing may include cell health or proliferation monitoring, for example, or the use of reporters (such as fluorescent reporters) that have been engineered into the cell population for the purpose of detecting chemical or biological agents. The cells in this second biosensing chamber may then be periodically removed via the cell removal tool and fluid flow to “reset” the biosensing capacity with fresh cells from the proliferation (i.e., first) cell culture chamber. This process may be performed continuously in different sections of the two chambers, providing a continuous growth, transfer, biosensing, and removal loop.

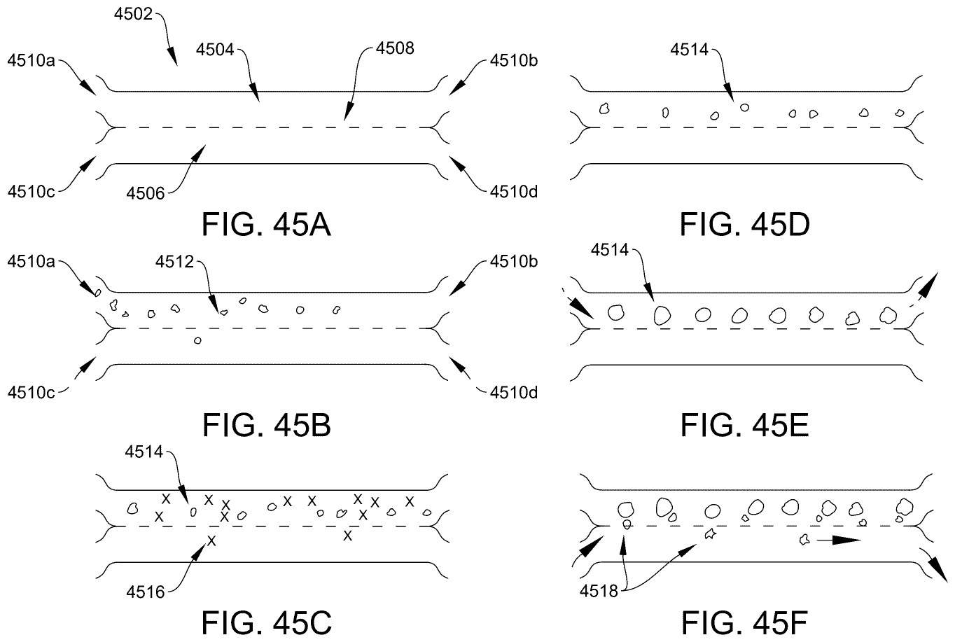

FIGS. 45A-F are diagrams illustrating utilization of a multi-part fluidic cell culture chamber in accordance with various implementations. FIG. 45A illustrates a fluidic cell culture chamber 4502 that is divided into two parts: a first sub-chamber 4504 and a second sub-chamber 4506 separated by a divider 4508. The divider 4508 may have multiple gaps or perforations large enough for growth of adherent cell colonies to traverse between the sub-chambers 4504, 4506. The cell culture chamber 4502 includes four fluidic ports 4510a-d that allow flows to be independently controlled in the sub-chambers 4504, 4506. The fluidic ports 4510a-d allow at least semi-independent control of media conditions in the sub-chambers 4504, 4506. For example, the fluidic ports 4510a-b may control media conditions in the sub-chamber 4504 while the fluidic ports 4510c-d may control media conditions in the sub-chamber 4506.

In some implementations, the divider 4508 may span most of the vertical height of the cell culture chamber 4502 (for example, a 0.75 mm total height), with a small gap along the cell culture surface (for example, a 0.05 mm height) that allows cell growth underneath the barrier and therefore allows proliferation from one sub-chamber to another. The divider 4508 may have multiple perforations, or in other cases may have a continuous low-height gap, that serves to separate flows in the sub-chambers 4504, 4506 and minimize cross-diffusion of media components while allowing cell growth to cross from one sub-chamber to another. Other implementations of the divider 4508 are also contemplated herein as long as it allows controlled growth of cells between the sub-chambers but substantially maintains separation of fluidic components and flows.

FIG. 45B illustrates seeding of cells or cell clumps 4512 primarily into the first sub-chamber 4504 using a fluidic flow created by the fluidic ports 4510a-b. During the flow-in process, the fluidic ports 4510c-d may maintain a closed position, or be used to apply a small amount of positive pressure within the second sub-chamber 4506 relative to the first sub-chamber 4504 to minimize escape of cells from the first sub-chamber 4504 to the second sub-chamber 4506.

FIG. 45C illustrates cell colonies 4514 that have attached to the growth surface of the cell culture chamber 4502 and started proliferating, as well as locations 4516 marked by an “X” at which certain cell colonies have been removed from the growth surface using the cell removal tool as disclosed herein. The removed cell colonies may include cell colonies that have emerged in the second sub-chamber 4506, as well as selected cell colonies in the first sub-chamber 4504. The cell removal may be performed, for example, by imaging the cell culture chamber 4502, generating a map of cell colonies (including position and potentially other features), and then using a cell removal tool to remove selected cell colonies, with debris washed out through the fluidic ports 4510a-d. The removal of cell colonies in the first sub-chamber 4504 may be guided by several objectives, including but not limited to achieving proper spacing between colonies, controlling distance to the barrier between sub-chambers, and selecting for desirable colony features.

FIG. 45D illustrates the cell colonies 4514 remaining after removal of some colonies by the cell removal tool. In some implementations, one or more colonies that remain may be positioned in the vicinity of the divider 4508. FIG. 45E illustrates proliferation of the cell colonies 4514 as the cells divide, with fresh media delivered into the first sub-chamber 4504. As described herein with reference to various cell operations that may be performed on the cell colonies 4514, the size, density, composition, and position of each cell colony 4514 may be controlled over time by repeated use of a cell removal tool. Over time, the cell colonies 4514 may be translated along the divider 4508, or kept in place through a series of cell removal operations that locally maintain each cell colony.

FIG. 45F illustrates the creation of sub-colonies 4518 from the cell colonies 4514 and their translation, via iterative use of a cell removal tool, from the first sub-chamber 4504 to the second sub-chamber 4506. In the example shown in FIG. 45F, the cell removal tool may be used to separate one or more sub-colonies 4518 from each cell colony 4514. The sub-colonies 4518 may be transported past the divider 4508 into the second sub-chamber 4506. In the second sub-chamber 4506, the sub-colonies 4518 may be subjected to a different set of conditions from those in the first sub-chamber 4504. The conditions that may be different include but are not limited to media components and state, media flow rates, surface properties including different extracellular matrix conditions, and different treatments (e.g., colony size and shape control) by a cell removal tool.

In some implementations, the conditions in the second sub-chamber 4506 may foster differentiation of the cells into a different cell type. For example, the first sub-chamber 4504 may contain rapidly proliferating pluripotent cells, and the second sub-chamber 4506 may serve as an area for continuous production of differentiated cells from the pluripotent cells. In other implementations, the second sub-chamber 4506 may enable continuous harvesting of cells from the main cell colonies 4514 while maintaining the main cell colonies 4514 in ideal proliferation and/or maintenance conditions. In such a case, differing surface conditions, use of enzymatic agents in the media, and higher flow rates in the second sub-chamber 4506 may be used to efficiently harvest sub-colonies 4518 that have traversed the divider 4508. In other implementations, the second sub-chamber 4506 may be used to expose the sub-colonies 4518 to perturbations (relative to the conditions in the first sub-chamber 4504) as disclosed herein to track changes in sub-colony features or dynamics and to deduce the state or quality of the corresponding main cell colonies 4514.

In other implementations, cell material from the sub-colonies 4518 may be harvested through a fluid flow in the second sub-chamber 4506. Sub-colonies 4518 may be simultaneously harvested, or processed and harvested one at a time to maintain separation, for example when using downstream cell assays to independently measure colony characteristics. In some implementations, once the quality of the main cell colonies 4514 has been determined through analysis of its associated sub-colony 4518, a selected main cell colony 4514 may be transported to the second sub-chamber 4506 and harvested, thus maintaining maximum isolation from other cell materials. In some implementations, a differential pressure between the first sub-chamber 4504 and the second sub-chamber 4506 may be applied to create high-velocity local flows across the divider 4508 that wash cells near the divider 4508 off the growth surface. These cells may be either immediately harvested through the fluidic port 4510d in the second sub-chamber 4506, or settle across the area of the second sub-chamber 4506 for additional proliferation and other cell processes.

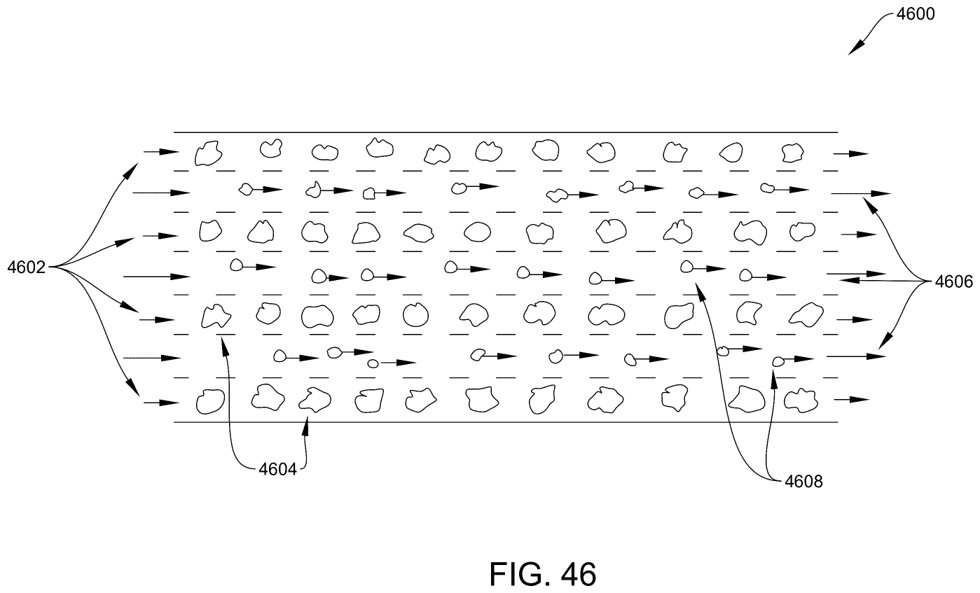

FIG. 46 is a diagram of another multi-part fluidic cell culture chamber 4600 in accordance with various implementations. The cell culture chamber 4600 (e.g., a fluidic closed cassette) may include multiple channels separated by partial barriers (similar to the divider 4508 in FIGS. 45A-F) that allow adherent cells to traverse them. For example, a first set of channels 4602 may be used to sustain proliferating colonies of cells 4604. The cells 4604 may be managed by a cell culture system using a cell removal tool that continuously splits off sub-colonies 4608 from the main colonies and translates them across the partial barriers into a second set of channels 4606.

The second set of channels 4606 may be used to harvest the sub-colonies 4608 that have been transferred into them. The harvest may be accomplished by use of a different (or different level of) extracellular matrix, by high flow rates, by use of the cell removal tool, by enzymatic agents, or by combinations of these. The harvested sub-colonies 4608 may then be flowed out of the cell culture chamber using one or more fluidic ports. The design of the cell culture chamber 4600 may allow proliferative cells to be seeded and continuously maintained in ideal growth conditions in the first set of channels 4602, potentially monitored and down-selected by the cell removal tool if drift from ideal characteristics is identified, and material from the cell colonies 4604 to be continuously harvested using the second set of channels 4606 with conditions favorable to cell harvesting rather than cell growth.

In some implementations, the second set of channels 4606 may additionally be used to differentiate or otherwise modify the sub-colonies 4608 prior to harvest. In some implementations, there may be more than two sets of channels, each dedicated for particular processes (e.g., processes that have cell maintenance, cell processing, and cell harvesting stages occurring in different conditions and therefore within different channels). In some implementations, the maintained cell colonies 4604 are adherent, but conditions in the non-maintenance channels (e.g., channels 4606) may be configured to produce non-adherent cells or cell clusters, including but not limited to spheroids or embryoid bodies. As disclosed herein, cell removal tools may be used in conjunction with other conditions to convert cells to non-adherent forms. For example, a differentiation process may convert cells from adherent to non-adherent form.

### Other Considerations

While various implementations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the implementations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings are used. Those skilled in the art will recognize, or be able to ascertain using no more than routine experimentation, many equivalents to the specific inventive implementations described herein. It is, therefore, to be understood that the foregoing implementations are presented by way of example only and that, within the scope of the appended claims and equivalents thereto, implementations may be practiced otherwise than as specifically described and claimed. In addition, any combination of two or more such features, systems, aspects, articles, materials, kits, and/or methods, if such features, systems, aspects, articles, materials, kits, and/or methods are not mutually inconsistent, is included within the inventive scope of the present disclosure. Particularly, any element of the disclosure and any aspect thereof may be combined, in any order and any combination, with any other element of the disclosure and any aspect thereof.

The above-described implementations can be implemented in any of numerous ways. For example, the implementations may be implemented using hardware, software or a combination thereof. When implemented in software, the software code can be executed on any suitable processor or collection of processors, whether provided in a single computer or distributed among multiple computers.

As used in any implementation herein, a “circuit” or “circuitry” may include, for example, singly or in any combination, hardwired circuitry, programmable circuitry, state machine circuitry, and/or firmware that stores instructions executed by programmable circuitry. An “integrated circuit” may be a digital, analog or mixed-signal semiconductor device and/or microelectronic device, such as, for example, but not limited to, a semiconductor integrated circuit chip.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smartphone or any other suitable portable or fixed electronic device. Also, a computer may have one or more input and output devices. These devices can be used, among other things, to present a user interface. Such computers may be interconnected by one or more networks in any suitable form, including a local area network or a wide area network, such as an enterprise network, an intelligent network (IN) or the Internet. Such networks may be based on any suitable technology and may operate according to any suitable protocol and may include wireless networks, wired networks or fiber optic networks.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

Implementations of the methods described herein may be implemented using a processor and/or other programmable device. To that end, the methods described herein may be implemented on a tangible, non-transitory computer-readable medium having instructions stored thereon that when executed by one or more processors perform the methods. The computer-readable medium may include any type of tangible medium, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of media suitable for storing electronic instructions.

The terms “program” or “software” are used herein in a generic sense to refer to any type of computer code or set of computer-executable instructions that can be employed to program a computer or other processor to implement various aspects of implementations as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various implementations. Also, data structures may be stored in computer-readable media in any suitable form.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. As used herein in the specification and in the claims, “or” should be understood to have the same meaning as “and/or” as defined above.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “coupled” as used herein refers to any connection, coupling, link or the like by which signals carried by one system element are imparted to the “coupled” element. Such “coupled” devices, or signals and devices, are not necessarily directly connected to one another and may be separated by intermediate components or devices that may manipulate or modify such signals. Likewise, the terms “connected” or “coupled” as used herein in regard to mechanical or physical connections or couplings is a relative term and does not require a direct physical connection.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems.

It will be appreciated by those skilled in the art that any block diagrams herein represent conceptual views of illustrative circuitry embodying the principles of the disclosure. Similarly, it will be appreciated that any flow charts, flow diagrams, state transition diagrams, pseudocode, and the like represent various processes which may be substantially represented in computer-readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

We claim:

1. A fluidic exchange system for cell culturing, comprising:
a cell culture cassette, comprising:
a cell culture chamber having a cell culture and fluid media therein; and
one or more pluggable ports fluidically connected to the cell culture chamber;
a fluid media cassette, comprising:
one or more liquid storage compartments; and
one or more pluggable ports fluidically connected to the one or more liquid storage compartments; and
a cassette coupler configured to fluidically couple the cell culture cassette and the fluid media cassette.

2. The system of claim 1, wherein the cassette coupler aseptically connects with the one or more pluggable ports of the cell culture cassette and aseptically connects with the one or more pluggable ports of the fluid media cassette.

3. The system of claim 1, wherein a first liquid storage compartment of the fluid media cassette stores fresh fluid media to replenish the fluid media in the cell culture chamber of the cell culture cassette.

4. The system of claim 1, wherein a second liquid storage compartment of the fluid media cassette receives used fluid media from the cell culture cassette.

5. The system of claim 1, further comprising a liquid transfer handler configured to initiate fluid flows between the cell culture cassette and the fluid media cassette.

6. The system of claim 5, wherein the liquid transfer handler is configured to circulate media in at least one of continuous flow mode and stopped-flow mode.

7. The system of claim 1, wherein the fluid media cassette further comprises at least one bypass valve between a first liquid storage compartment and a second liquid storage compartment to enable fluid mixing operations.

8. The system of claim 1, wherein the fluid media cassette further comprises at least one pinch valve between a first liquid storage compartment and a first pluggable port.

9. The system of claim 1, wherein the fluid media cassette further comprises a semiporous membrane configured to filter flows between a first liquid storage compartment and a second liquid storage compartment.

10. The system of claim 1, wherein the cell culture is adhered to a semi-transparent surface of the cell culture chamber.

11. The system of claim 10, wherein the semi-transparent surface comprises an optical film for optical imaging of the cell culture and optical cell culture management.

12. The system of claim 1, further comprising a robotic system configured to move at least one of the cell culture cassette and the fluid media cassette to the cassette coupler.

13. The system of claim 1, wherein the cell culture cassette provides a closed, sterile cell culture environment.

14. The system of claim 1, wherein the fluid exchange system is configured to simultaneously replenish the fluid media in the cell culture cassette with fresh fluid media in the fluid media cassette and removes waste media from the cell culture cassette to the fluid media cassette.

15. The system of claim 1, wherein the one or more pluggable ports of the cell culture cassette and the one or more pluggable ports of the fluid media cassette are self-sealing.

16. The system of claim 15, wherein the cell culture cassette further comprises a gas-permeable membrane allowing gas exchange while maintaining sterility of the cell culture chamber.

17. The system of claim 1, wherein the cassette coupler comprises a sterilant port configured to introduce a sterilant therein.

18. The system of claim 5, wherein the liquid transfer handler comprises a peristaltic pump controlling fluid flow between the cell culture cassette and the fluid media cassette.

19. The system of claim 1, wherein the cell culture chamber comprises a temperature control material configured to maintain the cell culture cassette at a predetermined temperature during fluid exchange operations.

20. The system of claim 1, wherein the cell culture cassette comprises optical fiducial markers for automated alignment and imaging of the cell culture chamber.

21. A method of fluid exchange, comprising:
providing a cell culture cassette, the cell culture cassette comprising:
a cell culture chamber having a cell culture and fluid media therein; and
one or more pluggable ports fluidically connected to the cell culture chamber;
providing a fluid media cassette, the fluid media cassette comprising:
one or more liquid storage compartments; and
one or more pluggable ports fluidically connected to the one or more liquid storage compartments; and
fluidically coupling the cell culture cassette and the fluid media cassette via a cassette coupler.
